(19) United States
(12) Patent Application Publication
(10) Pub. No.: US 2020/0123560 A1
(43) Pub. Date: Apr. 23, 2020

(54) TRANSCRIPTIONAL STIMULATION OF AUTOPHAGY IMPROVES PLANT FITNESS

(71) Applicant: SweTree Technologies AB, Umeå (SE)

(72) Inventors: Peter BOZHKOV, Uppsala (SE); Elena MININA, Uppsala (SE); Panagiotis MOSCHOU, Uppsala (SE); Daniel HOFIUS, Uppsala (SE); Sten STYMNE, Landskrona (SE)

(73) Assignee: SweTree Technologies AB, Umeå (SE)

(21) Appl. No.: 15/781,046

(22) PCT Filed: Dec. 2, 2016
(86) PCT No.: PCT/SE2016/051209

§ 371 Date: Jun. 1, 2018

(30) Foreign Application Priority Data
Dec. 4, 2015 (SE) 1551593-5

(51) Int. Cl. C12N 15/82; C07K 14/415

(52) U.S. Cl. CPC C12N 15/8273; C12N 15/8247; C12N 2310/20; C12N 15/8282; C07K 14/415; C12N 15/825

(57) ABSTRACT

The present invention provides to a method for enhancing the productivity of a plant by genetically modifying the genome of the plant to over-express at least one autophagy-related (ATG) protein selected from the group consisting of ATG5 and ATG7. The invention further provides a genetically modified plant characterized by over-expression of least one autophagy related (ATG) protein selected from the group consisting of ATG5 and ATG7. Additionally the use of a transgene encoding at least one autophagy related (ATG) protein selected from the group consisting of ATG5 and ATG7 for enhancing the productivity of a plant is disclosed.

### CROSS REFERENCE TO RELATED APPLICATIONS

This application is a U.S. National Phase patent application of PCT/SE2016/051209, filed Dec. 2, 2016, which claims priority to Sweden Patent Application No. 1551593-5, filed Dec. 4, 2015, the disclosures of which are incorporated herein by reference in their entirety.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 616562023900SEQLIST.TXT, date recorded: May 11, 2018, size: 304 KB).

### TECHNICAL FIELD OF THE INVENTION

The present invention relates to a method for enhancing the productivity of a plant by genetically modifying its genome to over-express at least one AuTophaGy-related (ATG) protein selected from the group consisting of ATG5 and ATG7. The invention further relates to a genetically modified plant characterized by over-expression of least one autophagy-related (ATG) protein selected from the group consisting of ATG5 and ATG7.

### BACKGROUND OF THE INVENTION

The productivity of a plant is the sum of several important traits that determine the rate of generation of biomass by the plant in the ecosystem it is cultivated. Key traits that contribute to enhanced plant productivity include: increased biomass production, delayed aging, enhanced vegetative growth, enhanced seed production, increased accumulation of storage lipids (including seed storage lipids); enhanced pathogen resistance and enhanced oxidative stress resistance.

Plant growth at apical meristems results in the development of sets of primary tissues and in the lengthening of the stem and roots. In addition to this primary growth, trees undergo secondary growth and produce secondary tissue “wood” from the cambium. This secondary growth increases the girth of stems and roots and contributes to the increased biomass production in trees.

Increasing the oil content in a crop plant is of great interest, due to the increasing consumption of vegetable oils for nutrition or industrial applications. Lipids and triglycerides are synthesized from fatty acids. Accordingly, there exists a need for (oil seed) crop plants producing seeds having a higher content of fatty acids; and wherein the total yield of seed derived fatty acids is increased.

The use of genetic modification to either introduce mutations in the genome of a plant or to introduce transgenes, has been used to generate plants with modified genotypes, from which to select plants with improved agronomic traits. However, the selected genetically modified plants obtained by the introduction of mutations in the genome of a plant or transformation with transgenes are limited to an improvement in only one or very few of the agronomic traits that contribute to enhanced plant productivity. For example transgenic approaches are known for increasing yield by specifically enhancing microbial disease resistance (Salmeron and Vernooij, 1998).

The maintenance of pure lines of genetically modified genotypes of selected plants comprising several mutations or introduced transgenes is an additional burden on the agricultural industry. Hence there exists a need for the identification of single genetic modifications, or transgenes that can be introduced into the genome of a plant, that confer on the genetically modified plant a wide range of advantageous agronomic traits that can, individually or in combination, result in an improved productivity of the plant.

Homeostasis of all biological systems, including plants, involves the turnover of the cellular components such that old or damaged macromolecules and organelles are replaced by the new ones. Most subcellular degradation is carried out by two mechanisms: the proteasome and autophagy. Autophagy has a capacity to degrade any proteins and protein complexes, as well as entire organelles, by sequestering a cargo in the double membrane vesicles, autophagosomes, and digesting the cargo upon fusion of autophagosomes with lysosomes or lytic vacuoles. The dynamic process of autophagosome formation, delivery of autophagic cargo to the lysosome or vacuole, and degradation defines an autophagic flux which can be measured experimentally by a number of specific assays (Klionsky et al. 2012).

The primary role of autophagy is to protect cells under stress conditions, such as starvation. During periods of starvation, autophagy degrades cytoplasmic materials to produce amino acids and fatty acids that can be used to synthesize new proteins or are oxidized by mitochondria to produce ATP, respectively, for cell survival. Under favorable conditions, low level of autophagic flux serves housekeeping function by clearing obsolete cytoplasmic contents. A repertoire of genes that control autophagy (termed ATG genes) was first discovered in budding yeast and later shown to be conserved in all eukaryotes including plants. The autophagy process costs energy. When autophagy is excessively induced, it can result in autophagic cell death, so-called type II programmed cell death (PCD). One fundamental conclusion that can be drawn from research on autophagy is that this process must be highly conserved and tightly regulated in natural conditions—too little or too much autophagy can be deleterious. Accordingly, although autophagy operates at the whole plant level to control re-cycling of cellular components for the reuse or energy production, the consequences of modifying its regulation are unpredictable and likely deleterious.

### SUMMARY OF THE INVENTION

The invention provides a genetically modified plant characterized by enhanced expression of one or more gene encoding an autophagy related ATG5 and/or ATG7 protein as compared to a corresponding wild type plant of the same species; wherein
a. the amino acid sequence of the ATG 5 protein has at least 80% amino acid sequence identity to a sequence selected from the group consisting of: SEQ ID No.: 2, 4, 6, 8, 10, 12, 14, 16, 18 and 20; and
b. the amino acid sequence of the ATG 7 protein has at least 80% amino acid sequence identity to a sequence selected from the group consisting of: SEQ ID No.: 22, 24, 26, 28, 30, 32, 34, 36, 38 and 40; and
   wherein each of the one or more gene comprises a promoter operatively linked to a coding sequence encoding the protein.

According to one embodiment the one or more gene encoding an autophagy related ATG5 and/or ATG7 protein is a native endogenous gene.

According to second embodiment the one or more gene encoding an autophagy related ATG5 and/or ATG7 protein is a transgene.

The genetically modified plant provided by the invention is characterized by one or more phenotypic features selected from the group consisting of: delayed senescence; increased vegetative growth; increased biomass production; increased seed production; increased seed lipid content; increased pathogen resistance; increased oxidative stress resistance; wherein the one or more phenotypic features are as compared with a corresponding wild type plant of the same species.

According to a preferred first or second embodiment the genetically modified plant is a crop plant or a woody plant.

The invention further provides a method for enhancing the productivity of a plant by genetic modification, comprising the steps of:

transforming the plant with at least one transgene encoding an autophagy related ATG5 and/or ATG7 protein; wherein
the amino acid sequence of the ATG 5 protein has at least 80% amino acid sequence identity to a sequence selected from the group: SEQ ID No.: 2, 4, 6, 8, 10, 12, 14, 16, 18, and 20; and
the amino acid sequence of the ATG 7 protein has at least 80% amino acid sequence identity to a sequence selected from the group: SEQ ID No.: 22, 24, 26, 28, 30, 32, 34, 36, 38 and 40;
wherein the at least one transgene comprises a promoter operatively linked to a coding sequence encoding the protein,
OR

introducing a promoter DNA molecule for operable linkage to one or more native endogenous genes encoding an autophagy related ATG5 and/or ATG7 protein in the genome of the plant; wherein
the amino acid sequence of the ATG 5 has at least 80% amino acid sequence identity to a sequence selected from the group: SEQ ID No.: 2, 4, 6, 8, 10, 12, 14, 16, 18, and 20; and
the amino acid sequence of the ATG 7 has at least 80% amino acid sequence identity to a sequence selected from the group: SEQ ID No.: 22, 24, 26, 28, 30, 32, 34, 36, 38 and 40;

wherein the promoter is operatively linked to the at least one native endogenous genes.

The plant produced by the method of the invention is characterized by one or more phenotypic features selected from the group consisting of:

delayed senescence; increased vegetative growth; increased biomass production; increased seed production; increased seed lipid content; increased pathogen resistance; increased oxidative stress resistance; wherein the one or more phenotypic features are as compared with a corresponding wild type plant of the same species.

The invention further provides for the use of one or more transgenes encoding an autophagy related ATG5 and/or ATG7 protein for enhancing the productivity of a plant, wherein
the amino acid sequence of the ATG 5 protein has at least 80% amino acid sequence identity to a sequence selected from the group: SEQ ID No.: 2, 4, 6, 8, 10, 12, 14, 16, 18, and 20; and
the amino acid sequence of the ATG 7 protein has at least 80% amino acid sequence identity to a sequence selected from the group: SEQ ID No.: 22, 24, 26, 28, 30, 32, 34, 36, 38 and 40;

wherein the one or more transgene comprises a promoter operatively linked to a coding sequence encoding the protein.

The use of the one or more transgenes according to the invention, wherein the plants (resulting from the use of the one or more transgenes) are characterized by one or more phenotypic features selected from the group consisting of: delayed senescence; increased vegetative growth; increased biomass production; increased seed production; increased seed lipid content; increased pathogen resistance; increased oxidative stress resistance; wherein the one or more phenotypic features are as compared with a corresponding wild type plant of the same species.

According to one embodiment of the invention, the promoter is a constitutive promoter; or a seed-specific promoter, such as a napin promoter; or a modified promoter, where the modification is done by TALENs or CRISPR.

According to one embodiment, the method for enhancing the productivity of a plant by genetic modification makes use of a constitutive promoter; or a seed-specific promoter, such as a napin promoter; or a modified promoter, where the modification is done by TALENs or CRISPR.

In one aspect, the invention relates to the use of the one or more transgenes according to the invention, wherein the plants (resulting from the use of the one or more transgenes) are characterized by one or more transgenes encoding an autophagy related ATG5 and/or ATG7 protein for enhancing the productivity of a plant.

In one embodiment of this aspect, the promoter might be a constitutive promoter; or a seed-specific promoter, such as a napin promoter; or a modified promoter, where the modification is done by TALENs or CRISPR.

### DESCRIPTION OF THE INVENTION

### Figures

FIG. 1. Histogram showing the qRT-PCR quantitation of ATG5 and ATG7 transcripts in wild type (WT) and ATG-overexpressing plants. Representative qRT-PCR performed on cDNA obtained from ten-day old WT, ATG5-overexpressing (ATG5 OE) or ATG7-overexpressing (ATG7 OE) seedlings grown under normal conditions (150 μE m⁻² s⁻¹ light, 16 h photoperiod). Data represents the mean±SD values for individual plants normalized to two reference genes (PP2A and HEL) and to WT; n=3, technical replicates; *, P<0.0001; vs WT, using Dunnett's test.

FIG. 2. Constitutive overexpression of ATG5 or ATG7 stimulates autophagic flux in plants. (a) Image of a Coumassie stained SDS-PAGE (lower panels) and its corresponding western blot (upper panels) of protein samples derived from seven-day-old seedlings of Col-0 (WT), ATG5-overexpressing (ATG5 OE) or ATG7-overexpressing (ATG7 OE) genotypes, following their incubation for 3 days under 150 μE m⁻² s⁻¹ light, 16 h photoperiod (Light) or in the darkness (Dark), and prepared as follows. The seedlings were harvested, total proteins isolated and subjected to ultracentrifugation to yield S; supernatant of 100,000 g fraction, M; pellet of 100,000 g fraction, containing membranes. The two fractions and the original crude extract, C, were analysed by SDS-PAGE; and the total protein loading was visualized by Coomassie brilliant blue staining. Free (*) and lipidated (**) forms of Atg8 were immune-detected on the Western blot.

(b) Image of a Coumassie stained SDS-PAGE (lower panels) and its corresponding western blot (upper panels) of protein samples derived from Col-0 (WT), ATG5-overexpressing (ATG5 OE) or ATG7-overexpressing (ATG7 OE) plants following growth under 150 μE m⁻² s⁻¹, 16 h photoperiod. Rosette leaves were sampled at the onset of flowering (0 days after flowering, DAF) and after 10 days (10 DAF). Total protein extracts from sampled leaves were analysed by SDS-PAGE; and the total protein loading was visualized by the Coomassie brilliant blue staining and used to normalize the amount of NBR1 protein in the samples. Decrease of NBR1 protein for each plant is expressed as % of levels detected at 0 DAF.

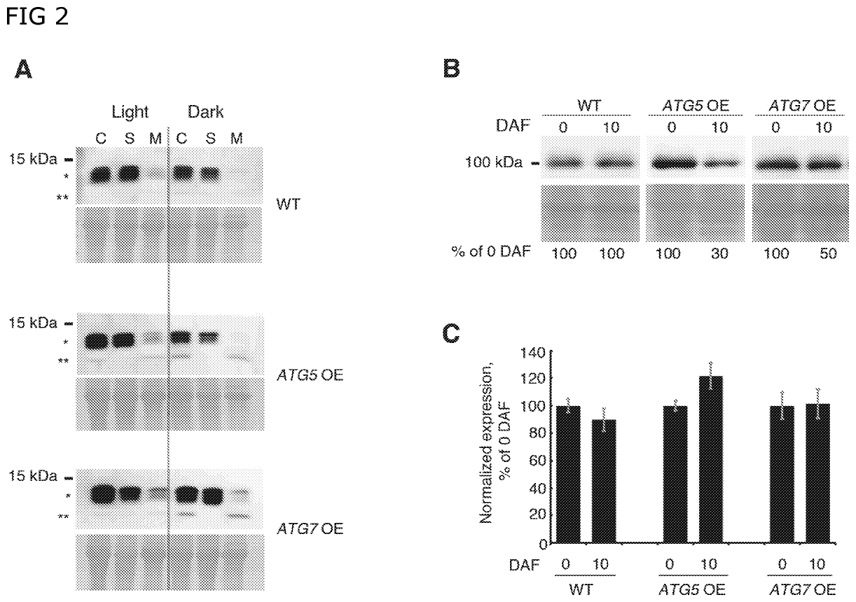

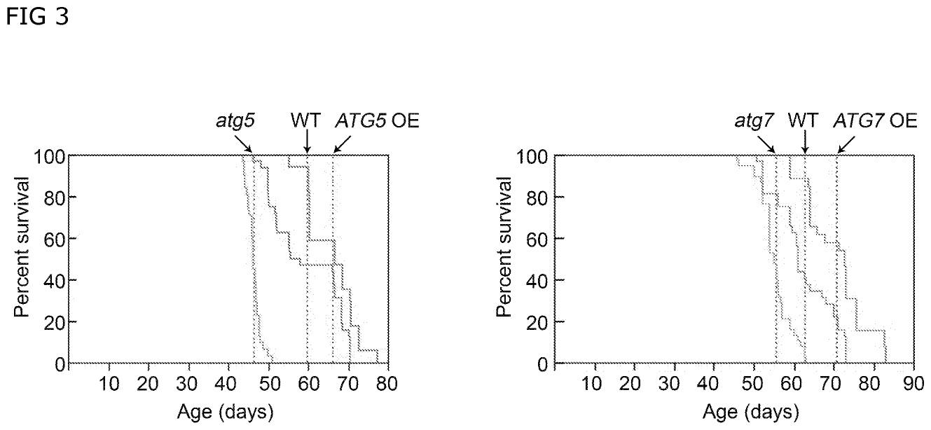

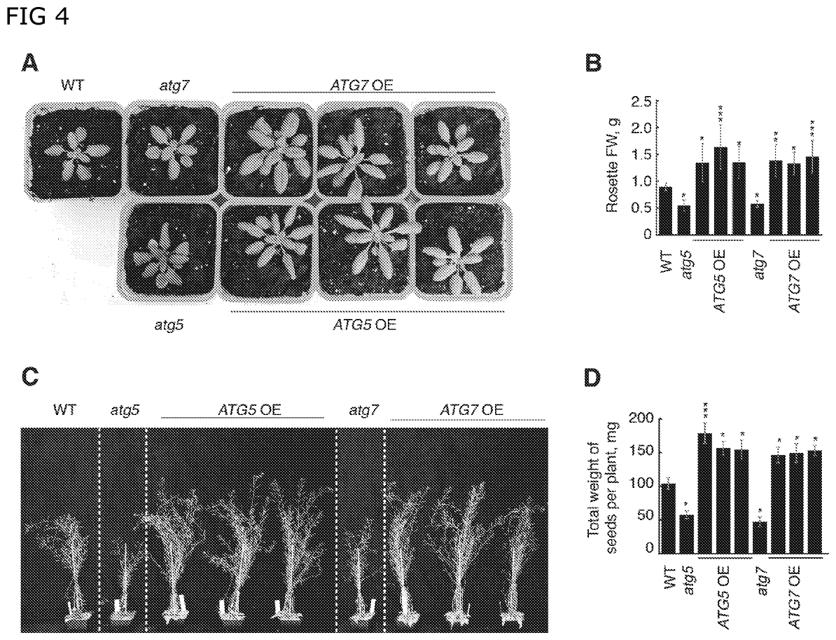

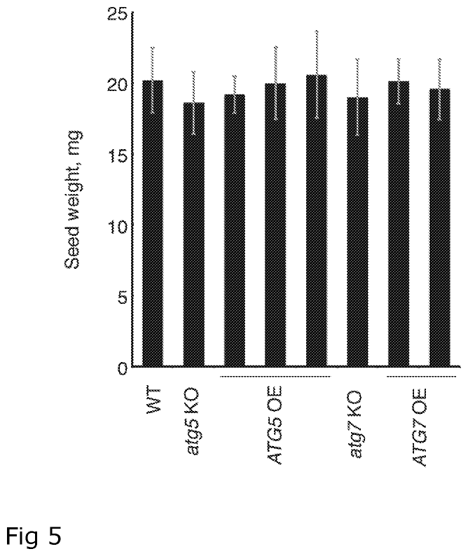

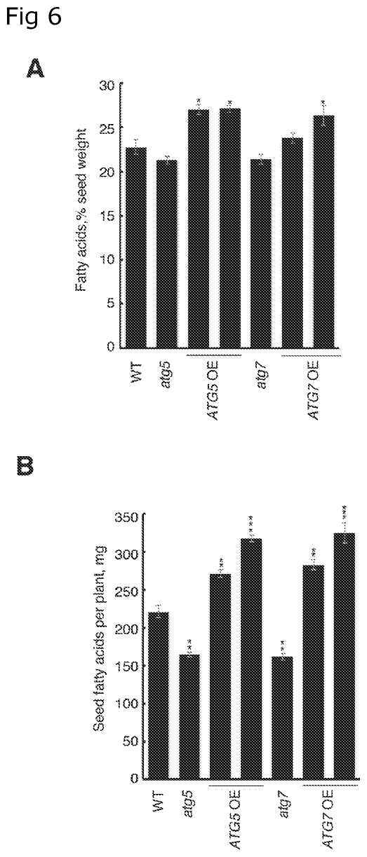

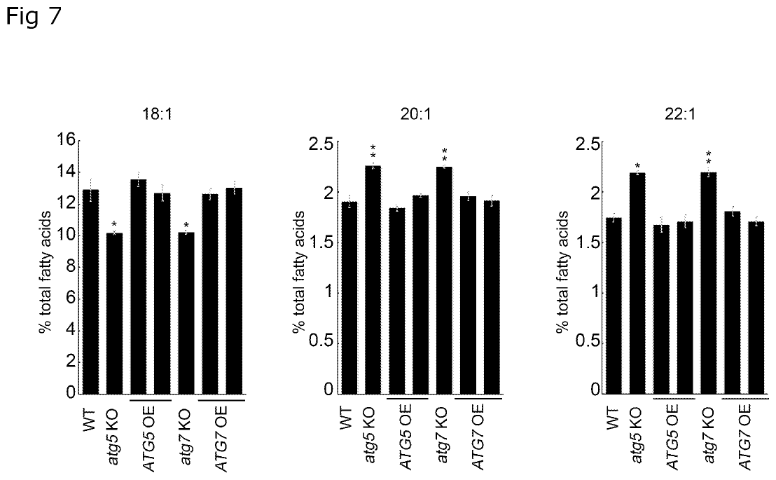

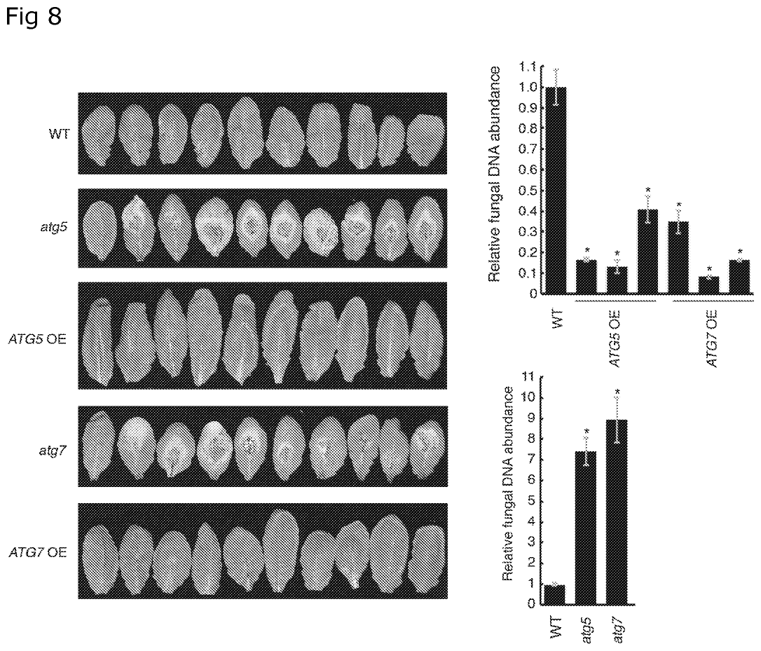

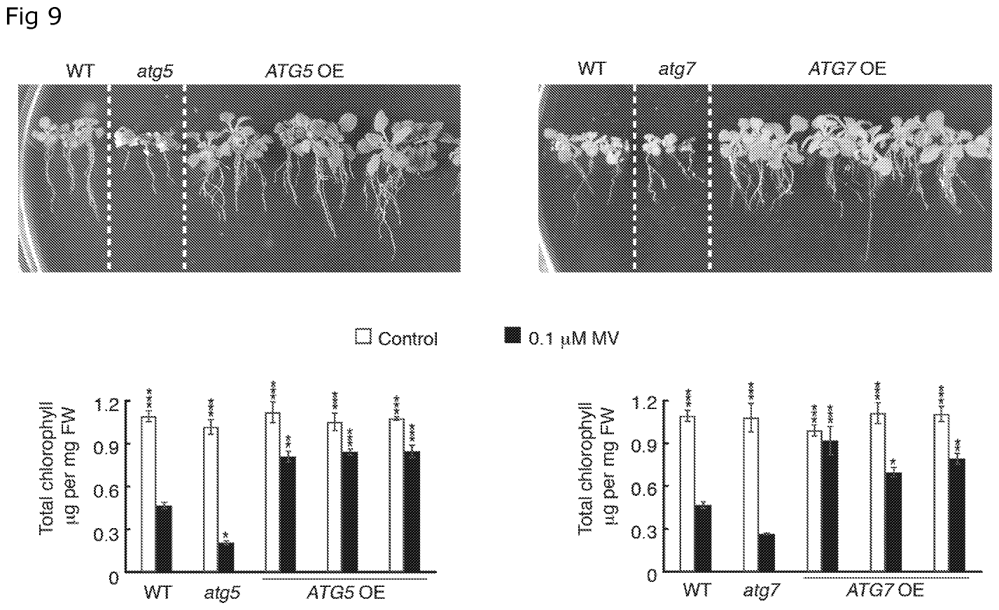

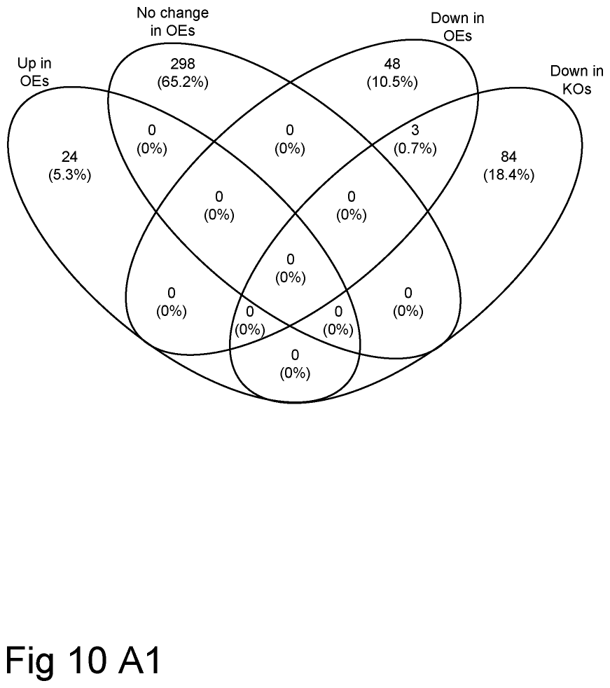

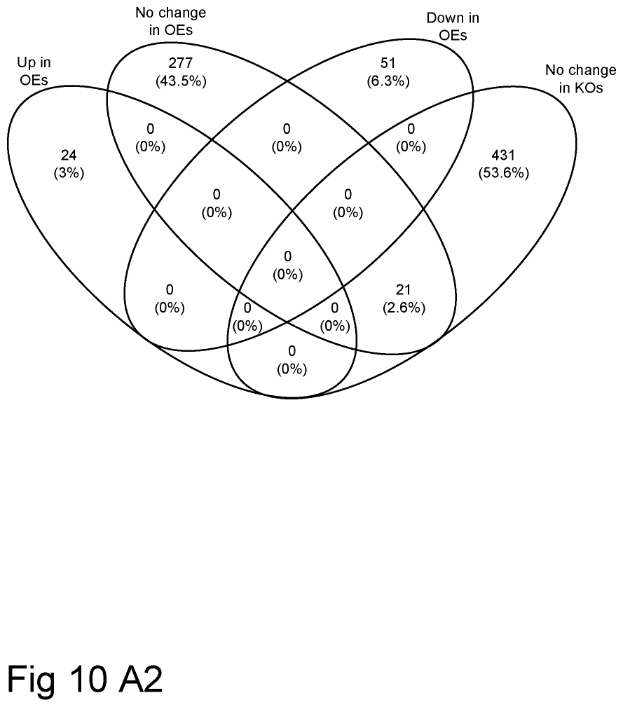

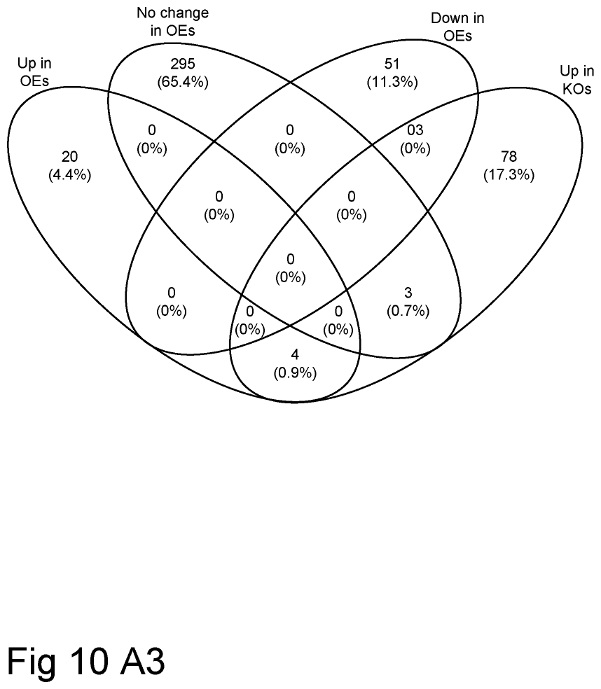

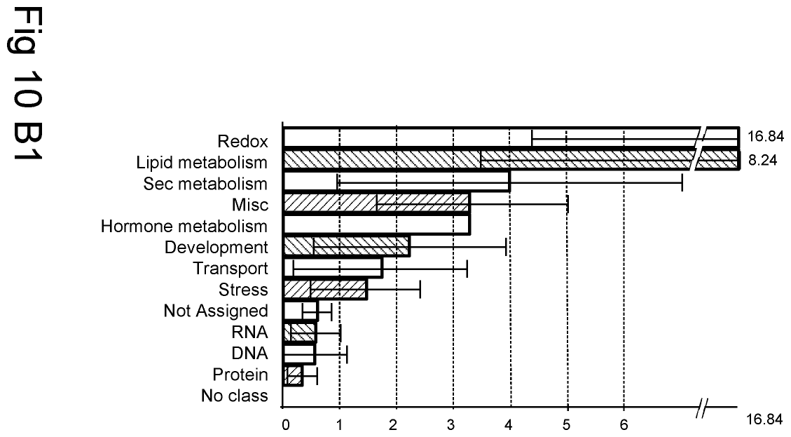

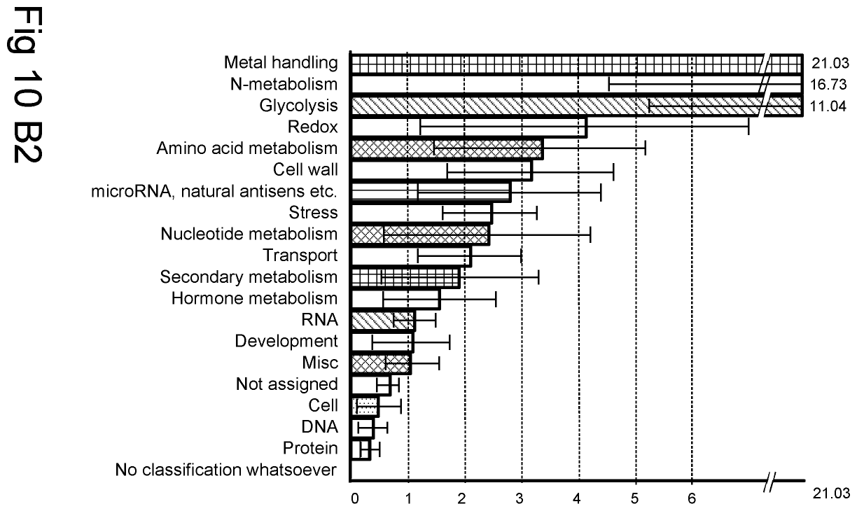

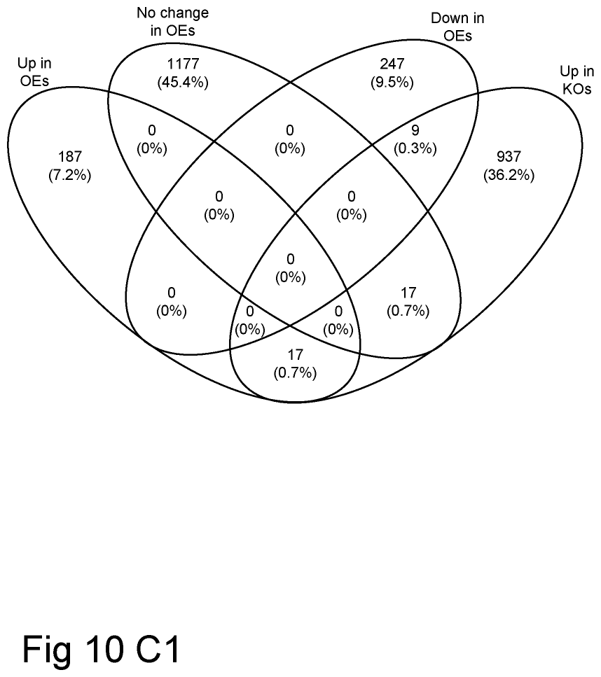

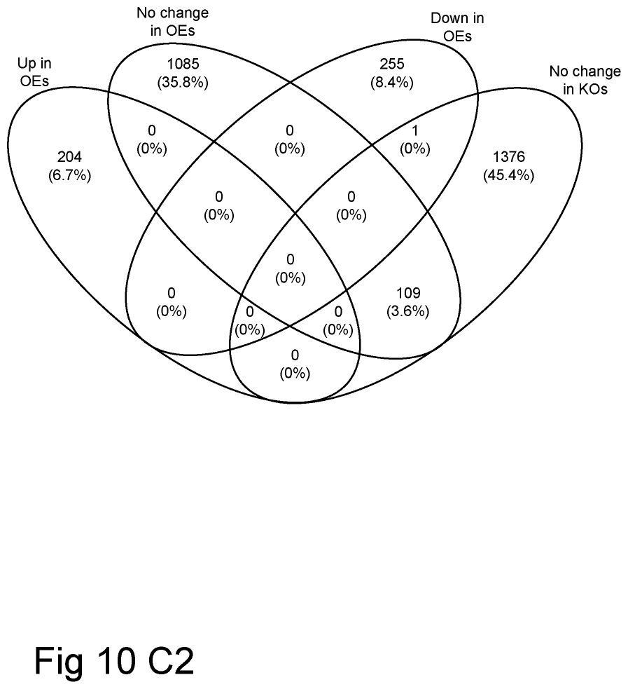

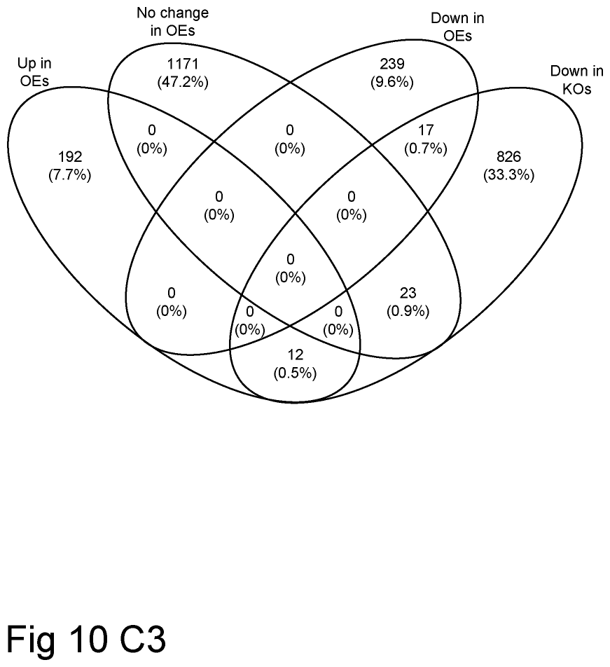

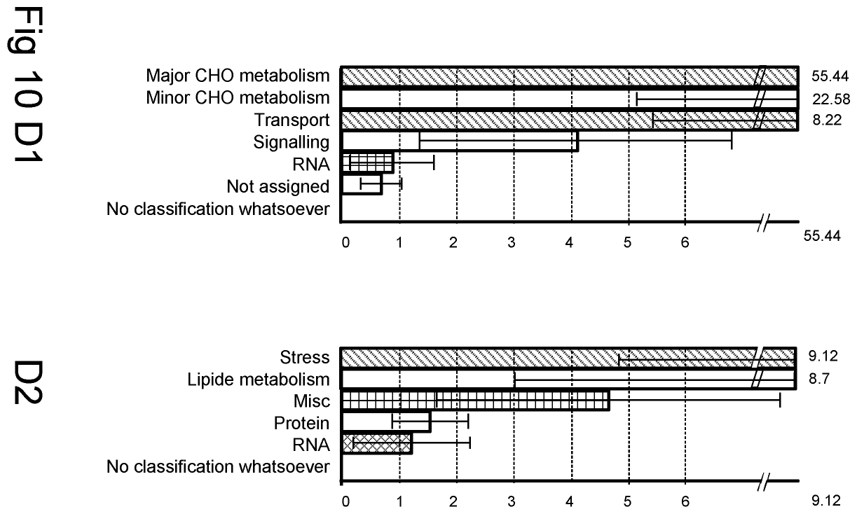

(c) Histogram showing the qRT-PCR quantitation of NBR1 gene transcripts in wild type A. thaliana Col-0 (WT, Col-0) and ATG-overexpressing plants. qRT:PCR was performed on (WT, Col-0), ATG5-overexpressing (ATG5 OE) or ATG7-overexpressing (ATG7 OE) plants following growth under 150 μE m⁻² s⁻¹, 16 h photoperiod; sampling the same leaf material as used for NBR1 protein detection (FIG. 2 (b)). Rosette leaves were sampled at the onset of flowering (0 days after flowering, DAF) and after 10 days (10 DAF). Data represents the mean±SD values for individual plants normalized to two reference genes (PP2A and HEL) and to the 0 DAF; n=3, technical replicates; *, P<0.0001; vs WT, using Dunnett's test.

FIG. 3 Graph showing Kaplan-Meier survival curves for the wild type A. thaliana Col-0 (WT, Col-0) plants, ATG-knockout A. thaliana mutants (atg5 and atg7) and ATG-overexpressing lines (ATG5 OE and ATG7 OE) grown under normal conditions (150 μE m⁻² s⁻¹ light, 16 h photoperiod). The dashed vertical lines show mean lifespans for different genotypes. Each of the two trials was repeated twice, every time with a different ATG5- or ATG7-overexpressing line. The lifespan of an individual plant was measured as the time period from the radicle emergence to complete senescence of rosette and cessation of flowering.

FIG. 4. Illustration of vegetative growth and seed production.

(A) Photographic image of three-week-old plants typical of wild type A. thaliana Col-0 (WT, Col-0) plants, ATG-knockout A. thaliana mutants (atg5 and atg7) and three individual ATG5- or ATG7-overexpressing transgenic A. thaliana lines grown under normal conditions (150 μE m⁻² s⁻¹ light, 16 h photoperiod). (B) Histogram showing plant biomass based on fresh weight of rosette. Data represent mean±SEM, n=3-4. ***: P<0.0001; **: P<0.001; *: P<0.05; vs control (WT), Dunnett's test. (C) Photographic image illustrating the phenotype of plants typical of the same genotypes as in (a) at the flowering stage. (D) Histogram showing total weight of seeds harvested from WT, Col-0, ATG-knockout and ATG5- or ATG7-overexpressing transgenic A. thaliana plants grown under normal conditions. Data represents the mean±SEM, n=6-11. ***, P<0.0001; **, P<0.001; *, P<0.05; vs WT, Col-0, using Dunnett's test.

FIG. 5. Histogram showing the weight of individual seeds harvested from wild type A. thaliana Col-0 (WT, Col-0) plants; ATG-knockout A. thaliana mutants (atg5 and atg7); and ATG5- or ATG7-overexpressing transgenic A. thaliana lines grown under normal conditions. Data represents the mean±SD for at least 9 plants. *: P<0.05; **: P<0.01 vs WT, using Dunnett's test.

FIG. 6. Histogram showing fatty acid content of mature seeds harvested from each of three plants of wild type A. thaliana Col-0 (WT, Col-0) plants, ATG-knockout A. thaliana mutants (atg5 and atg7) and ATG5- or ATG7-overexpressing transgenic A. thaliana lines grown under normal conditions. Fatty acid content is expressed as % of seed weight; and in mg per plant. Data represent mean±SEM, n=3. ***: P<0.001; **: P<0.01; *: P<0.05 vs WT, Col-0, using Dunnett's test.

FIG. 7. Histogram showing 18:1, 20:1 and 22:2 fatty acid content of mature seeds harvested from each of three plants of wild type A. thaliana Col-0 (WT, Col-0) plants, ATG-knockout A. thaliana mutants (atg5 and atg7) and ATG5- or ATG7-overexpressing transgenic A. thaliana lines grown under normal conditions. Individual fatty acid content (18:1, oleic acid; 20:1, eicosa-13,16-dienoic acid; 22:1, erucic acid) is expressed as % total fatty acid extracted from seeds. Data represent mean±SEM, n=3. ***: P<0.001; **: P<0.01 vs WT, using Dunnett's test.

FIG. 8. Illustration of A. thaliana plants infected with Alternaria brassicicola. Three-week-old wild type A. thaliana Col-0 (WT, Col-0) plants, ATG-knockout A. thaliana mutants (atg5 and atg7) and ATG5- or ATG7-overexpressing transgenic A. thaliana lines were inoculated with 10 μL of suspension containing 5×10⁵ spores mL⁻¹ of Alternaria brassicicola. Photographic images show rosette leaves from the respective infected plants on the 7th day post inoculation. Histograms show the fungal growth on each of the respective infected plants assessed by measuring fungal DNA using qRT-PCR to detect the fungal cutinase gene. Data represents the mean±SEM normalized to two reference genes (UBQ5 and PR), n≥3. ****: P<0.0001; *: P<0.05 vs WT, using Dunnett's test.

FIG. 9. Photographic image of A. thaliana plants following exposure to oxidative stess. Seeds of wild type A. thaliana Col-0 (WT, Col-0) plants, ATG-knockout A. thaliana mutants (atg5 and atg7) and ATG5- or ATG7-overexpressing transgenic A. thaliana lines were germinated on MS plates with or without addition of 0.1 μM methyl viologen (MV), and their phenotype after 3 weeks of growth is shown. The histogram shows the measured chlorophyll content of untreated plants (control) and plants treated with MV. Data represents the mean±SEM, n=3-7. ***: P<0.0001; **: P<0.001; *: P<0.05 vs MV-treated WT, using Dunnett's test.

FIG. 10A. Comparison of transcriptomics profiles revealed strong trends specific for ATG-overexpressing and ATG-deficient plants. A1, A2 and A3: Venn diagrams visualising number of transcripts down-regulated, showing no change, or up-regulated in ATG-overexpressing or ATG-depleted backgrounds compared to WT at the budding stage.

FIG. 10B. Gene ontology of transcripts up-regulated in both ATG over-expressing or both atg knockout genotypes at the budding stage. B1; Transcripts up-regulated in ATG-over-expressors only, B2 Transcripts up-regulated in atg knockouts only.

FIG. 10C 1, 2 and 3. Venn diagrams visualising number of transcripts down-regulated, showing no change, or up-regulated in ATG-overexpressing or ATG-depleted backgrounds compared to WT at the 10 DAF.

FIG. 10D. Gene ontology of transcripts with opposite transcriptional profiles in in ATG-over-expressing and atg knockout genotypes at the 10 DAF. 10D1 Transcripts up-regulated in ATG-over-expressers and down-regulated in atg knockouts. 10D2 Transcripts down-regulated in ATG-over-expressers and up-regulated in atg knockouts.

### ABBREVIATIONS AND TERMS

gi number: (genInfo identifier) is a unique integer which identifies a particular sequence, independent of the database source, which is assigned by NCBI to all sequences processed into Entrez, including nucleotide sequences from DDBJ/EMBL/GenBank, protein sequences from SWISS-PROT, PIR, Phytozome for plant specific sequences and many others.

Amino acid sequence identity: The term “sequence identity” as used herein, indicates a quantitative measure of the degree of homology between two amino acid sequences of substantially equal length. The two sequences to be compared must be aligned to give a best possible fit, by means of the insertion of gaps or alternatively, truncation at the ends of the protein sequences. The sequence identity of the polypeptides of the invention can be calculated as (Nref−Ndif)100/Nref, wherein Ndif is the total number of non-identical residues in the two sequences when aligned and wherein Nref is the number of residues in one of the sequences. The sequence identity between one or more sequence may also be based on alignments using the clustalW or ClustalX software. In one embodiment of the invention, alignment is performed with the sequence alignment method ClustalX version 2 with default parameters. The parameter set preferably used are for pairwise alignment: Gap open penalty: 10; Gap Extension Penalty: 0.1, for multiple alignment, Gap open penalty is 10 and Gap Extension Penalty is 0.2. Protein Weight matrix is set on Identity. Both Residue-specific and Hydrophobic Penalties are “ON”, Gap separation distance is 4 and End Gap separation is “OFF”, No Use negative matrix and finally the Delay Divergent Cut-off is set to 30%.

Preferably, the numbers of substitutions, insertions, additions or deletions of one or more amino acid residues in the polypeptide as compared to its comparator polypeptide is limited, i.e. no more than 1, 2, 3, 4, 5, 6, 7, 8, 9, or 10 substitutions, no more than 1, 2, 3, 4, 5, 6, 7, 8, 9, or 10 insertions, no more than 1, 2, 3, 4, 5, 6, 7, 8, 9, or 10 additions, and no more than 1, 2, 3, 4, 5, 6, 7, 8, 9, or 10 deletions. Preferably the substitutions are conservative amino acid substitutions: limited to exchanges within members of group 1: Glycine, Alanine, Valine, Leucine, Isoleucine; group 2: Serine, Cysteine, Selenocysteine, Threonine, Methionine; group 3: Proline; group 4: Phenylalanine, Tyrosine, Tryptophan; Group 5: Aspartate, Glutamate, Asparagine, Glutamine.

Operably linked: a gene (nucleic acid molecule comprising a coding sequence) is operably linked to a promoter when its transcription is under the control of the promoter and where transcription results in a transcript whose subsequent translation yields the product encoded by the gene.

The term “increasing expression” is intended to encompass well known methods to increase the expression by regulatory sequences, such as promoters, or proteins, such as transcription factors. The terms “increasing expression”, “enhanced expression” and “over-expression” can be used interchangeably in this text. Increased expression may lead to an increased amount of the over-expressed protein/enzyme, which may lead to an increased activity of the protein of interest that contributes to its high efficiency.

The term “enhanced productivity” is intended to encompass the productivity of a plant is the sum of several important traits that determine the rate of generation of biomass by the plant in an ecosystem. The key traits of a genetically modified plant of the invention, that contribute to its high productivity include: delayed aging, enhanced vegetative growth and seed production, increased accumulation of storage lipids (including seed storage lipids); enhanced pathogen resistance and enhanced oxidative stress resistance.

### DETAILED DESCRIPTION OF THE INVENTION

The present invention provides a method for enhancing the productivity of a plant by genetically modifying the genome of the plant to over-express at least one autophagy-related (ATG) protein selected from the group consisting of ATG5 and ATG7. The invention further provides a genetically modified plant characterized by over-expression of least one autophagy-related (ATG) protein selected from the group consisting of ATG5 and ATG7.

I A Genetically Modified Plant Characterized by Over-Expression of an Autophagy-Related (ATG) Protein

The genetically modified plant of the invention is characterized by over-expression of an autophagy-related (ATG) protein selected from the group consisting of ATG5 and/or ATG7. The ATG 5 protein, overexpressed in a plant of the invention, is functionally characterized by the ability to enhance the productivity of the plant.

The structural and functional domains of the ATG5 protein, underlying its ability to enhance plant productivity, are as follows. ATG5 is a structural protein consisting of an N-terminal α-helix domain and two ubiquitin-like domains (Ub1A and ub1B) that flank a central α-helical bundle region (HBR) (FIG. 10). In vivo, the amino acid residue Lys149, located in the HR domain, is used in the conjugation of ATG5 to ATG12. The ATG12-ATG5 conjugate exhibits E3 ligase-like activity which facilitates the lipidation of ubiquitin-like proteins of the LC3 family, which is an essential step in autophagosome formation.

The structure of each of the at least four domains in the ATG 5 protein, overexpressed in a plant of the invention, are characterized as follows:

The N-terminal α-helix domain of ATG 5, which lies adjacent to Ub1A domain, comprises at least two consecutive hydrophobic amino acids (such as valine and tryptophan at positions 9 and 10 of SEQ ID No.: 2), that interact with hydrophobic residues in the Ub1B and the HR domain respectively. In this manner, the α-helix domain plays a role in the assembly and architecture of ATG 5.

The Ub1A and Ubi1B domains each comprise 5 β-sheets and 2 α-helices. The amino acid sequence of the Ub1A domain of ATG 5 has at least 70, 72, 74, 76, 78, 80, 82, 84, 86, 88, 90, 92, 94, 96, 98, 99 and 100% amino acid sequence identity with the sequence of residues 12-104 of SEQ ID No 2. The amino acid sequence of the Ub1B domain (corresponding to residues 210-332 of SEQ ID No 2) is less highly conserved (see Example 6).

The HR domain of ATG5 is a helix-rich domain comprising three long and one short α-helix. The amino acid sequence of the HR domain of ATG 5 has at least 60, 62, 64, 66, 68, 70, 72, 74, 76, 78, 80, 82, 84, 86, 88, 90, 92, 94, 96, 98, 99 or 100% amino acid sequence identity with the sequence of residues 118-173 of SEQ ID No 2; with the proviso that the residue corresponding to amino acid 128 in SEQ ID No: 2 is lysine (required for the conjugation of ATG5 with ATG12).

Linker domains serve to link the HR domain to the flanking Ub1A and Ub1B domains. Linker 1, between Ub1A and HR, is characterized by at least three hydrophobic residues (two or more of valine, leucine, isoleucine and proline); that serve to interact with and fix the spacial arrangement of Ub1A—HR. The amino acid sequence of the linker 1 domain of ATG 5 has at least 62, 64, 66, 68, 70, 72, 74, 76, 78, 80, 82, 84, 86, 88, 90, 92, 94, 96, 98, 99 or 100% amino acid sequence identity with the sequence of residues 104-118 of SEQ ID No 2.

The amino acid sequence of the ATG5 polypeptide has at least 52, 54, 58, 60, 62, 64, 66, 68, 70, 72, 74, 76, 78, 80, 82, 84, 86, 88, 90, 92, 94, 96, 98, 99 or 100% amino acid sequence identity to SEQ ID No.: 2. In one embodiment the amino acid sequence of the ATG5 polypeptide has at least 80, 82, 84, 86, 88, 90, 92, 94, 96, 98, 99 or 100% sequence identity to a sequence selected from the group consisting of: SEQ ID No.: 2, 4, 6, 8, 10, 12, 14, 16, 18 and 20.

The ATG protein that is over-expressed in a genetically modified plant of the invention may alternatively be ATG7. An ATG 7 protein, overexpressed in a plant of the invention, is functionally characterized by the ability to enhance the productivity of the plant.

The structural and functional domains of the ATG7 protein, underlying its ability to enhance plant productivity, are as follows. ATG7 is a structural protein consisting of an N-Terminal Domain (NTD); an Adenylation Domain (AD); and an Extreme C-Terminal Domain (ECTD) domain ending in a C-terminal tail. ATG7 is an E1 enzyme, that in vivo acts as a dimer, and activates the ubiquitin-like proteins ATG8 and ATG12, and transfers them to their cognate E2 enzymes, ATG3 and ATG10 respectively.

The structure of each of the at least three domains in the ATG 7 protein, overexpressed in a plant of the invention, are characterized as follows:

The NTD domain comprises six α-helices and 15 β-strands; and interacts with ATG3. The amino acid sequence of the NTD domain of ATG 7 has at least 45, 50, 55, 60, 65, 70, 72, 74, 76, 78, 80, 82, 84, 86, 88, 90, 92, 94, 96, 98, 99 or 100% amino acid sequence identity with the sequence of residues 11-320 of SEQ ID No 22.

The AD domain comprising seven α-helices and 10 β-strands; and interacts with the ATG8. A catalytic cysteine, at position 507 within the AD domain activates and forms a thioester conjugate with ATG8; which is then transferred to ATG3 bound to the NTD domain. The two arginine residues (R1 R2 in FIG. 11) are essential for ATG8-PE conjugate formation. The amino acid sequence of the AD domain of ATG 7 has at least 45, 50, 55, 60, 65, 70, 72, 74, 76, 78, 80, 82, 84, 86, 88, 90, 92, 94, 96, 98, 99 or 100% amino acid sequence identity with the sequence of residues 327-637 of SEQ ID No 22.

The ECTD essential for an initial interaction of ATG7 with ATG8; where ATG8 is then transferred to the AD domain. The amino acid sequence of the ECTD domain of ATG 7 has at least 55, 60, 65, 70, 72, 74, 76, 78, 80, 82, 84, 86, 88, 90, 92, 94, 96, 98, 99 or 100% amino acid sequence identity with the sequence of residues 638-678 of SEQ ID No 22.

The amino acid sequence of the ATG7 polypeptide has at least 54, 58, 60, 62, 64, 66, 68, 70, 72, 74, 76, 78, 80, 82, 84, 86, 88, 90, 92, 94, 96, 98, 99 or 100% amino acid sequence identity to SEQ ID No.: 22. In one embodiment the amino acid sequence of the ATG7 polypeptide has at least 80, 82, 84, 86, 88, 90, 92, 94, 96, 98, 99 or 100% sequence identity to a sequence selected from the group consisting of: SEQ ID No.: 22, 24, 26, 28, 30, 32, 34, 36, 38 and 40.

In one embodiment of the genetically modified plant of the invention, the coding sequence of at least one native endogenous gene(s) encoding the ATG5 and/or ATG7 protein is operably linked to a constitutive promoter that drives constitutive expression of the cognate native endogenous gene encoding the ATG5 and/or ATG7 protein.

In an alternative embodiment, the genetically modified plant of the invention, comprises at least one transgene(s) encoding the ATG5 and/or ATG7 protein, where the coding region of the at least one transgene is operably linked to a constitutive promoter that drives constitutive expression of the cognate transgene encoding the ATG5 and/or ATG7 protein.

The genetically modified plant of the invention comprises at least one transgene(s) or at least one native gene(s) encoding the ATG5 and/or ATG7 protein; wherein the expression of ATG5 and/or ATG7 protein is constitutive. The constitutive promoter driving constitutive expression of ATG5 and/or ATG7 protein may for example be selected from CaMV 35S promoter (SEQ ID No.: 66 or the following promoters; opine gene promoter, and mannopine synthase (mas) promoter; cassava vein mosaic virus (CsVMV) promoter, and the alfalfa small subunit Rubisco (RbcS) promoter; PtMCP promoter.

The genetically modified plant of the invention comprising at least one transgene(s) encoding the ATG5 and/or ATG7 protein further comprises a transcription termination sequence (e.g. nopaline synthase (nos) terminator sequence (SEQ ID No.: 61)).

II A Genetically Modified Plant Over-Expressing Autophagy-Related ATG5 and/or ATG 7 Proteins is Characterized by Enhanced Productivity

A genetically modified plant of the invention, that over-expresses autophagy-related ATG5 and/or ATG 7 proteins, is characterized by enhanced productivity. The productivity of a crop plant is the sum of several agronomically important traits that determine the rate of generation of biomass by the plant in an ecosystem. The key agronomic traits of the genetically modified plant of the invention, that contribute to its high productivity include: delayed aging, enhanced vegetative growth and seed production, increased accumulation of storage lipids (including seed storage lipids); enhanced pathogen resistance and enhanced oxidative stress resistance.

Ii Delayed Senescence

A genetically modified plant of the invention is characterized by a longer life-span, including an extended flowering period (Example 2) as compared with a corresponding wild type plant. In particular, overexpression of either the ATG5 or 7 proteins in genetically modified plants causes a significantly delay in the onset of leaf senescence without affecting the duration of leaf senescence. The total lifespan of the genetically modified plants may be increased, for example by 10% to 20% as compared with a corresponding wild type plant. The productivity of a genetically modified plant of the invention is enhanced by the increase in life span before the onset of leaf senescence, since this extends the period for photosynthetic assimilation of biomass.

Iii Enhanced Vegetative Growth and Seed Production

A genetically modified plant of the invention is characterized by an increase in vegetative growth (Example 3) as compared with a corresponding wild type plant. When the genetically modified plant of the invention produces seeds, the yield of seeds is typically increased due to the plant's increased fecundity (seed set), which is correlated with the extended duration of flowering in the genetically modified plant. An increase in seed yield in the genetically modified plant is not at the expense of individual seed weight, which is not significantly different from a corresponding wild type plant (Example 3).

Iiii Increased Accumulation of Lipid Assimilation

When the genetically modified plant of the invention produces seeds, the oil content of the seeds is typically increased as compared with a corresponding wild type plant (Example 4). Since the yield of seeds produced by the genetically modified plant of the invention is typically increased, the total yield of seed oil (fatty acid) per plant is increased, typically in the range of 25% to 50% increase as compared to a corresponding wild type plant.

When the genetically modified plant of the invention produced seeds comprising oil (triacylglycerol) reserves, the increase in seed oil assimilation as compared to a corresponding wild type plant is an important agronomic property.

Iiv Enhanced Pathogen Resistance

A genetically modified plant of the invention is characterized by an increased pathogen resistance as compared with a corresponding wild type plant. Enhanced resistance to necrotrophic fungal pathogens in the genetically modified plant is characterized by fewer necrotic lesions and suppressed fungal growth as compared to a corresponding wild type plant (Example 5). An enhanced ability to contain or limit pathogen growth in the plants is a key parameter for enhancing the agronomic performance and eventual yield of the plants of the invention.

Iv Enhanced Oxidative Stress Resistance.

A genetically modified plant of the invention is characterized by an increased oxidative stress resistance as compared with a corresponding wild type plant. One of the major components of necrotrophic pathogenicity is oxidative stress. Enhanced autophagy in the genetically modified plant of the invention enables a more effective reallocation of limited resources from growth to stress resistance (and vice versa) so as to reduce the fitness costs required for survival under adverse environmental conditions.

Autophagy, in general, is known to participate in the recycling of chloroplastic proteins and whole chloroplasts in leaves, thus supporting nitrogen remobilization and nitrogen use efficiency. While not wishing to be bound by theory, it is likely that more efficient flux of nitrogen from source to sink will enhance flowering and increase seed set, both traits being consistently observed in the transgenic plants with enhanced autophagy (Example 3).

III A Genetically Modified Plant Cell, Plant or a Part Thereof According to the Invention that has Increased Productivity

A genetically-modified or transgenic plant cell or plant or a part thereof according to the present invention, that over-expresses autophagy related ATG5 and/or ATG 7 proteins, may be an annual plant or a perennial plant.

Preferably the annual or perennial plant is a crop plant having agronomic importance. The annual crop plant can be a monocot plant selected from Avena spp (Avena sativa); Oryza spp., (e.g. Oryza sativa; Oryza bicolour); Hordeum spp., (Hordeum vulgare); Triticum spp., (e.g. Triticum aestivum); Secale spp., (Secale cereale); Brachypodium spp., (e.g. Brachypodium distachyon); Zea spp (e.g. Zea mays); or a dicot plant selected from Cucumis spp., (e.g. Cucumis sativus); Glycine spp., (e.g. Glycine max); Medicago spp., (e.g. Medicago trunculata); Mimulus spp; Brassica spp (e.g. Brassica rapa; Brassica napus; Brassica oleraceae); Camelina spp (e.g. Camelina sativa); Beta vulgaris. Preferably the perennial plant is a woody plant or a woody species. The woody plant may be a hardwood plant e.g. selected from the group consisting of acacia, eucalyptus, hornbeam, beech, mahogany, walnut, oak, ash, willow, hickory, birch, chestnut, poplar, alder, maple, sycamore, ginkgo, a palm tree and sweet gum. Hardwood plants, such as eucalyptus and plants from the Salicaceae family, such as willow, poplar and aspen including variants thereof, are of particular interest, as these groups include fast-growing species of tree or woody shrub which are grown specifically to provide timber for building material, raw material for pulping, bio-fuels and/or bio chemicals.

In further embodiments, the woody plant is a conifer which may be selected from the group consisting of cypress, Douglas fir, fir, sequoia, hemlock, cedar, juniper, larch, pine, redwood, spruce and yew.

In other embodiments, the woody plant is a fruit bearing plant which may be selected from the group consisting of apple, plum, pear, banana, orange, kiwi, lemon, cherry, grapevine, papaya, peanut, and fig.

Alternatively, the woody plants which may be selected from the group consisting of cotton, bamboo and rubber plants.

The present invention extends to any plant cell of the above genetically modified, or transgenic plants obtained by the methods described herein, and to all plant parts, including harvestable parts of a plant, seeds, somatic embryos and propagules thereof, and plant explant or plant tissue. The present invention also encompasses a plant, a part thereof, a plant cell or a plant progeny comprising a DNA construct according to the invention. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced in the parent by the methods according to the invention. It should be noted that embodiments and features described in the context of one of the aspects of the present invention also apply to the other aspects of the invention. Thus, definitions of one embodiment regard mutatis mutandis to all other embodiments comprising or relating to the one embodiment. When for example definitions are made regarding DNA constructs or sequences, such definitions also apply with respect to methods for producing a plant, vectors, plant cells, plants comprising the DNA construct and vice versa. A DNA construct described in relation to a plant also regards all other embodiments

IV A Method for Enhancing the Productivity of a Plant by Genetic Modification

One or more transgenes encoding an ATG5 and/or an ATG 7 protein; wherein the transgene is operably linked to a constitutive promoter, may be introduced into a plant cell by transformation.

Transformation of Plant Cells

In accordance with the present invention, the method comprises transforming regenerable cells of a plant with a nucleic acid construct or recombinant DNA construct (as described in I) and regenerating a transgenic plant from said transformed cell. Production of stable, fertile transgenic plants is now a routine method.

Various methods are known for transporting the construct into a cell to be transformed. Agrobacterium-mediated transformation is widely used by those skilled in the art to transform tree species, in particular hardwood species such as poplar and Eucalyptus. Other methods, such as microprojectile or particle bombardment, electroporation, microinjection, direct DNA uptake, liposome mediated DNA uptake, or the vortexing method may be used where Agrobacterium transformation is inefficient or ineffective, for example in some gymnosperm species.

A person of skill in the art will realize that a wide variety of host cells may be employed as recipients for the DNA constructs and vectors according to the invention. Non-limiting examples of host cells include cells in embryonic tissue, callus tissue type I, II, and III, hypocotyls, meristem, root tissue, tissues for expression in phloem, leaf discs, petioles and stem internodes. Once the DNA construct or vector is within the cell, integration into the endogenous genome can occur.

Selection of Transformed Plant Cells and Regeneration of Plant or Woody Plants

Following transformation, transgenic plants are preferably selected using a dominant selectable marker incorporated into the transformation vector. Typically, such a marker will confer antibiotic or herbicide resistance on the transformed plants and selection of transformants can be accomplished by exposing the plants to appropriate concentrations of the antibiotic or herbicide. A selection marker using the D-form of amino acids and based on the fact that plants can only tolerate the L-form offers a fast, efficient and environmentally friendly selection system.

Subsequently, a plant may be regenerated, e.g. from single cells, callus tissue or leaf discs, as is standard in the art. Almost any plant can be entirely regenerated from cells, tissues and organs of the plant. After transformed plants are selected and they are grown to maturity and those plants showing altered growth properties phenotype are identified.

Furthermore, one or more native endogenous ATG 5 and/or ATG7 genes in the plant of the invention may be genetically modified to express elevated levels of ATG5 and/or an ATG 7 proteins; by replacing the endogenous ATG promoter with a strong, constitutively active promoter of another gene (e.g. actin gene promoter) using methods for site-directed mutagenesis such as TALENs or CRISPR.

V Methods for Detecting Modified Expression of a Gene Encoding a Polypeptide in a Plant or Woody Plant of the Invention

Real-time RT-PCR can be used to compare gene expression, i.e. the mRNA expression, levels in a GM plant or woody plant with the corresponding non-GM plant or woody plant. The amount of the polynucleotides disclosed herein can be determined using Northern blots, sequencing, RT-PCR or microarrays.

Western blots with immune detection or gel shift assays can be used to measure the expression levels or amounts of a polypeptide expressed in a GM woody plant of the invention. Antibodies raised to the respective polypeptide may be used for specific immune-detection of the expressed polypeptide in tissue derived from a woody plant.

### EXAMPLES

### Example 1. Genetically Modified Arabidopsis thaliana Over-Expressing a Transgene Encoding ATG5 or ATG7 Enhances Autophagic Flux

A panel of homozygous transgenic lines constitutively overexpressing ATG5 or ATG7 were generated and compared with wild type A. thaliana Col-0 (WT, Col-0) plants.

1.1 Genetic Modification of Arabidopsis thaliana to Generate Homozygous Transgenic Lines Comprising Transgenes Encoding ATG5 or ATG7

Genetically modified Arabidopsis thaliana (A. thaliana) plants comprising transgenes encoding ATG5 or ATG7 were generated as follows: an A. thaliana cDNA library was amplified using primer pairs attB1-ATG5UTR-Fw/attB2-ATG5-Rev and FWatg7/RVatg7 (Table 1), in order to amplify cDNAs encoding the proteins ATG5 and ATG7 respectively. The respective PCR products were individually recombined into a pGWB2 vector (Nakagawa et al., 2007) using Gateway cloning system (Invitrogen) where expression of the inserted ATG5- and ATG7-coding sequences is under the control of the constitutive the cauliflower mosaic virus (CaMV) 35S promoter.

TABLE 1
List of primers

| | | SEQ |
|---|---|---|
| | | ID |
| Primer name | 5′ primer sequence 3′ | NO.: |
| attB1-ATG5UTR- | 5'-GGGGACAAGTTTGTACAAAAAAGC | 41 |
| Fw | AGGCTATACAGAAACAGCGTCGTTTTG-3′ | |
| attB2-ATG5-Rev | 5′-GGGGACCACTTTGTACAAGAAAGCT | 42 |
| | GGGTTCACCTTTGAGGAGCTTTCACAAG-3′ | |
| AtATG5 qPCR | 5′-ATACACTTTAGAGGATATCCTTGCA-3′ | 43 |
| Fw2 | | |
| AtATG5 qPCR | 5′-ACCGTTCATGACAGAGGTCCATA-3′ | 44 |
| Re2 | | |
| AtATG7 qPCR | 5′-TCTAATCCAGTCAGGCAATCTCT-3′ | 45 |
| Fw2 | | |
| AtATG7 qPCR | 5′-GATTCAATCAACTCGCTAAGGCGT-3′ | 46 |
| Re2 | | |
| NBR1 qPCR Fw | 5′-GAGGACCCAGACCGGAAGG-3′ | 47 |
| NBR1 qPCR Re | 5′-GACAAACACGACGAGGATGC-3′ | 48 |
| PP2A qPCR Fw | 5′-TAACGTGGCCAAAATGATGC-3′ | 49 |
| PP2A qPCR Re | 5′-GTTCTCCACAACCGCTTGGT-3′ | 50 |
| HEL qPCR Fw | 5′-CCATTCTACTTTTTGGCGGCT-3' | 51 |
| HEL qPCR Re | 5′-TCAATGGTAACTGATCCACTCTGATG- | 52 |
| | 3′ | |
| FWatg7 | 5′-GGGGACAAGTTTGTACAAAAAAGCAG | 53 |
| | GCTTGATGGCTGAGAAAGAAACTCCA-3′ | |
| RVatg7 | 5′-GGGGACCACTTTGTACAAGAAAGCTGG | 54 |
| | GTATTAAAGATCTACAGCTACATCG-3′ | |
| UBQ 5 FW | 5′-GACGCTTCATCTCGTCC-3′ | 55 |
| UBQ 5 RV | 5′-CCACAGGTTGCGTTAG-3′ | 56 |
| PR2 FW | 5′-AGGAGCTTAGCCTCACCACC-3′ | 57 |
| PR2 RV | 5′-GAGGATGAGCTCGATGTCAGAG-3′ | 58 |
| Cutinase FW | 5′-ATCACTGCCGGTGGTTACTC-3′ | 59 |
| Cutinase FW | 5′-CGACACCCTTGATTTGGTCT-3′ | 60 |

The resulting pGWB2 constructs were transformed into wild type A. thaliana plants of Col-0 ecotype. The plants were transformed with the pGWB2 constructs by means of the Agrobacterium tumifaciens strain GV3101, using the floral dip method (Clough & Bent, 1998). Transgenic plants were selected on MS medium (Murashige and Skoog, 1962) containing 50 μg mL-1 kanamycin.

The genetically modified and wild-type Arabidopsis thaliana plants were cultivated as follows: Seeds of transgenic and control A. thaliana plants were dried at 37° C. for 48 h, treated at −20° C. overnight, surface-sterilized in 15% bleach for 10 min and rinsed in sterile deionized water. Sterilized seeds were placed on half-strength MS medium (supplied by Duchefa, Netherlands), supplemented with 1% (w/v) sucrose, 10 mM MES (pH 5.8) and 0.6% (w/v) plant agar (supplied by Duchefa, Netherlands) and vernalized at 4° C. for 48 h. Germination was carried out in growth rooms at 16 h/8 h light/dark cycles, light intensity 110 μE m⁻² s⁻¹), and 22° C./20° C. day/night temperature. Seedlings with 4 rosette leaves were transferred into individual pots and grown in controlled environment cabinets (Percival AR-41L2, CLF Plant Climatics, Germany) at 16 h/8 h light/dark cycles, at 65% relative humidity, 22° C./20° C. day/night temperature and light intensity adjusted to required level (100 or 150 μE m⁻² s⁻¹) at the level of leaf rosette.

Transgenic plants were propagated as described, and homozygous transgenic seeds selected in the T3 generation were used for further experiments.

1.2 Homozygous Arabidopsis thaliana Lines Comprising ATG5- or ATG7-Encoding Transgenes have Higher Transcript Levels

Transcription of the ATG5- or ATG7-encoding transgenes in transgenic A. thaliana lines was detected by quantitative RT-PCR, as follows:

One hundred milligram of the sampled leaf material was used for RNA extraction. One microgram of RNA was used per RT reaction with Maxima kit (Fermentas, Thermo Fisher Scientific Inc, US). Transcript levels of two reference genes PP2A (SEQ ID No.: 62) (AT1G13320.1) and RNA Helicase (SEQ ID No.: 63) (AT1G58050.1) were measured for normalization of the measured transcript data; since expression of these genes was found to be both stable at the selected developmental stages and not affected by decreased light intensity. ATG5 and ATG7 transcripts were detected using corresponding qPCR primers (Table 1). qPCR reactions were performed in technical triplicates using IQ5 PCR Thermal Cycler (Bio-Rad, Sweden) and DyNAmo Flash SYBR Green qPCR Kit (Finnzymes, Thermo Fisher Scientific Inc, US). qRT-PCR data analysis was performed according to the comparative CT method (Livak and Schmittgen, 2001) with qRT-PCR efficiency correction determined by the slope of standard curves. Fold-differences in transcript levels and mean standard error were calculated as described (Schmittgen and Livak, 2008).

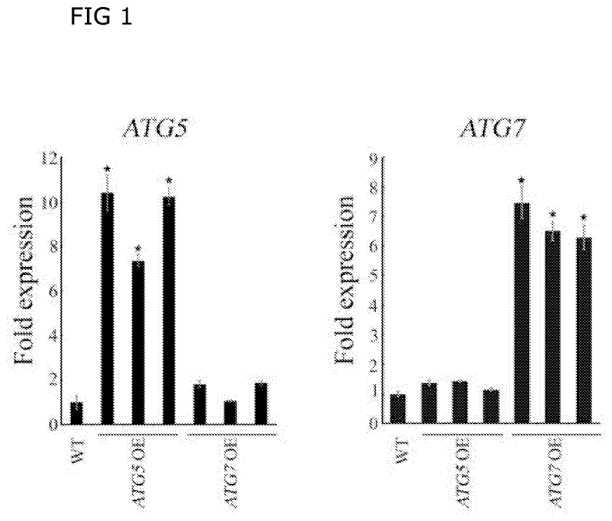

The ATG5 or ATG7 transcript levels in the generated homozygous transgenic lines were from 6.5 to 10.5-fold higher compared to the corresponding transcript levels in wild type A. thaliana Col-0 (WT, Col-0) plants (FIG. 1). Accordingly, the presence of the ATG5 or ATG7 transgenes in the homozygous transgenic lines, each driven by a constitutive promoter, leads to over-expression of these genes, since the total level of ATG5 or ATG7 transcripts is significantly enhanced over WT plants comprising only native genes encoding ATG5 or ATG7.

1.3 Genetically Modified Arabidopsis thaliana Comprising Transgenes Expressing ATG5 or ATG7 are Characterized by Enhanced Autophagic Flux

Autophagic flux in transgenic A. thaliana lines over-expressing ATG5 or ATG7, as compared to wild type A. thaliana (WT, Col-0) plants, was demonstrated by analyzing the lipidation of autophagosomal marker protein Atg8 and the degradation of an autophagic adaptor protein NBR1, which were used as markers of autophagy.

Lipidation of Atg8, which leads to a change in molecule mass, was detected as follows: Seedling samples were homogenized in TNP1 buffer [50 mM 2-amino-2-(hydroxymethyl)-1,3-propanediol (Tris)-HCl (pH 8.0), 150 mM NaCl, 1 mM phenylmethanesulfonyl fluoride, and 10 mM iodoacetamide], and the resulting extract was filtered through cheesecloth and clarified at 2000 g for 5 min. The supernatant was centrifuged at 100 000 g for 1 h, with the membrane pellet then solubilized in TNPI buffer containing 0.5% (v/v) Triton X-100. The solubilized membranes were incubated at 37° C. for 1 h with 250 unit ml⁻¹ of Streptomyces chromofuscus PLD (Enzo Lifesciences, http://www.enzolifesciences.com/) or an equal volume of its companion buffer. Protein samples were subjected to SDS-PAGE in the presence of 6 M urea, and electrophoretically transferred onto polyvinylidene fluoride (PVDF) membranes. Changes in Atg8 molecular mass due to lipidation were detected as change in electrophoretic migration with the aid of Atg8 specific antibodies (as described by Chung et al., 2010).

NBR1 degradation was analyzed as follows: 100 mg of the sampled plant leaf material was mixed with 100 μL of urea extraction buffer (4M Urea, 100 mM DTT, 1% Triton X-100) and incubated on ice for 10 min. Samples were boiled with Laemmli sample buffer for 10 min and centrifuged in a table centrifuge at 13.000 rpm for 15 min. Equal amounts of supernatants were loaded on 12% PAAG and blotted on PVDF membrane. NBR1 was detected with AtNBR1-specific antibodies (Svenning et al., 2011) used at dilution 1:2,000. Reaction was developed using ECL Prime kit (supplied by Amersham, GE Healthcare, Sweden) and detected in LAS-3000 Luminescent Image Analyzer (supplied by Fujifilm, Fuji Photo Film (Europe) Germany). Membranes were stained with Coomassie brilliant blue solution to confirm equal loading. All images were quantified using ImageJ software.

Atg8 lipidation and NBR1 degradation in ATG-overexpressing plants (ATG5 or ATG7-overexpressing plants) was compared to WT plants following cultivation under normal illumination and under darkness/low illumination which stimulates autophagy (FIG. 2). ATG overexpression in the transgenic plants was observed to induce Atg8 lipidation and NBR1 degradation under normal conditions, indicative of a constitutively enhanced basal autophagic flux; which was not observed in WT plants. ATG overexpression also up-regulated autophagic flux when the plants were exposed to autophagy-stimulating conditions, as evidenced by increased Atg8 lipidation in dark grown plants.

In order to exclude the possibility that the observed differences in the abundance of NBR1 protein seen in FIG. 2 were caused by a reduction at the transcriptional level, the levels of NBR1 mRNA in the same leaf samples was quantified by RT-PCR using corresponding qPCR primers (Table 1) according to section Iii. As seen in FIG. 2, NBR1 gene transcript levels are very similar in WT and ATG-overexpressing transgenic plants, indicating that NBR1 transcript levels cannot account for the loss of NBR1 protein.

Accordingly, transgenic A. thaliana lines over-expressing ATG5 or ATG7 are characterized by a constitutively enhanced basal autophagic flux, as evidenced by the response of two independent markers of autophagy in a plant.

### Example 2. Over-Expression of ATG5 or ATG7 in Genetically Modified Arabidopsis thaliana Suppresses Aging and Sustains Flowering

T-DNA knockout lines (atg5 and atg7) of A. thaliana that fail to express either the ATG5 or ATG7 protein are characterized by an earlier onset and a shorter duration of senescence of their rosette leaves as compared to wild type A. thaliana Col-0 (WT, Col-0) plants (Table 2). By contrast, overexpression of either of the ATG genes significantly delayed the onset of leaf senescence (by 4-7 days) without affecting the duration of leaf senescence, as compared to WT, Col-0 plants (Table 2).

TABLE 2
Enhanced autophagy delays onset of leaf senescence

| | | | Duration of |
|---|---|---|---|
| | Onset of rosette | Complete rosette | rosette |
| Genotype | senescence, DAG | senescence, DAG | senescence, days |
| ATG5 trial | | | |
| WT | 36.1 ± 3.74 | 55.6 ± 5.13 | 19.5 ± 5.08 |
| atg5 | 30.2 ± 1.86**** | 44.5 ± 2.13**** | 14.3 ± 2.58**** |
| ATG5 OE | 40.1 ± 3.55** | 58.6 ± 3.52* | 18.5 ± 5.22 ns |
| ATG7 trial | | | |
| WT | 35.1 ± 5.11 | 56.2 ± 5.00 | 21.1 ± 3.50 |
| atg7 | 31.2 ± 4.91** | 47.6 ± 5.39**** | 16.4 ± 4.03**** |
| ATG7 OE | 42.1 ± 5.38** | 62.1 ± 2.53** | 20.0 ± 5.10 ns |

DAG: days after germination (radicle emergence);
OE, overexpression.
atg 5: atg5-1 (Thompson et al. 2005) and
atg7: atg7-2 (Hofius et al. 2009).
All time data are shown as mean ± S.D., with 20 plants per genotype. Each trial was repeated twice, every time with a different overexpression line.
Although there was a variation among replicate trials in the absolute mean values, they all showed the same effects.
*P < 0.05,
**P < 0.01,
***P < 0.001,
****P < 0.0001;
ns, not significant, vs WT in the same trial; Dunnett's test.

The onset of flowering was found to be independent of the level of autophagy, however, the duration of flowering was directly correlated with autophagic flux, so that ATG5- or ATG7-overexpressing transgenic plant lines flowered for approximately 10 days longer than WT plants (Table 3). On average, the lifespan of ATG5- or ATG7-over expressing transgenic plant lines was 10% to 20% longer compared to WT, Col-0 plants (FIG. 3). T-DNA knockout lines (atg5 and atg7) of A. thaliana that fail to express either the ATG5 or ATG7 protein were characterized by a shorter flowering period.

TABLE 3
Enhanced autophagy sustains flowering

| | First flower | Cessation of | Duration of |
|---|---|---|---|
| Genotype | open, DAG | flowering, DAG | flowering, days |
| ATG5 trial | | | |
| WT | 27.5 ± 1.61 | 58.7 ± 9.61 | 31.2 ± 9.89 |
| atg5 | 27.8 ± 1.75 ns | 46.4 ± 1.72**** | 18.6 ± 1.93**** |
| ATG5 OE | 27.4 ± 1.37 ns | 67.6 ± 5.99* | 40.2 ± 7.05* |
| ATG7 trial | | | |
| WT | 30.0 ± 4.36 | 62.5 ± 7.35 | 32.6 ± 6.19 |
| atg7 | 28.5 ± 4.02 ns | 55.3 ± 4.04**** | 26.9 ± 4.77**** |
| ATG7 OE | 32.4 ± 3.28 ns | 74.3 ± 3.25**** | 41.9 ± 3.54**** |

DAG, days after germination (radicle emergence);
OE, overexpression.
atg 5: atg5-1 (Thompson et al. 2005) and
atg7: atg7-2 (Hofius et al. 2009).
All time data are shown as mean ± S.D., with 20 plants per genotype. Each trial was repeated twice, every time with a different overexpression line.
Although there was a variation among replicate trials in the absolute mean values, they all showed the same effects.
*P < 0.05,
**P < 0.01,
***P < 0.001,
****P < 0.0001;
ns, not significant, vs WT in the same trial; Dunnett's test.

Accordingly, transgenic A. thaliana lines over-expressing ATG5 or ATG7, are characterized by a longer life-span, including an extended flowering period.

### Example 3. Over-Expression of ATG5 or ATG7 in Genetically Modified Arabidopsis thaliana Promotes Vegetative Growth and Seed Production

T-DNA knockout lines (atg5 and atg7) of A. thaliana that fail to express either the ATG5 or ATG7 protein were characterized by a 50% reduction, approximately, in both rosette fresh weight (FIGS. 4 A and B) and total weight of mature seeds per plant (FIGS. 4 C and D).

However, overexpression of ATG5 or ATG7 proteins in ATG5- or ATG7-transgenic A. thaliana lines stimulated both vegetative growth and seed yield (FIGS. 4 A, B, C and D). This increase in seed yield was not at the expense of individual seed weight, which was not significantly different from WT, col-0 plants (FIG. 5). The increased seed yield was caused by increased fecundity (seed set) in ATG5- or ATG7-transgenic A. thaliana lines, correlated with the increased duration of flowering in these lines (Table 3). The ATG5- or ATG7-overexpressing plants also developed more and taller inflorescences than respective atg5 and atg7 knockout mutants and WT, Col-A plants (FIG. 4 C).

Accordingly, transgenic A. thaliana lines over-expressing ATG5 or ATG7 proteins are characterized by an increased vigor in terms of longevity, vegetative growth and fecundity.

### Example 4 Over-Expression of ATG5 or ATG7 in Genetically Modified Arabidopsis thaliana Promotes Lipid Accumulation in Seeds

In plants, fatty acids are principal constituents of oil (triacylglycerol) reserves. Arabidopsis accumulates massive amounts of triacylglycerols in seeds, making it a powerful genetic model for identifying genes that regulate/impact oil biosynthesis pathways that have direct application in oil-seed crops.

Total lipid contents, measured as total fatty acids, in genetically modified and wild type A. thaliana plants was determined by converting the acyl groups into methyl esters and quantifying them by GLC. Seed samples (circa 2 mg) were homogenized in methanol/chloroform/0.15 M acetic acid containing 10 mM EDTA (2.5/1.25/0.9 mL) (Bligh and Dyer 1959) using an Ultra Turrax® (IKA). After addition of 1.25 mL of chloroform and 1 mL of water and mixing, the extract was centrifuged and the lipid containing chloroform phase was redrawn. The chloroform phase was evaporated to dryness under nitrogen and the residue re-dissolved in 2 mL methylation solution (2% H₂SO₄ in water-free methanol) and methylated at 90° C. for 1 h. After methylation, 2 mL water and 2 mL hexane were added followed by brief vortexing and centrifugation. GC analysis of fatty acid methyl esters in the hexane phase was performed on a CP-wax 58 (FFAP-CB) column using a Shimadzu gas chromatograph. The identification of fatty acid methyl esters was performed by comparing the retention times with authentic standards (Larodan, Malmö). Quantification of fatty acid methyl esters was done by addition of heptadecanoic acid methyl esters as internal standard prior to methylation.

Overexpression of ATG5 or ATG7 proteins in A. thaliana enhanced the fatty acid content of mature seeds as compared to seeds of WT, Col-A plants (FIG. 6). Since overexpression of ATG5 or ATG7 proteins in transgenic A. thaliana also enhanced seed set (FIG. 4 B), the total yield of seed oil per plant was increased, in the range of 25% to 50% increase compared to WT, Col-0 plants. T-DNA knockout lines (atg5 and atg7) of A. thaliana that fail to express either the ATG5 or ATG7 protein were characterized by a reduced fatty acid content in their seeds and in terms of seed oil yield per plant (FIG. 6).

The increase in seed oil yield in ATG5- or ATG7-transgenic A. thaliana lines as compared to WT, Col-A plants is associated with a preservation of the native fatty acid profile present in seeds of WT, Col-A plants (FIG. 7). By comparison, the fatty acid profile of seeds from T-DNA knockout lines (atg5 and atg7) of A. thaliana, was altered, with an increased content of long-chained species as compared to seed oil of WT, Col-A plants (FIG. 7).

### Example 5 Over-Expression of ATG5 or ATG7 in Genetically Modified Arabidopsis thaliana Promotes Pathogen Resistance

The resistance of genetically modified A. thaliana plants as compared to wild type Col-0 A. thaliana plants to pathogen attack was determined as follows: The necrotrophic fungus, Alternaria brassicicola strain MUCL20297 was cultured on potato dextrose agar plates for 2 weeks at 22° C. Spores were harvested in water and filtered through Miracloth (Calbiochem) to remove hyphae. The spore suspension was adjusted to the final concentration of 5×10⁵ spores mL⁻¹ supplemented with 0.05% Tween 20. A. brassicicola inoculation of three-week-old plants was performed by adding 10 μL drops of spore suspension onto the upper leaf surface as described previously (Thomma et al., 1998). Plants were maintained under saturating humidity for one day prior to pathogen inoculation and two days post inoculation. Leaf samples for fungal quantification were collected 7 days post-inoculation, snap-frozen in liquid nitrogen and stored at −70° C. prior to DNA extraction. Total DNA was extracted from frozen leaf samples using the GeneJET Plant Genomic DNA Purification Kit (Thermo Fisher Scientific) following the manufacturer's protocol. Fungal DNA quantification of three independent biological replicates was carried out by quantitative real-time (qRT)-PCR using the iQ5 qPCR System (Bio-Rad) and primer pairs listed in Table 1.

ATG5- or ATG7-transgenic A. thaliana lines overexpressing ATG5 or ATG7 proteins developed fewer necrotic lesions and suppressed fungal growth as compared to WT, Col-0 plants (FIG. 8). By contrast, T-DNA knockout lines (atg5 and atg7) of A. thaliana developed unrestricted necrotic leaf lesions following inoculation with Alternaria brassicicola thereby greatly facilitating fungal growth (FIG. 8).

Similarly, transgenic A. thaliana lines overexpressing ATG5 or ATG7 proteins showed enhanced resistance to oxidative stress, induced by treating the plants with 0.1 μM methyl viologen (MV) (FIG. 9). Following exposure to MV, the chlorophyll content of ATG5- or ATG7-overexpressing transgenic A. thaliana lines were slightly below those of control (untreated) plants from the same lines; these being much higher than the chlorophyll content of MV-treated WT, Col-0 plants (FIG. 9).

Accordingly, transgenic A. thaliana lines over-expressing ATG5 or ATG7 proteins are characterized by improved resistance to both necrotrophic pathogen infections and oxidative stress.

### Example 6 Identification, Alignment and Structural Annotation of a Plant ATG5 and ATG7 Protein Families

A search, conducted in the phytozome database (http://phytozome.jgi.doe.gov), reveals that ATG5 genes, as well as an ATG7 genes, are present as single copy ortholog genes in most plant genomes. This is strong evidence for the essential role of each of these genes in plants. Members of each ATG protein family and their respective domains were aligned using ClustalX version 2, Larkin et al. 2007. Additional plant ATG 5 and ATG7 orthologs are found by a BLAST search; such as in the Phytozome database. For example, the following steps: select Brassica rapa FPsc v1.3, use BLAST, select Target type: Proteome, remove tick Filter query, then GO, leads to only one gene that is identified as Brara.B02857.1 having 91% amino acid sequence identity to the search query.

6.1 the Plant ATG5 Protein Family

An amino acid sequence alignment of ten members of the family of ATG5 proteins is shown in Table 6; where the following domains are identified (Matsushita et al., 2007): an α1-helix in the N-terminal domain; ubiquitin-like domain, Ub1A; linker 1; an α-helical bundle region (HBR) comprising the catalytic residue Lys149; linker 2; and ubiquitin-like domain, ub1B. The sequence identity between the identified domains in each member of the family of ATG5 proteins and the corresponding domain in A. thaliana ATG5 and their respective locations is given in Table 4 and 6; together with the sequence identities of the full-length sequences of each ATG5 member. The sequence of a native endogenous gene encoding A. thaliana ATG5 is given in the sequence listing [SEQ ID No.: 64].

TABLE 4
Percent amino acid sequence identity between domains of ATG5
family members vs corresponding domains of A. thaliana ATG5

| | | % Amino acid sequence identity | | | | |
|---|---|---|---|---|---|---|
| | | DOMAINS | | | | |
| | | Full | | | | |
| Source | Protein ID | length | Ub1A | Linker | HR | Ub1B |
| Arabidopsis | AT5G17290.1 | 100 | 100 | 100 | 100 | 100 |
| thaliana | | | | | | |
| TAIR10 | | | | | | |
| Brassica | Brara.J01820.1 | 89 | 96 | 86 | 96 | 91 |
| rapa | | | | | | |
| Gossypium | Ciclev10005258m | 69 | 80 | 79 | 80 | 61 |
| raimondii | | | | | | |
| Citrus | Potri.017G139700.1 | 69 | 82 | 79 | 75 | 63 |
| clementina | | | | | | |
| Glycine | Glyma.14G210200.1 | 66 | 79 | 86 | 71 | 53 |
| max | | | | | | |
| Populus | Gorai.007G188600.2 | 64 | 80 | 79 | 78 | 55 |
| trichocarpa | | | | | | |
| Eucalyptus | Eucgr.J01641.1 | 64 | 74 | 86 | 79 | 62 |
| grandis | | | | | | |
| Oryza | TaATG5a_AGW81781.1 | 56 | 70 | 79 | 65 | 49 |
| sativa | | | | | | |
| Triticum | LOC_Os02g02570.1 | 55 | 71 | 64 | 65 | 50 |
| aestivum | | | | | | |
| Zea | GRMZM2G098420_T02 | 53 | 70 | 79 | 62 | 43 |
| mays 6a | | | | | | |

6.2 the Plant ATG7 Protein Family

An amino acid sequence alignment of ten members of the family of ATG7 proteins is shown in Table 7; where the following domains are identified (Noda et al., 2011): N-Terminal Domain (NTD); an Adenylation Domain (AD); and an Extreme C-Terminal Domain (ECTD) domain ending in a C-terminal tail. ATG7 is an E1 enzyme, that in vivo acts as a dimer, and activates the ubiquitin-like proteins ATG8 and ATG12, and transfers them to their cognate E2 enzymes, ATG3 and ATG10 respectively. The sequence identity between the identified domains in each member of the family of ATG7 proteins and the corresponding domain in A. thaliana ATG5 and their respective locations is given in Table 5 and 8; together with the sequence identities of the full-length sequences of each ATG7 member. The sequence of a native endogenous gene encoding A. thaliana ATG7 is given in the sequence listing [SEQ ID No.: 65].

TABLE 5
Percent amino acid sequence identity between domains of ATG7
family members vs corresponding domains of A. thaliana ATG7

| | | % Amino acid Sequence Identity | | | |
|---|---|---|---|---|---|
| | | Domains | | | |
| Source | Protein ID | Full length | NTD | AD | ECTD |
| Arabidopsis thaliana | AT5G45900.1 | 100 | 100 | 100 | 100 |
| TAIR10 | | | | | |
| Brassica rapa | Brara.B02857.1 | 91 | 90 | 94 | 90 |
| Gossypium raimondii | Gorai.XP_012446885.1 | 72 | 72 | 73 | 73 |
| Citrus clementina | Ciclev10014429m | 71 | 71 | 73 | 80 |
| Glycine max | Glyma.12G010000.1 | 71 | 70 | 75 | 68 |
| Populus trichocarpa | Potri.004G055600.1 | 69 | 69 | 71 | 73 |
| Eucalyptus grandis | Eucgr.B01514.1 | 68 | 65 | 73 | 73 |
| Oryza sativa | LOC_OS01g42850.2 | 53 | 49 | 47 | 59 |
| Triticum aestivum | TaATG7_AGW81786.1 | 53 | 48 | 51 | 61 |
| Zea mays 6a | GRMZM2G005304_TO1 | 52 | 49 | 50 | 59 |

TABLE 6
Alignment and structural annotation of the ATG5 proteins

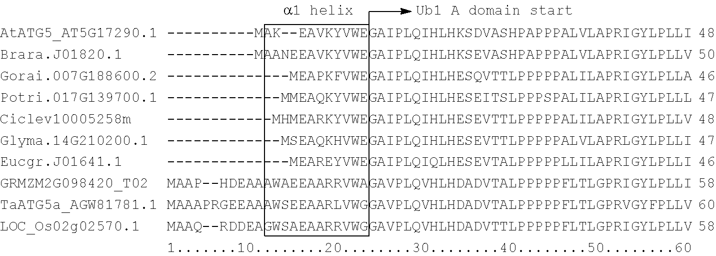

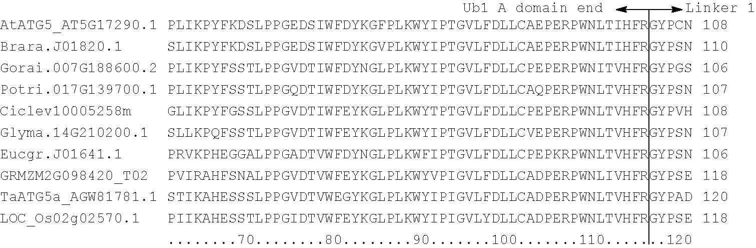

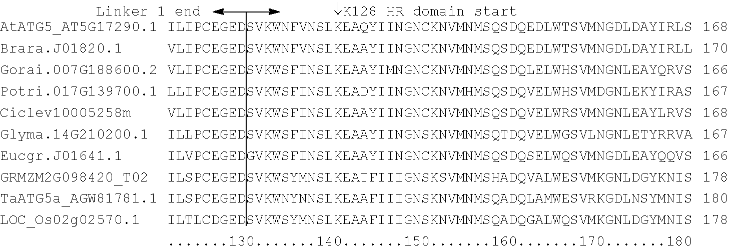

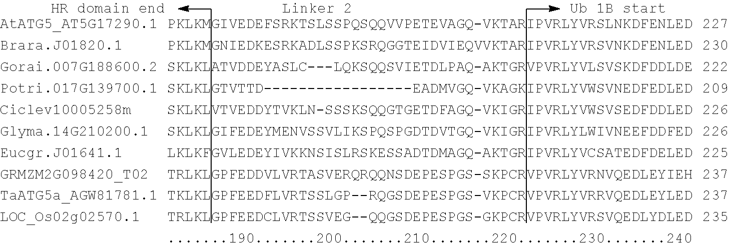

| | | |
|---|---|---|
| AtATG5_AT5G17290.1 | VPEIDTWDDISYLNRPVEFLKEEGKC-FTLRDAIKSLLPEFMGDRAQTSGEERSIDDT-- | 284 |
| Brara.J01820.1 | VPEIDTWDEISYLNRPVEFLREKGKCYFTLRDAIESLLPEYSGDRAQTSGEE-------- | 282 |
| Gorai.007G188600.2 | IPRVDSWEKISYINRPVEIRKE-DKC-FTLHDALKILLPELFLDESLMNVKLGGVDLE-- | 278 |
| Potri.017G139700.1 | APEVDNWDQISYINRPLEIHKQ-GKH-FTLHDALKNLLPEFFGGKSLINDEPCIEEGE-- | 265 |
| Ciclev10005258m | APDIDCWDKISFINRPVEVRTEEGKC-FTLHDAIKTLLPEYFTDKPLFNDESPKLEDE-- | 283 |
| Glyma.14G210200.1 | APQIDNWDKVSYINRPVEIYKEDGKY-FSLNDAVKRILPEFFPENSFVTEGDANINQI-- | 283 |
| Eucgr.J01641.1 | APEIDNWDKISYINRPVEVHKEEGKY-FTLGDAVKALLPAIFTDKSFIDEDVCKTEVEDD | 284 |
| GRMZM2G098420_T02 | AVPVSDWENVSYINRPFETRKAEGRSYITLEHALQTLLPEFFSSKPPGSADGSQHAGAMD | 297 |
| TaATG5a_AGW81781.1 | AIPVSDWESVSYINRPLEIRKEGGRSYIALEHALETLLPEFFSSKPTARAADPEPA-ATT | 296 |
| LOC_Os02g02570.1 | ALPVGDWESISYINRPFEVRREEGRSYITLEHALKTLLPEFFSSKASRIPDDSETAPQAP | 295 |
| | .......250.......260.......270.......280.......290.......300 | |
| AtATG5_AT5G17290.1 | ------EEADGSRE----------------------MGEIKLVRIQGIEMKLEIPFSWVV | 316 |
| Brara.J01820.1 | -------EADGSQET---------------------RGEIKLVRIQGIELKLDIPFSWVV | 314 |
| Gorai.007G188600.2 | -DAVRNSNEDVTSDK-VVEDQGQNACKRPEACRISSSAEIKLIRIQGIEPKLEIPFSWVA | 336 |
| Potri.017G139700.1 | -DVQKVSSEDAGSST-GAEEGKEIFNQPVESC--CNDAEIKLVRIQGIEPKMEIPFSWVV | 321 |
| Ciclev10005258m | -EMN-LSSEDAGSNK-NTEVEEILYEH------VTRNAEIKLVRIQGIEPKLEIPFSWVV | 334 |
| Glyma.14G210200.1 | -EEGESSSDPGSSCN-TLEI-----------------AEIKFVRVQGIEPILDIPFSWVV | 324 |
| Eucgr.J01641.1 | KNAPCVSQEDNQDVSGSAEDRTEKNCEAVEPLFSSNTAELKLLRIHGIEPQLEIPFSWVV | 344 |
| GRMZM2G098420_T02 | AAADSSDATNSS-SRSQEAEQALASPA--EAGSAKRAK-VKLVRVQGVELDMDIPFLWVA | 353 |
| TaATG5a_AGW81781.1 | PGSEPDGSDTSP-GTPRDEKPASAGLQ--ETDVAKKTK-LKLVRVQGIELDMDIPFLWVA | 352 |
| LOC_Os02g02570.1 | DSAPNDDSDVTPRSCEKLESSASSSPQ--EANVANKGKIVKLVRVQGIEVDMDIPFLWVA | 353 |
| | .......310.......320.......330.......340.......350.......360 | |

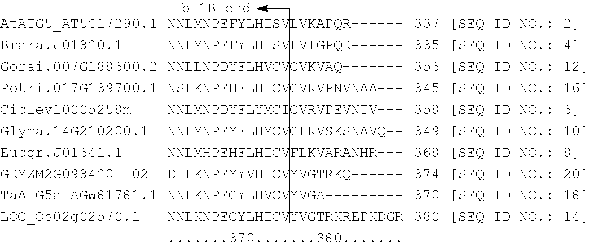

TABLE 7
Position of domains in the amino acid sequence of ATG 5 proteins

| | Ub1A Domain | | Linker | | HR domain | | Ub1B Domain | |
|---|---|---|---|---|---|---|---|---|
| | First | Number | First | Number | First | Number | First | Number |
| | a.a. | of a.a. | a.a. | of a.a. | a.a. | of a.a. | a.a. | of a.a. |
| AT5G17290.1 | 12 | 92 | 104 | 14 | 118 | 55 | 209 | 123 |
| Brara.J01820.2 | 14 | 92 | 106 | 14 | 120 | 55 | 213 | 118 |
| Ciclev10005258m | 12 | 92 | 104 | 14 | 118 | 55 | 209 | 144 |
| Eucgr.J01641.1 | 10 | 92 | 102 | 14 | 116 | 53 | 208 | 153 |
| Glyma.14G210200.1 | 11 | 92 | 103 | 14 | 117 | 55 | 209 | 132 |
| Gorai.007G188600.2 | 10 | 92 | 102 | 14 | 116 | 55 | 205 | 148 |
| LOC_Os02g02570.1 | 22 | 92 | 114 | 14 | 128 | 55 | 218 | 152 |
| Potri.017G139700.1 | 11 | 92 | 103 | 14 | 117 | 55 | 192 | 146 |
| TaATG5a_AGW81781.1 | 9 | 92 | 101 | 14 | 115 | 55 | 190 | 118 |
| GRMZM2G098420_TO2 | 22 | 92 | 114 | 14 | 128 | 55 | 220 | 150 |

TABLE 8
Alignment and structural annotation of the ATG7 proteins

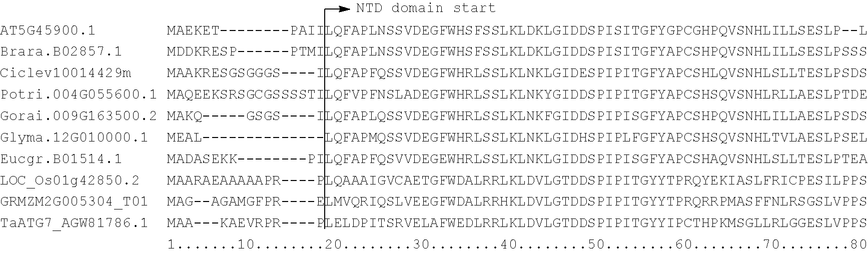

| AT5G45900.1 | DEQSLIASTSHGNRNKCPVPGILYNTNIVESENKLDKQSLLKAEANKIWEDIQSGKALEDPSVLPRELVISFADLKKWSF |
|---|---|
| Brara.B02857.1 | DEQSSTESTSHGNRNKCPVPGTLYNTNIVESETKLDKQSLLKSEANKIWEDIQSGKALEDCALLSRFLVISFADLKKWSF |
| Ciclev10014429m | DEQSSTAEISRGSRNKCIVPGILCNSNTLESFYAIDKQSLLKQEAKKIWEDIHSGKAVEDSTVLSRFLVISFADLKKWSF |
| Potri.004G055600.1 | NDQSSMPAISRGNRNRCPVPGTLYNINTLEAFHALDKKSLLKEEANKIWEDIHNGRAVEDSAVLSRFLLISFADLKKWSF |
| Gorai.009G163500.2 | NDESIMPPVIRGNRNRCYVPGILYNINTMEGFHALYKQALLKAEAMKIWEDIHSGKAVEDCAVLSRVLLISFADLKKWNF |
| Glyma.12G010000.1 | SEASLIPEPSRGNRNRCSVPGILYNTNIVESFHALDKSDLLKKEAAKIWDDILIGKAVEDCSVLSTFLVISFADLKKWIF |
| Eucgr.B01514.1 | SEQACTSSVSLGNRNKCSVPGMIYNTNIFEGYQSLDKQSLLKAETKKIWDDIHSGKALEDSAALSRFLIISFADLKKWNF |
| LOC_Os01g42850.2 | ANSF-------GDRNNCPVPGILLNINNMRGFQNLDRALLLKAEAKKILHDIKSGKVEENPALLLRFLVISFADLKNWKV |
| GRMZM2G005304_T01 | HSSV-------GDRNNCPVPGILINTNNMRGFENLDREQLLKAEAKKILHDIVSGKVEEDPSVLLRFLVISFADLKNWKV |
| TaATG7_AGW81786.1 | ANSS-------GSRNSCPVPGILINTNNMRGLQNLDVEYLLREEAKKILHDIMYGKIEEDPSLLLRFLVISFADLKNWKI |
| | ........90.......100.......110.......120.......130.......140.......150.......160 |
| AT5G45900.1 | RYWFAFPAFVLDPPVSLIELKPASEYFSSEEAESVSAACNDWRDSDLTTDVPFFLVSVSSD-SKASIRHLKDLEACQGDH |
| Brara.B02857.1 | RYWFAFPALVLDPPASLVELKPASEYFTSEEAESVSAACNEWRDSSLTTDVPFFLVSISSDDSKATIRHLKDWEACQGDH |
| Ciclev10014429m | HYWFAFPALVLDPPATVVDLKPASLWFSSQEAESVSAACSDWRNSSLTADVPYFLLTIAPN-SRATIRHLKDWEACEGDG |
| Potri.004G055600.1 | HYWFAFPALVLDPPATLVESKRASEWFTSEEVKSVSVACNDWRNSSLTADVPFFFISIASN-SHATIRHLKDWEACQADN |
| Goral.009G163500.2 | HYWFAFPALALDPPATLVDLRPASQWFTLEEAESVSAACNEWRNSSVTADVPFFLVSIGSD-SHAAVKHLKDLEACQRDG |
| Glyma.12G010000.1 | NYWFAFPALMLDPPATVVNLKPASQWFSAAEAESLSAACNEWRSSKSKTDVPFFLVTIDPN-SRATVRLLKDWEACQSNA |
| Eucgr.B01514.1 | YYWFAFPALVLEPAATLVGIKPASQWLSPEETESLSAACKEWRNSNLTADVPFFLVSIASD-SSVTIRHLKDWEDCKGNG |
| LOC_Os01g42850.2 | YYNVAFPSLIFDSKITLLSLKLASQVLKQEEATSLSNAFTEWRKSSETTVVPFFLINISPD-SSATIRQLKDWKACQGNG |
| GRMZM2G005304_T01 | YYNVAFPSLVFNSRITLLSLQPASKVLTKEEAASMYTSLQKWRTSSETTVIPFFLVSISSD-SSASIRQLKEWKACQGNY |
| TaATG7_AGW81786.1 | YYSVAFPSLVFKSEMTLLSLHSASLVLSQEEAKSLSKSLKEWRSSNETAALPFFFVDISSD-SSIAIRQLKDWKDCQDNG |
| | .......170.......180.......190.......200.......210.......220.......230.......240 |
| AT5G45900.1 | QKLLFGFYDPCHLPSNPGWPLRNYLALIRSRWNLETVWFFCYRESRGFADLNLSLVGQASITLSSGE-SAETVPNSVGWE |
| Brara.B02857.1 | QKLLFGFYDPCHLPSNPGWPLRNYLALIRSKWHLETVWFFCYRESRGFADMSLSLVGQASITLSSD----SSVPNSVGWE |
| Ciclev10014429m | QKLLFGFYDPCHLQNHPGWPLRNFLALILTRWKLKSVLFLCYRENHGFTDLGLSLVGEALITVPQGWGDHQCVPNAVGWE |
| Potri.004G055600.1 | QKVLFGFYDPCHEK-DPGWPLRNFLALISSRWNLKSVHFLCFRESRGFMDMESSLVIEALITAPQGLNDRQLVPNAVGWE |
| Goral.009G163500.2 | QKLLFAFYDPCHLPNNPGWPLRNFLALISARWNLKTVRFLCYRENRGFADLNLSLVGEALITVQQGWREQQCVPNAVGWE |
| Glyma.12G010000.1 | HKILFGFYDPCHLPNNPGWPLRNFLALISARWNLNSVQFFCYRENRGFADMRLSLVGEALITVPQGWKDT--VPSAVGWE |
| Eucgr.B01514.1 | HKLLFGFYDPCHLPHNPGWPLRNYLALIFTKWNIRRIHFLCFRENRGFADMGMSLVGEALISVPEVWKDRNSVPNAVGWE |
| LOC_Os01g42850.2 | QKLLFGFYDHGNR-GFPGWALRNYIAFVSLRWKIEKVHFFCYREKRGRPDIQQSLVGEASFPAPHGWDEPDYVPEAIGWE |
| GRMZM2G005304_T01 | QKLLFGFYDHGCRSDCPGWVLRNYVAFLSIRWKIEKAQIFCYREYKGNPDLEQSLIGEASFPSPYGWDNPEFLPDAIGWE |
| TaATG7_AGW81786.1 | QKFLFGFYDHGCH-QDPGWALRNYIAFLSLRLKIEKIQFLCYREKRSELDLEKSLVGEASFPPPHGWDDSDYVPEAIGWE |
| | .......250.......260.......270.......280.......290.......300.......310.......320 |

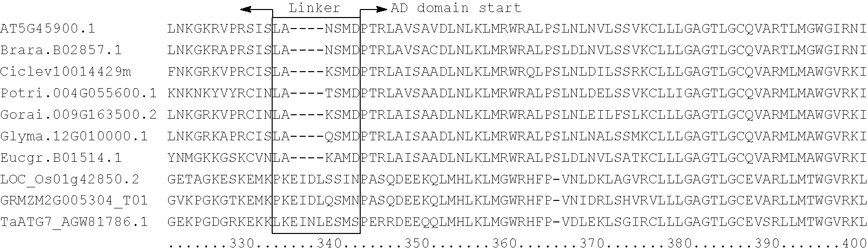

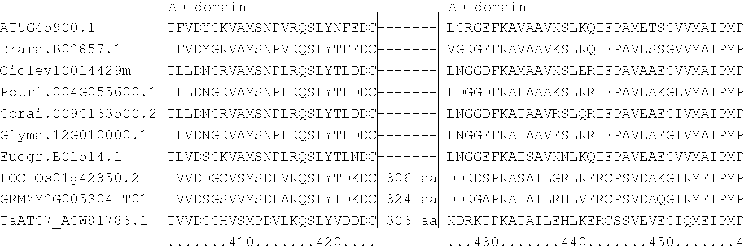

| | |
|---|---|
| | AD domain ↓R¹ |
| AT5G45900.1 | GHPISSQEEDSVLGDCKRLSELIESHDAVFLLTDTRESRWLPSLLCANANKIAINAALGFDSYMVMRHGAGP-------- |
| Brara.B02857.1 | GHPISSQEEESVLGDCKRLRDLIESHDAVFLLTDTRESRWLPSLLCANANKIAINAALGFDSYMVMRHGAGP-------- |
| Ciclev10014429m | GHPVPCQEEDSVLDDCRRLTDLILSHDAIFLLTDTRESRWLPTLLCANTNKITITAALGFDSFLVMRHGPGPFSITHDVK |
| Potri.004G055600.1 | GHPVTNQEEKSVVDDCSRLYDLVDSHDAVFLLTDTRESRWLPTLLCASANKITITAALGFDSFLVMRHGPGPFSSVH--- |
| Gorai.009G163500.2 | GHPVSSQEEKSVLEDCKRLNDLIDSHDVIFLLTDTRESRWLPTLLCANSNKITITAALGFDSFLVMRHGPGPFNSILNLK |
| Glyma.12G010000.1 | GHPVQSQEQDSVLDDCKRLCDLIDAHDSVFLLTDTRESRWLPTLLCANTNKITVTAALGFDSFLVMRHGAGP-------- |
| Eucgr.B01514.1 | GHPVPSQEEESVLNDCRRLHDLISSHDVVFLLTDTRESRWLPTLFSAASSKITITAALGFDSFLVMRHGAGPFSSSNNLK |
| LOC_Os01g42850.2 | GHPVSPNEAVSVLEDCKRLQELVSSHDAVFLLTDTRESRWLPTLLCANENKIAITAALGYDSYLVMRHGAGPGTNCG--- |
| GRMZM2G005304_T01 | GHPVSPGEAAGVLQDCERLKELVLSHDAIFLLTDTRESRWLPTLLCTNENKIAITAALGYDSYLVMRHGAGPGISCE--- |
| TaATG7_AGW81786.1 | GHPVSSKEAAGVLKACERLQELVAAHDAVFLLTDTRESRWLPTLLCANENKIAITAALGYDSYLAMRHGAGPGINSE--- |
| | 60.......470.......480.......490.......500.......510.......520.......530....... |
| | ↓cys |
| AT5G45900.1 | ----TSLSDDMQNLDINKT-NTQRLGCYFCNDVVAPQDSMTDRTLDQQCTVTRPGLAPIAGALAVELLVGVLQHPLGINA |
| Brara.B02857.1 | ----TSLTDDMQNLDMNKA-GRQRLGCYFCNDVVAPQDSMTDRTLDQQCTVTRPGLAPIAGALAVELLVGVLQHPLGIYA |
| Cic1ev10014429m | TEAVNGLSADMDNLCLNNRDGGQRLGCYFCNDVVAPTDSTANRTLDQQCTVTRPGLAPIASALAVELFVGVLHHPKGICA |
| Potri.004G055600.1 | ---ANTSSVDMENLAQTDK-GGKRLGCYFCNDVVAPTDSTANRTLDQQCTVTRPGLAPIASSLAVELFVSILHHPDGMFA |
| Gorai.009G163500.2 | AETENSLAAGMDNLALTNTDGQHRLGCYFCNDVVAPTDSTSNRTLDQQCTVTRPGLAPIASALAVELLVGILHHPNGIFA |
| Glyma.12G010000.1 | ------LSADMP---VNNANGKHRLGCYFCNDVVAPTDSTSNRTLDQQCTVTRPGLAPIASALAVELLVGILHHPQGIFA |
| Eucgr.B01514.1 | TQTLSGPADSVDTASLSHNNGPQRLGCYFCNDVVAPIDSTTNRTLDQQCTVTRPGLAPIASALAVELLVGILHHPHGIHA |
| LOC_Os01g42850.2 | ---SPDVVAAADTLSAEDVLGRQRLGCYFCNDVVAPVDSVSNRTLDQQCTVTRPGLSSITSGCAADLFTRMLHHPDGIHA |
| GRMZM2G005304_T01 | ---ASSVVTAIDKMSTEDALGRQRLGCYFCNDVIAPVDSVSNRTLDQQCTVTRPGLASIASGRAADLFRRMLHHPDGIHA |
| TaATG7_AGW81786.1 | ---GSDMVAAMNKLSAEDVLGRQRLGCYFCNDVIAPVDSVSNRTLDQQCTVTRPGLASIASGHAADLFTRLLNHPDGIHA |
| | 540......550.......560.......570.......580.......590.......600.......610.......6 |

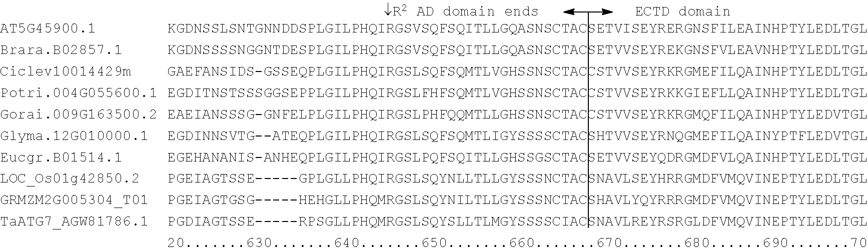

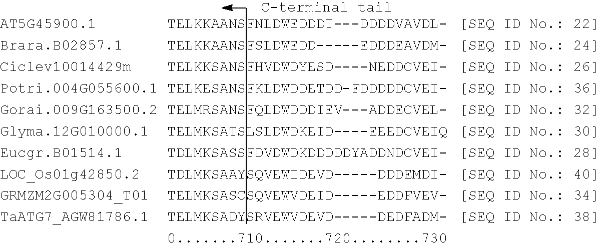

TABLE 9
Position of domains in the amino acid sequence of ATG 7 proteins

| | NTD | | AD | | ECTD | |
|---|---|---|---|---|---|---|
| | domain | | domain | | domain | |
| | Start | Length | Start | Length | Start | Length |
| AT5G45900.1 | 11 | 310 | 327 | 311 | 638 | 41 |
| Brara.B02857.1 | 13 | 310 | 329 | 311 | 640 | 41 |
| Ciclev10014429m | 15 | 313 | 334 | 323 | 657 | 41 |
| Eucgr.B01514.1 | 11 | 313 | 330 | 323 | 653 | 41 |
| Glyma.12G010000.1 | 5 | 311 | 322 | 305 | 627 | 41 |
| Gorai.009G163500.2 | 10 | 313 | 329 | 323 | 652 | 41 |
| Potri.004G055600.1 | 19 | 312 | 337 | 317 | 654 | 41 |
| LOC_Os01g42850.2 | 15 | 305 | 330 | 654 | 984 | 41 |
| TaATG7_AGW81786.1 | 12 | 305 | 327 | 636 | 963 | 41 |
| GRMZM2G005304_T01 | 13 | 306 | 329 | 635 | 964 | 41 |

### Example 7 Overexpression Atg5 and Atg7 in Camelina sativa

The coding region of the two genes ATG5 and ATG7 were cloned down-stream of the napin promoter, GenBank number EU416279.1, and the 35S promoter creating four different constructs. The napin promoter is a seed specific (Ellerstrom et al., 1996) and the 35S promoter is a constitutive promoter expressed in seeds and in the whole plant, respectively.

The four constructs were used to create transgenic Camelina sativa according to standard methods. At least six lines with single insertion has been identified for each construct. Each line equals one motherplant. The relationship is 3:1 of marker gene mCherry and detected as red fluorescent seeds (Shaner et al., 2004). Seeds from three wild type plants were used as reference. The seed weights and total lipid contents are summarized in Table 10 and 11. For each line duplicates of around 20 seeds (18-22) where used

Surprisingly, the napin-ATG7 construct resulted in larger seeds and higher amounts of lipids when compared with wild type seeds.

TABLE 10

| Promoter | Gene | Line No | Weight of seed [mg] | Total lipid % |
|---|---|---|---|---|
| 35S | ATG5 | 2 | 0.81 | 30.6 |
| 35S | ATG5 | 3 | 0.63 | 25.0 |
| 35S | ATG5 | 5 | 0.71 | 27.0 |
| 35S | ATG5 | 6 | 0.72 | 25.5 |
| 35S | ATG5 | 7 | 0.69 | 26.7 |
| 35S | ATG5 | 8 | 0.75 | 26.5 |
| 35S | ATG5 | 10 | 0.81 | 24.5 |
| Napin | ATG5 | 1 | 0.74 | 29.1 |
| Napin | ATG5 | 4 | 0.75 | 28.2 |
| Napin | ATG5 | 7 | 0.72 | 24.4 |
| Napin | ATG5 | 8 | 0.77 | 24.6 |
| Napin | ATG5 | 9 | 0.75 | 24.7 |
| Napin | ATG5 | 10 | 0.76 | 24.9 |
| 35S | ATG7 | 1 | 0.79 | 25.2 |
| 35S | ATG7 | 2 | 0.78 | 25.3 |
| 35S | ATG7 | 4 | 0.74 | 25.5 |
| 35S | ATG7 | 5 | 0.69 | 25.0 |
| 35S | ATG7 | 6 | 0.75 | 25.5 |
| 35S | ATG7 | 9 | 0.80 | 26.3 |
| 35S | ATG7 | 10 | 0.78 | 25.2 |
| Napin | ATG7 | 1 | 0.80 | 25.1 |
| Napin | ATG7 | 2 | 0.85 | 25.0 |
| Napin | ATG7 | 4 | 0.75 | 24.9 |
| Napin | ATG7 | 5 | 0.79 | 26.5 |
| Napin | ATG7 | 6 | 0.81 | 27.5 |
| Napin | ATG7 | 7 | 0.82 | 28.2 |
| Napin | ATG7 | 9 | 0.79 | 26.6 |
| Napin | ATG7 | 10 | 0.78 | 26.4 |
| | WT | 3 | 0.73 | 24.0 |
| | WT | 4 | 0.80 | 27.1 |
| | WT | 5 | 0.78 | 26.5 |

TABLE 11

| Promoter | Gene | Mean Weight of seed [mg] | Total lipid mean % |
|---|---|---|---|
| 35S | ATG5 | 0.73 | 26.5 |
| Napin | ATG5 | 0.75 | 26.0 |
| 35S | ATG7 | 0.76 | 25.4 |
| Napin | ATG7 | 0.80 | 26.3 |
| | WT | 0.77 | 25.9 |

### Example 8 Transcriptome Analysis

To further investigate possible molecular mechanisms underlying the above-described phenotypes of the ATG-overexpressing (OE) plants, we performed complete transcriptome analysis of rosette leaves at two developmental stages. The leaf material was sampled at the budding stage, when no difference in phenotype of wild-type, atg knockout (KO) and ATG-overexpressing plants was detectable. The second sampling was performed ten days after the first flower opened, at the stage when atg knockout plants showed early signs of senescence and differences between wild type and ATG-overexpressing plants became detectable on molecular level (NBR1 degradation, confirmed by Western blot and qPCR).

Expression of each transcript at each time point was firstly normalized to the corresponding values in the wild-type genetic background, after that transcripts were further sorted to select those with similar expression trends in both atg knockout or in both ATG-overexpressing backgrounds. Only transcripts that showed normalized expression trends specific either for knockout or for overexpressing backgrounds were considered for further analysis.

In this study, we observed gene expression trends under normal conditions at the developmental stages corresponding to switch from low to higher autophagic activity. Our results confirm general transcriptional trends in autophagy-deficient plants reported previously and also indicate presence of a complex signaling similar to immune response, induction of pathways managing oxidative stress and elevated response to salicylic acid. We did not observe previously reported upregulation of methionine and ethylene biosynthesis (Masclaux-Daubresse et al., 2014) in either of knockout backgrounds, which might be explained by the differences in sampling stages.

In agreement with the results of phenotypic analysis, number of differentially expressed genes at the first time point was relatively low. Nevertheless, already at this stage we could observe increase in expression of enzymes involved in lipid metabolism in ATG-overexpressing plants and stress- and starvation-related genes in atg knockout plants (FIG. 10 A, B). Surprisingly a trend of decreasing biosynthesis rate was appearing already at such early developmental stage in the knockout background.

At the second time point the number of of differentially expressed genes significantly increased for both atg knockouts and over-expressers and opposite trends became more easily identifiable (FIG. 10 C, D). In general, we could observe groups of genes involved in proteolysis, lipid degradation and salicylic acid signaling, WRKY transcription factors being upregulated in autophagy-deficient plants.

One of the causes of early onset of senescence in ATG-knockout plants was proposed to be their susceptibility to UV light and ROS. This phenomenon has been linked to the decreased production of flavonoids and anthocyanin observed in atg5 and atg9 genetic backgrounds (Masclaux-Daubresse et al., 2014). Interestingly, a large number of genes involved in flavonoid biosynthesis and anthocyanin production are upregulated in ATG-overexpressing plants. Furthermore, at later than 10 DAF stages of development, ATG-overexpressing plants accumulated visibly higher amount of anthocyanin than the wild type (data not shown), thus confirming functionality of transcriptional upregulation of anthocyanin biosynthesis pathway.

Noteworthy, although upregulation of lipid and starch degrading enzymes was detectable in the rosette leaves of knockout plants, genes coding for transport of sugars were significantly downregulated in autophagy-deficient backgrounds and significantly upregulated in ATG-overexpressing plants (FIG. 10 D1 and D2). Transport of sugars from rosette leaves to inflorescence is essential for support of seed onset and development. Thus, higher seed yield of ATG-overexpressing plants might be explained by the combined effect of long lasting rosette and high efficacy of sugar transport towards inflorescence.

Materials and Methods

Arabidopsis plants were grown under 120 uM light, 16 h day, 22° C. Complete rosettes were sampled at the budding stage and 10 days after the first flower opened. Three biological replicates were sampled for each genotype, Table 12. Material was stored at −80° C. prior to RNA extraction. RNA was extracted from the material ground in liquid nitrogen using Spectrum Plant total RNA kit (Sigma), treated with Turbo DNase (Amersham). Quality and concentration of RNA was analysed with NanoDrop and BioAnalyzer, only samples with RIN above 6 were used for further analysis. Expression level of ATG5 and ATG7 was verified for all genotypes by qPCR analysis.

Gene expression assay 8×60K Array XS Arabidopsis and primary normalization and quality control of data were performed at OakLabs, Germany.

Data of satisfying quality was used for further analyses. Common trends in changes of transcriptional profiles for both over-expressers were compared to WT and to both knockout genotypes. Because of ATG5- and ATG7-overexpressing or depleted genotypes were pooled together for the analysis, fold change above 1.5 was considered as significant and p-value lower than 0.1 acceptable.

Venn diagram was built in Venny 2.1.0 to see intersects between common differentially expressed genes. The obtained lists of targets were used for gene ontology using Virtual Plant 1.3 and Classification SuperViewer Tool w/Bootstrap (Provart et al., 2003.)

TABLE 12
Samples used

| | | | Time |
|---|---|---|---|
| group | sample ID | Genotype | point |
| 1 | 1 | Col-0 | 1st |
| 1 | 2 | Col-0 | 1st |
| 1 | 3 | Col-0 | 1st |
| 2 | 7 | atg5-1 KO | 1st |
| 2 | 8 | atg5-1 KO | 1st |
| 2 | 9 | atg5-1 KO | 1st |
| 3 | 13 | ATG5 OE | 1st |
| 3 | 14 | ATG5 OE | 1st |
| 3 | 15 | ATG5 OE | 1st |
| 4 | 16 | ATG7 OE | 1st |
| 4 | 17 | ATG7 OE | 1st |
| 4 | 18 | ATG7 OE | 1st |
| 5 | 22 | Col-0 | 2d |
| 5 | 23 | Col-0 | 2d |
| 5 | 24 | Col-0 | 2d |
| 6 | 25 | atg5-1 KO | 2d |
| 6 | 26 | atg5-1 KO | 2d |
| 6 | 27 | atg5-1 KO | 2d |
| 7 | 31 | ATG5 OE | 2d |
| 7 | 32 | ATG5 OE | 2d |
| 7 | 33 | ATG5 OE | 2d |
| 8 | 34 | ATG7 OE | 2d |
| 8 | 35 | ATG7 OE | 2d |
| 8 | 36 | ATG7 OE | 2d |
| 9 | 40 | atg7-2 KO | 1st |
| 9 | 41 | atg7-2 KO | 1st |
| 9 | 42 | atg7-2 KO | 1st |
| 10 | 43 | atg7-2 KO | 2d |
| 10 | 44 | atg7-2 KO | 2d |
| 10 | 45 | atg7-2 KO | 2d |

### REFERENCES

Bligh, E. G. & Dyer, W. J. (1959) A rapid method of total lipid extraction and purification. Can. J. Biochem. Physiol. 37: 911-917.
Chung, T., Phillips, A. R. & Vierstra, R. D. (2010). ATG8 lipidation and ATG8-mediated autophagy in Arabidopsis require ATG12 expressed from the differentially controlled ATG12A AND ATG12B loci. Plant J. 62:483-493. Clough & Bent, Clough, S. J. & Bent, A. F. (1998) Floral dip: a simplified method for Agrobacterium-mediated transformation of Arabidopsis thaliana. Plant J. 16: 735-743.
Ellerstrom M, Stalberg K, Ezcurra I, Rask L (1996) Functional dissection of a napin gene promoter: identification of promoter elements required for embryo and endosperm-specific transcription. Plant Mol Biol 32: 1019-1027
Hoflus, D. et al. (2009) Autophagic components contribute to hypersensitive cell death in Arabidopsis. Cell 137:773-783.
Klionsky, D. J. et al. (2001) Guidelines for the use and interpretation of assays for monitoring autophagy. Autophagy 8: 445-544.
Larkin et al. 2007, Clustal W and Clustal X version 2.0.Bioinformatics, 23:2947-2948.
Livak, K. J. & Schmittgen, T. D. Analysis of relative gene expression data using real-time quantitative PCR and the 2-ΔΔCT method. Methods 25: 402-408.
Masclaux-Daubresse et al., 2014. Stitching together the Multiple Dimensions of Autophagy Using Metabolomics and Transcriptomics Reveals Impacts on Metabolism, Development, and Plant Responses to the Environment in Arabidopsis. Plant Cell, 26(5): 1857-1877.
Matsushita et al., (2007) Structure of Atg5·Atg16, a Complex Essential for Autophagy. The Journal of Biological Chemistry, 282: 6763-6772.
Murashige T and Skoog F (1962) A revised medium for rapid growth and bio-assays with tobacco tissue cultures. Physiol Plant 15(3): 473-497.
Nakagawa, M et al., (2007) Development of series of gateway binary vectors, pGWBs, for realizing efficient construction of fusion genes for plant transformation. J Biosci. Bioeng. 104: 34-41.
Noda, N N et al., (2011) Structural basis of Atg8 activation by a homodimeric E1, Atg7. Mol Cell. 44(3):462-75
Provart et al., 2003. A Browser-based Functional Classification SuperViewer for Arabidopsis Genomics. Currents in Computational Molecular Biology 2003:271-272.
Salmeron and Vernooij, 1998 Transgenic approaches to microbial disease resistance in crop plants. Current Opinion in Plant Biology, 1:347-352.
Schmittgen, T. D. & Livak, K. J (2008) Analyzing real-time PCR data by the comparative CT method. Nat. Protoc. 3:1101-1108.
Shaner N C, Campbell R E, Steinbach P A, Giepmans B N G, Palmer A E, Tsien R Y (2004) Improved monomeric red, orange and yellow fluorescent proteins derived from Discosoma sp. red fluorescent protein. Nat Biotech 22: 1567-1572
Svenning, S., Lamark, T., Krause, K. & Johansen, T. (2011) Plant NBR1 is a selective autophagy substrate and a functional hybrid of the mammalian autophagic adapters NBR1 and p62/SQSTM1. Autophagy 7: 993-1010.
Thomma, B. P. et al. (1998) Separate jasmonate-dependent and salicylate-dependent defense-response pathways in Arabidopsis are essential for resistance to distinct microbial pathogens. Proc. Natl Acad. Sci. USA 95: 15107-15111.
Thompson, A. R., Doelling, J. H., Suttangkakul, A. & Vierstra, R. D. (2005) Autophagic nutrient recycling in Arabidopsis directed by the ATG8 and ATG12 conjugation pathways. Plant Physiol. 138:2097-2110.

1: A genetically modified plant characterized by enhanced expression of one or more gene encoding an autophagy related ATG5 and/or ATG7 protein as compared to a corresponding wild type plant of the same species; wherein
a. the amino acid sequence of the ATG 5 protein has at least 80% amino acid sequence identity to a sequence selected from the group consisting of: SEQ ID No.: 2, 4, 6, 8, 10, 12, 14, 16, 18 and 20; and
b. the amino acid sequence of the ATG 7 protein has at least 80% amino acid sequence identity to a sequence selected from the group consisting of: SEQ ID No.: 22, 24, 26, 28, 30, 32, 34, 36, 38 and 40; and
wherein the one or more gene comprises a promoter.

2: The genetically modified plant according to claim 1, wherein the one or more gene encoding an autophagy related ATG5 and/or ATG7 protein is a native endogenous gene.

3: The genetically modified plant according to claim 1, wherein the one or more gene encoding an autophagy related ATG5 and/or ATG7 protein is a transgene.

4: The genetically modified plant according to claim 1, wherein the promoter is:
a. a constitutive promoter;
b. a seed-specific promoter, such as a napin promoter; or
c. a modified promoter, where the modification is done by TALENs or CRISPR.

5: The genetically modified plant according to claim 1, wherein the promoter is a constitutive promoter selected from the group consisting of CaMV 35S promoter, opine gene promoter, mannopine synthase promoter, cassava vein mosaic virus (CsVMV) promoter, alfalfa small subunit Rubisco (RbcS) promoter and Populus tomentosa moderate constitutive (PtMCP) promoter.

6: The genetically modified plant according to claim 1, wherein the plant is characterized by one or more phenotypic features selected from the group consisting of:
a. delayed senescence;
b. increased vegetative growth;
c. increased biomass production;
d. increased seed production;
e. increased seed lipid content;
f increased pathogen resistance;
g. increased oxidative stress resistance;
wherein features a) to g) are as compared with a corresponding wild type plant of the same species.

7: The genetically modified plant according to claim 1, wherein the plant is a crop plant or a woody plant.

8: The genetically modified plant according to claim 1, wherein the crop plant is:
a. a monocot plant selected from the group consisting of Avena spp; Oryza spp.; Hordeum spp.; Triticum spp.; Secale spp.; Brachypodium spp.; Zea spp.; or
b. a dicot plant selected from the group consisting of Cucumis spp.; Glycine spp.; Medicago spp.; Mimulus spp; Brassica spp; Camelina spp; and Beta vulgaris.

9: The genetically modified plant according to claim 7, wherein the woody plant is any one of:
a. a hardwood plant selected from the group consisting of acacia, eucalyptus, hornbeam, beech, mahogany, walnut, oak, ash, willow, hickory, birch, chestnut, poplar, alder, maple, sycamore, ginkgo, a palm tree and sweet gum or;
b. a woody plant selected from the group consisting of cypress, Douglas fir, fir, sequoia, hemlock, cedar, juniper, larch, pine, redwood, spruce and yew; or
c. a fruit bearing woody plant selected from the group consisting of apple, plum, pear, banana, orange, kiwi, lemon, cherry, grapevine, papaya, peanut, and fig; or
d. a cotton, bamboo or rubber plant.

10: A method for enhancing the productivity of a plant by genetic modification, comprising the steps of:
a. transforming the plant with at least one transgene encoding an autophagy related ATG5 and/or ATG7 protein; wherein
i. the amino acid sequence of the ATG 5 protein has at least 80% amino acid sequence identity to a sequence selected from the group: SEQ ID No.: 2, 4, 6, 8, 10, 12, 14, 16, 18, and 20; and
ii. the amino acid sequence of the ATG 7 protein has at least 80% amino acid sequence identity to a sequence selected from the group: SEQ ID No.: 22, 24, 26, 28, 30, 32, 34, 36, 38 and 40;
wherein the at least one transgene comprises a promoter,
or
b. introducing a promoter DNA molecule for operable linkage to one or more native endogenous genes encoding an autophagy related ATG5 and/or ATG7 protein in the genome of the plant; wherein
i. the amino acid sequence of the ATG 5 has at least 80% amino acid sequence identity to a sequence selected from the group: SEQ ID No.: 2, 4, 6, 8, 10, 12, 14, 16, 18, and 20; and
ii. the amino acid sequence of the ATG 7 has at least 80% amino acid sequence identity to a sequence selected from the group: SEQ ID No.: 22, 24, 26, 28, 30, 32, 34, 36, 38 and 40;
wherein the promoter DNA molecule is operatively linked to the at least one native endogenous genes.

11: The method for enhancing the productivity of a plant by genetic modification according to claim 10, wherein the promoter is:
a. a constitutive promoter;
b. a seed-specific promoter, such as a napin promoter; or
c. a modified promoter, where the modification is done by TALENs or CRISPR.

12: The method for enhancing the productivity of a plant by genetic modification according to claim 10, wherein the promoter is a constitutive promoter selected from the group consisting of CaMV 35S promoter, opine gene promoter, mannopine synthase promoter, cassava vein mosaic virus (CsVMV) promoter, alfalfa small subunit Rubisco (RbcS) promoter and Populus tomentosa moderate constitutive (PtMCP) promoter.

13: The method for enhancing the productivity of a plant by genetic modification according to claim 10, wherein the plant is characterized by one or more phenotypic features selected from the group consisting of:
a. delayed senescence;
b. increased vegetative growth;
c. increased biomass production;
d. increased seed production;
e. increased seed lipid content;
f increased pathogen resistance;
g. increased oxidative stress resistance;
wherein features a) to g) are as compared with a corresponding wild type plant of the same species.

14: A method for enhancing the productivity of a plant through use of one or more transgenes encoding an autophagy related ATG5 and/or ATG7 protein for enhancing the productivity of a plant comprising cultivating the plant under which the autophagy related ATG5 and/or ATG7 protein is expressed, wherein
a. the amino acid sequence of the ATG 5 protein has at least 80% amino acid sequence identity to a sequence selected from the group: SEQ ID No.: 2, 4, 6, 8, 10, 12, 14, 16, 18, and 20; and
b. the amino acid sequence of the ATG 7 protein has at least 80% amino acid sequence identity to a sequence selected from the group: SEQ ID No.: 22, 24, 26, 28, 30, 32, 34, 36, 38 and 40;
wherein the one or more transgene comprises a promoter.

15: The method for enhancing the productivity of a plant through use of one or more transgenes encoding an autophagy related ATG5 and/or ATG7 protein for enhancing the productivity of a plant according to claim 14, wherein the promoter is:
a. a constitutive promoter;
b. a seed-specific promoter, such as a napin promoter; or
c. a modified promoter, where the modification is done by TALENs or CRISPR.

16: The method for enhancing the productivity of a plant through use of one or more transgenes encoding an autophagy related ATG5 and/or ATG7 protein for enhancing the productivity of a plant according to claim 14, wherein the promoter is a constitutive promoter selected from the group consisting of CaMV 35S promoter, opine gene promoter, mannopine synthase promoter, cassava vein mosaic virus (CsVMV) promoter, alfalfa small subunit Rubisco (RbcS) promoter and Populus tomentosa moderate constitutive (PtMCP) promoter.

17: The method for enhancing the productivity of a plant through use of one or more transgenes encoding an autophagy related ATG5 and/or ATG7 protein for enhancing the productivity of a plant according to claim 13, wherein the plant is characterized by one or more phenotypic features selected from the group consisting of:
a. delayed senescence;
b. increased vegetative growth;
c. increased biomass production;
d. increased seed production;
e. increased seed lipid content;
f increased pathogen resistance;
g. increased oxidative stress resistance;
wherein features a) to f) are as compared with a corresponding wild type plant of the same species.


SEQUENCE LISTING (66 sequences)

SEQ ID NO: 1 - DNA, 1014 bp, Arabidopsis thaliana
  CDS (1)..(1014): AT5G17290.1 (APG5,ATATG5,ATG5) autophagy protein Apg5 family
atggcgaaggaagcggtcaagtatgtatgggaaggagcaattcctctgcagattcatctccacaaatccgacgtcgcttctcaccctgctcctcctcctgctcttgtgttagcaccaagaataggatatttgcctctgttgattcctcttataaagccttatttcaaggattcacttcctcctggtgaagattcaatttggtttgattacaaaggatttcctctaaaatggtatataccaacaggtgttcttttcgatctcctttgtgcagaacccgaaagaccatggaatctcacgatacactttagaggatatccttgcaacatactgataccatgtgaaggagaagattctgtaaaatggaactttgttaattctttgaaagaggcacaatatatcatcaatggaaattgcaagaatgttatgaacatgtctcagagtgatcaagaggatctatggacctctgtcatgaacggtgatcttgatgcctatacaagattatcacccaagcttaaaatgggaacagtcgaagatgagttttcaaggaaaacaagtttgtcatctccacaatctcaacaagttgtgcctgagacggaggtggctggacaagttaagacagcaagaattcctgttcggttgtatgttcgaagtctaaataaagatttcgagaatcttgaagatgtaccggagatcgatacctgggatgacatctcgtaccttaatcgccctgttgagttcctcaaagaagaagggaaatgctttacgttacgtgacgccattaaaagtctcctccctgagtttatgggagacagagcgcaaacgagtggtgaagaaagaagcatagatgatacagaagaagcagatgggtcgagggagatgggtgaaatcaaattggtaaggatacaagggatagaaatgaagctagagataccgttttcgtgggtggtaaataacttgatgaacccagaattctatctccatatctctgtccttgtgaaagctcctcaaaggtga

SEQ ID NO: 2 - protein, 337 aa, Arabidopsis thaliana
MAKEAVKYVWEGAIPLQIHLHKSDVASHPAPPPALVLAPRIGYLPLLIPLIKPYFKDSLPPGEDSIWFDYKGFPLKWYIPTGVLFDLLCAEPERPWNLTIHFRGYPCNILIPCEGEDSVKWNFVNSLKEAQYIINGNCKNVMNMSQSDQEDLWTSVMNGDLDAYTRLSPKLKMGTVEDEFSRKTSLSSPQSQQVVPETEVAGQVKTARIPVRLYVRSLNKDFENLEDVPEIDTWDDISYLNRPVEFLKEEGKCFTLRDAIKSLLPEFMGDRAQTSGEERSIDDTEEADGSREMGEIKLVRIQGIEMKLEIPFSWVVNNLMNPEFYLHISVLVKAPQR

SEQ ID NO: 3 - DNA, 1008 bp, Brassica rapa
  CDS (1)..(1008): Brara.J01820.1
atggcggcgaatgaagaagcggtgaagtatgtatgggaaggagcaataccgctgcaaatccatctccacaaatccgaagtcgcctctcaccctgctcctcctcctgctctcgtgttagcaccaaggatagggtacttgcctctgttagtttctctgataaagccttacttcaaggattcacttcctcctggtgaagactccatctggttcgattacaaaggcgttcctcttaaatggtatataccaacaggtgttctttttgatctcttatgcgctgaacctgaaagaccttggaatctcactatacactttaggggatatcctagcaacgtactcattccatgtgaaggggaagattctgtcaagtggaactttgttaactctttgaaagaggcggcatatatcatcaatggaaactgcaagaatgttatgaatatgtctcagagtgatcaagaggatctgtggacttctgtcatgaacggtgatcttgatgcctacacaaggttattacccaagcttaaaatgggaaatatcgaagataagttttcaagaaaagcagatctgtcatcgccaaaatctcgacaaggtgggactgagatagatgtgacagaacaagttgttaagacagcgaggattcctgtgcggctgtacgttcgaagtgtcaataaagatttcgagaatcttgaagatgtgcctgagatcgatacatgggatgagatttcgtacttgaatcgacctgttgagttcctcagagaaaaagggaaatgctactttactctgcgtgacgccattgaaagtctcctcccggagtacagtggagacagagctcaaacgagtggagaagaagaagcagatgggtcgcaggagacgaggggagaaatcaagctggtaaggatacaagggatagaattgaaactagacataccgttttcatgggtggtaaacaacttgatgaacccagaattctatctccacatctctgttcttgtgataggtcctcaaaggtaa

SEQ ID NO: 4 - protein, 335 aa, Brassica rapa
MAANEEAVKYVWEGAIPLQIHLHKSEVASHPAPPPALVLAPRIGYLPLLVSLIKPYFKDSLPPGEDSIWFDYKGVPLKWYIPTGVLFDLLCAEPERPWNLTIHFRGYPSNVLIPCEGEDSVKWNFVNSLKEAAYIINGNCKNVMNMSQSDQEDLWTSVMNGDLDAYTRLLPKLKMGNIEDKFSRKADLSSPKSRQGGTEIDVTEQVVKTARIPVRLYVRSVNKDFENLEDVPEIDTWDEISYLNRPVEFLREKGKCYFTLRDAIESLLPEYSGDRAQTSGEEEADGSQETRGEIKLVRIQGIELKLDIPFSWVVNNLMNPEFYLHISVLVIGPQR

SEQ ID NO: 5 - DNA, 1077 bp, Citrus clementina
  CDS (1)..(1077): Ciclev10005258m
atgcatatggaagctcgaaaatatgtgtgggaaggagcaattccgcttcagattcatctccatgaatctgaagttaccactctccctcctcctccccccgctttgatattggcgccaagaattggttaccttccgttgttagttggtctcattaaaccctattttggtagctcacttcctcctggagttgacaccatttggtttgaatacaaaggcttgcctctcaaatggtacacacctactggagttctctttgatcttctatgcccggaaccggaaagaccttggaatctcacggttcattttagagggtatccagtgcatgtattgatcccatgtgaaggcgaagattctgtaaagtggagctttattaattcattgaaagaggcagcatatataattaatggaaactgcaagaatgtcatgaacatgtcccaatctgaccaggtggagctttggcgctctgtgatgaatggtaatctggaagcttacctacgggtgtcttctaagcttaaacttgtaacagttgaagatgattacacagtaaaattgaattcgtcgtcaaaatctcaacaaggcactggcgagacagattttgctggacaagtcaagacaggtagaattcctgtacgactgtatgtttggagtgtaagcgaggattttgatgatttagaagatgctcctgatattgactgctgggataaaatctctttcattaaccgacctgttgaggttcgcacagaagaaggtaaatgcttcaccctacatgatgctatcaaaactcttctgccagaatattttaccgacaaacccttgttcaacgatgaatccccaaaactagaagatgaagaaatgaacctctcttctgaagatgcgggcagcaacaaaaatacagaagtggaagaaattttgtatgaacatgtgaccagaaatgctgaaatcaagcttgtgcgcattcaaggaattgaaccaaaattggaaatacctttctcttgggtggtaaacaacttaatgaaccccgactacttcctttatatgtgtatatgcgtcagagttccagaagtgaacaccgtgtga

SEQ ID NO: 6 - protein, 358 aa, Citrus clementina
MHMEARKYVWEGAIPLQIHLHESEVTTLPPPPPALILAPRIGYLPLLVGLIKPYFGSSLPPGVDTIWFEYKGLPLKWYTPTGVLFDLLCPEPERPWNLTVHFRGYPVHVLIPCEGEDSVKWSFINSLKEAAYIINGNCKNVMNMSQSDQVELWRSVMNGNLEAYLRVSSKLKLVTVEDDYTVKLNSSSKSQQGTGETDFAGQVKTGRIPVRLYVWSVSEDFDDLEDAPDIDCWDKISFINRPVEVRTEEGKCFTLHDAIKTLLPEYFTDKPLFNDESPKLEDEEMNLSSEDAGSNKNTEVEEILYEHVTRNAEIKLVRIQGIEPKLEIPFSWVVNNLMNPDYFLYMCICVRVPEVNTV

SEQ ID NO: 7 - DNA, 1107 bp, Eucalyptus grandis
  CDS (1)..(1107): Eucgr.J01641.1 (Egrandis_v1_0.016700m)
atggaggcgcgggagtacgtgtggaagggagcgatcccgctgcagatccaactccacgagtccgaagtcaccgccctccctccgccacctcctctcctcattctggcgccccggatcgggtacctcccgctgctgattccccgcgtcaagccccacttcggcggcgccctcccccccggcgcggacactgtttggttcgactacaacggcctgcccttgaaatggttcattccgacgggagtccttttcgaccttttatgccccgaaccgaagagaccttggaatttgacggtacatttcagaggatatccaagcaatatattggtcccctgcgaaggtgaagatggtgtaaagtggagttttatcaattctctaaaggaggcagcttatattattaatggaaattgcaaaaatgtcatgaatatgtcccaatctgatcaatcagagctttggcaaagtgtaatgaatggtgatctggaagcctaccagcaggtatcgctaaagcttaaatttggtgtacttgaggatgagtacattgtaaagaagaattcaatttcattgagatccaaagaaagcagtgctgacacagatatggctggacaagctaagacaggtagaattccagtcagactgtatgtttgcagtgccactgaagattttgatgagttagaggatgcacctgaaattgataattgggacaagatctcttacataaatcgtcctgttgaggttcacaaagaagaagggaaatacttcacactaggcgatgcagtgaaggctcttttacctgcgatttttaccgacaaatcattcatcgatgaggatgtgtgtaaaacagaagttgaggatgacaagaatgctccttgtgtttctcaagaggacaaccaagatgtgagtggaagtgctgaagataggacagaaaaaaactgtgaagccgttgagcctcttttttcgtcaaatactgctgaattaaagcttttaagaatccacggcattgagccacaattggagatacctttctcctgggttgtgaacaacttaatgcaccctgaacattttctccacatttgtgtgtttcttaaagttgcacgggcaaatcatcgatga

SEQ ID NO: 8 - protein, 368 aa, Eucalyptus grandis
MEAREYVWKGAIPLQIQLHESEVTALPPPPPLLILAPRIGYLPLLIPRVKPHFGGALPPGADTVWFDYNGLPLKWFIPTGVLFDLLCPEPKRPWNLTVHFRGYPSNILVPCEGEDGVKWSFINSLKEAAYIINGNCKNVMNMSQSDQSELWQSVMNGDLEAYQQVSLKLKFGVLEDEYIVKKNSISLRSKESSADTDMAGQAKTGRIPVRLYVCSATEDFDELEDAPEIDNWDKISYINRPVEVHKEEGKYFTLGDAVKALLPAIFTDKSFIDEDVCKTEVEDDKNAPCVSQEDNQDVSGSAEDRTEKNCEAVEPLFSSNTAELKLLRIHGIEPQLEIPFSWVVNNLMHPEHFLHICVFLKVARANHR

SEQ ID NO: 9 - DNA, 1050 bp, Glycine max
  CDS (1)..(1050): Glyma.14G210200.1 (Glyma14g39095)
atgagtgaagcgcagaagcacgtttgggaaggggcaattcctctccagattcacctccacgaatccgaagtcaccactctccctcctcctccacccgctctcgtgttggcacctcggctaggttatttgcctttgctgatttcgctattgaagcctcagttcagcagcacgcttccacctggagtcgacaccatttggtttgagtacaaaggcctccctctcaagtggtatatacctactggagttctttttgatcttttgtgcgtggagccagagaggccgtggaatttaacggtacacttcagaggatatccaagtaatattttacttccctgtgaaggtgaagacagtgtaaagtggagctttattaactcactgaaagaggctgcatatataataaatgggaatagcaaaaatgtgatgaacatgtctcaaactgatcaggtggagctctggggctctgttttaaatggtaatttggaaacttatcgacgggtggcatcgaagcttaagcttggaacttttgaagatgaatatatggaaaatgttagttcagtcttgattaaatctccacaaagtccaggggatactgatgttacgggacaagtgaagactggtaggattcctgttcgtttatatctgtggactgtcaatgaggagtttgatgattttgaagatgctccacaaattgataattgggacaaagtctcatatatcaatcgacctgttgagatttataaggaagatggtaaatactttagcttaaatgatgcagttaaaagaatattgcccgagttttttccggaaaattcctttgtaactgaaggagatgcaaacatcaaccaaattgaagagggagaaagttcatctgatccagggtcatcttgcaacacacttgagattgctgaaatcaaatttgtacgcgttcaaggaatagaaccaattttagacatacctttttcttgggtggttaacaacttgatgaatcccgagtattttcttcatatgtgtgtttgcttaaaagtttcaaagtccaatgctgtgcaatag

SEQ ID NO: 10 - protein, 349 aa, Glycine max
MSEAQKHVWEGAIPLQIHLHESEVTTLPPPPPALVLAPRLGYLPLLISLLKPQFSSTLPPGVDTIWFEYKGLPLKWYIPTGVLFDLLCVEPERPWNLTVHFRGYPSNILLPCEGEDSVKWSFINSLKEAAYIINGNSKNVMNMSQTDQVELWGSVLNGNLETYRRVASKLKLGTFEDEYMENVSSVLIKSPQSPGDTDVTGQVKTGRIPVRLYLWTVNEEFDDFEDAPQIDNWDKVSYINRPVEIYKEDGKYFSLNDAVKRILPEFFPENSFVTEGDANINQIEEGESSSDPGSSCNTLEIAEIKFVRVQGIEPILDIPFSWVVNNLMNPEYFLHMCVCLKVSKSNAVQ

SEQ ID NO: 11 - DNA, 1071 bp, Gossypium raimondii
  CDS (1)..(1071): Gorai.007G188600.2
atggaagcgccaaagtttgtatgggaaggggcaattcctcttcaaatccatctccacgaatcccaagtcaccactcttcctcctcctcctcctgctctgatcttagctccacggatcgggtatctgccgctgttagcgcctttgattaagccctattttagcagcacacttcctcctggtgttgataccatttggttcgattacaatggcctccctctcaaatggtacataccaactggagttcttttcgatcttctgtgccctgagcctgaaaggccttggaacatcactgtccattttagaggatatcccggaagtgtattgataccttgtgaaggtgaagattctgtgaagtggagctttataaactcattgaaagaggcagcatatataatgaatggaaattgcaagaatgttatgaacatgtctcagtctgatcagctggagctttggcattctgtcatgaatggtaatttggaagcctaccagcgggtctcatctaagcttaaacttgcaacggttgatgatgagtatgcaagtttgtgcttgcaaaaatctcaacaatccgtcatagaaacagatttgcctgcacaagcaaagacaggaagagttccagttagattatatgttctaagcgtaagcaaggattttgatgatttggacgagattcctcgagttgatagttgggagaagatctcttacataaatcgacctgttgagatccgcaaagaagataaatgcttcactttacatgatgctttgaaaatccttctgccggaattgtttttagatgaatccttaatgaatgtaaaattaggcggagtagatcttgaggatgcagttagaaattcgaatgaagatgtgaccagtgataaggtagtggaagaccagggacaaaatgcatgtaaacgaccagaggcttgccgcatttccagtagtgccgaaatcaagcttatccgaattcagggtatcgaaccgaagttagagatacccttttcttgggtagcaaacaatttattaaacccggactattttcttcacgtctgtgtttgtgtcaaagttgcacaataa

SEQ ID NO: 12 - protein, 356 aa, Gossypium raimondii
MEAPKFVWEGAIPLQIHLHESQVTTLPPPPPALILAPRIGYLPLLAPLIKPYFSSTLPPGVDTIWFDYNGLPLKWYIPTGVLFDLLCPEPERPWNITVHFRGYPGSVLIPCEGEDSVKWSFINSLKEAAYIMNGNCKNVMNMSQSDQLELWHSVMNGNLEAYQRVSSKLKLATVDDEYASLCLQKSQQSVIETDLPAQAKTGRVPVRLYVLSVSKDFDDLDEIPRVDSWEKISYINRPVEIRKEDKCFTLHDALKILLPELFLDESLMNVKLGGVDLEDAVRNSNEDVTSDKVVEDQGQNACKRPEACRISSSAEIKLIRIQGIEPKLEIPFSWVANNLLNPDYFLHVCVCVKVAQ

SEQ ID NO: 13 - DNA, 1143 bp, Oryza sativa
  CDS (1)..(1143): LOC_Os02g02570.1 autophagy protein 5, putative, expressed
atggcggcgcagcgcgacgacgaggccgggtggtcggcggaggcggcgcggcgggtctggggcggcgccgtgccgctccaggtccacctccacgacgccgacgtcaccaccctccccccacctccgcccttcctgactttggggccaagaatcgggtatcttccactgttggtaccaattataaaggctcattttagcagcacactcccgcctggtattgacactgtttggtttgaatacaaagggctgcccttaaaatggtatatacccattggagttctttacgaccttctatgtgcagatccagaaagaccatggaatctaacagttcattttaggggatatccttctgaaattttaactctctgtgatggcgaagatagtgtaaagtggagctacatgaattccctgaaagaggctgccttcatcatcactggcaacagtaagaatgtgatgaatatgtctcaggctgatcaaggtgctttgtggcaatcagtaatgaaaggtaacttagatgggtatatgaatatctccaccaggcttaagcttggaccattcgaagaggattgcttagtacgaacttcctcagtagagggtcaacaaggttctgatgagcctgaatctcctggatctggcaaaccatgcagggttcctgttcgattatatgtgcgcagtgttcaagaagacctttatgatttagaagatgcactacctgttggtgattgggaaagcatatcctatattaaccgaccattcgaagtacggagggaggaaggtagaagctacatcaccctggaacatgcgttgaaaactttgctacctgagttcttcagctcgaaggcatcacgtatacccgacgattccgaaactgctcctcaggcaccagattcagcccctaatgatgattctgatgtcaccccaagaagttgtgagaagttagagtcgtctgcttcgtcgagcccgcaagaggcgaatgtggccaacaagggtaaaatagtgaagctagtaagggtgcaaggcattgaggtcgacatggacatacctttcctttgggttgccaacaacctgaagaaccctgaatgttacctccatatttgtgtgtatgtcggtacaagaaaacgagagcctaaggatggtagatga

SEQ ID NO: 14 - protein, 380 aa, Oryza sativa
MAAQRDDEAGWSAEAARRVWGGAVPLQVHLHDADVTTLPPPPPFLTLGPRIGYLPLLVPIIKAHFSSTLPPGIDTVWFEYKGLPLKWYIPIGVLYDLLCADPERPWNLTVHFRGYPSEILTLCDGEDSVKWSYMNSLKEAAFIITGNSKNVMNMSQADQGALWQSVMKGNLDGYMNISTRLKLGPFEEDCLVRTSSVEGQQGSDEPESPGSGKPCRVPVRLYVRSVQEDLYDLEDALPVGDWESISYINRPFEVRREEGRSYITLEHALKTLLPEFFSSKASRIPDDSETAPQAPDSAPNDDSDVTPRSCEKLESSASSSPQEANVANKGKIVKLVRVQGIEVDMDIPFLWVANNLKNPECYLHICVYVGTRKREPKDGR

SEQ ID NO: 15 - DNA, 1038 bp, Populus trichocarpa
  CDS (1)..(1038): Potri.017G139700.1 (POPTR_0017s02000, eugene3.00440154)
atgatggaggcacagaaatacgtatgggaaggagcaattccattacaaattcatctccatgaatcagaaataacaagcctcccaccaccatctccagcattaatcttagcacctagaattggttacttacctctgcttcttcctcttattaagccttacttctcttctactctccctcctggacaagatactatttggtttgattacaaaggattgcctctaaaatggtatataccaactggggtgctttttgatcttctatgtgcacaacctgaaaggccttggaatttgacggtacattttagaggttatcctagtaatttgttgattccttgcgaaggtgaagagtctgtaaagtggagctttattaattcactgaaagaggcagattacattatcaatggaaattgcaagaatgttatgcacatgtctcaatctgatcaagtggagctgtggcattctgtcatggatggtaatttggagaaatatatacgtgcatcttcaaagcttaaacttggaacagtcaccactgatgaggcagatatggttgggcaagtgaaggcaggtaaaattccagttagattgtatgtttggagtgttaatgaggattttgaagatctggaagatgcaccagaagttgataactgggatcaaatttcttatataaatcgtcctcttgaaattcacaaacaaggaaaacatttcacccttcacgatgcactgaaaaatctcctgccagaattttttgggggcaaatccttgatcaatgatgaaccatgcatagaagaaggtgaagatgtacagaaggtttcatcagaagatgcaggcagcagtacaggagctgaagaggggaaagaaatctttaaccaacccgtggagtcttgctgcaacgatgctgaaattaaactcgtccgcatccaagggattgaaccaaagatggaaatacctttctcttgggtggtgaacagcctaaagaatcctgagcactttcttcatatctgtgtttgtgtcaaagttccaaatgtaaatgctgcatga

SEQ ID NO: 16 - protein, 345 aa, Populus trichocarpa
MMEAQKYVWEGAIPLQIHLHESEITSLPPPSPALILAPRIGYLPLLLPLIKPYFSSTLPPGQDTIWFDYKGLPLKWYIPTGVLFDLLCAQPERPWNLTVHFRGYPSNLLIPCEGEESVKWSFINSLKEADYIINGNCKNVMHMSQSDQVELWHSVMDGNLEKYIRASSKLKLGTVTTDEADMVGQVKAGKIPVRLYVWSVNEDFEDLEDAPEVDNWDQISYINRPLEIHKQGKHFTLHDALKNLLPEFFGGKSLINDEPCIEEGEDVQKVSSEDAGSSTGAEEGKEIFNQPVESCCNDAEIKLVRIQGIEPKMEIPFSWVVNSLKNPEHFLHICVCVKVPNVNAA

SEQ ID NO: 17 - DNA, 1113 bp, Triticum aestivum
  CDS (1)..(1113): TaATG5a_AGW81781.1
atggcggcggcgccgcggggcgaggaggcggcggcgtggtcggaggaggcggcgcggctggtctggggcggcgccgtgccgctccaggtccacctccacgacgccgacgtcaccgcgctcccaccgcccccgcccttcctgactttagggccaagagttgggtattttccattgttggtgtcgactataaaggctcatttcagcagctcgctcccaccaggtgttgatactgtttggtttgggtataaagggctgccactgaaatggtatatacccattggtgttcttttcgaccttctctgtgcagatccagaaaggccatggaatctaacagttcattttagggggtatcctgcagatatattatcaccatgtgaaggtgaagatagtgtaaagtggaactacaacaattccctgaaagaggctgccttcatcataactggaaacagtaagaatgtgatgaatatgtcccaggctgatcaacttgctatgtgggaatcagtgaggaaaggtgacttgaatagttatatgaatatctcgaccaagcttaagcttggaccgtttgaagaggacttcttagtacggacttcctcgttagggcctcgacaaggttctgatgagcctgaatctcctggatctgtcaaaccatgcagggtacctgttcgattatacgtgcgcagagttcaagaagacctcgagtatttagaagatgcgattcctgtcagtgactgggaaagtgtatcctatataaatcggccactcgagatccggaaggagggaggtagaagctacatcgccttggaacatgccttggagacgttgctaccggagttcttcagctcgaagcccacagctagagctgccgatcctgaacccgcggcgacgacaccaggctcagaacccgacggttcagataccagtccaggcactcctcgtgacgagaaaccagcttcggcgggtctgcaagagacagatgtggccaagaagaccaaactgaagctggtgagagtgcaaggcatcgagctcgacatggacataccgttcctctgggtggccaacaacctgaagaaccccgaatgctacctccatgtctgtgtatacgttggggcatga

SEQ ID NO: 18 - protein, 370 aa, Triticum aestivum
MAAAPRGEEAAAWSEEAARLVWGGAVPLQVHLHDADVTALPPPPPFLTLGPRVGYFPLLVSTIKAHFSSSLPPGVDTVWFGYKGLPLKWYIPIGVLFDLLCADPERPWNLTVHFRGYPADILSPCEGEDSVKWNYNNSLKEAAFIITGNSKNVMNMSQADQLAMWESVRKGDLNSYMNISTKLKLGPFEEDFLVRTSSLGPRQGSDEPESPGSVKPCRVPVRLYVRRVQEDLEYLEDAIPVSDWESVSYINRPLEIRKEGGRSYIALEHALETLLPEFFSSKPTARAADPEPAATTPGSEPDGSDTSPGTPRDEKPASAGLQETDVAKKTKLKLVRVQGIELDMDIPFLWVANNLKNPECYLHVCVYVGA

SEQ ID NO: 19 - DNA, 1125 bp, Zea mays
  CDS (1)..(1125): GRMZM2G098420_T02
atggctgccccgcacgacgaggccgcggcgtgggcggaggaggcggcgcgccgggtctgggccggcgccgtgccgctccaggtccacctccacgacgccgacgtcaccgcgctacccccgccgccgcccttcctgactttgggaccaagaattgggtatttgcccctcttgatacctgttataagggctcatttcagcaatgcactcccacctggtgttgacactgtttggtttgaatataaagggctgcctttgaaatggtatgtaccaattggtgttctttttgatcttctatgtgcagatccagaaagaccatggaatctaatagtccattttaggggatatccctcagaaatcttgtcaccatgcgagggtgaagatagtgtgaagtggagctacatgaattccctgaaagaggccaccttcatcattactgggaacagtaagagtgtgatgaatatgtcgcatgctgatcaagtcgctctgtgggaatctgtaatgaaaggtaacttagatgggtataaaaatatctccaccaggcttaagcttggaccatttgaagatgatgtgttagtacggacagcctccgtggagcggcaacgtcaacaaaattctgatgaacctgaatctcctggatctagcaaaccgtgcagggtccctgttcgattatacgtacgcaatgttcaagaagaccttgagtacatagaacatgcagttcctgtcagtgactgggaaaacgtgtcctacataaatcggccatttgagactcgcaaggctgaaggtagaagctacatcaccctggaacatgcgttgcaaacattgcttccggagttcttcagctcaaagcccccaggcagcgctgacggctcccaacacgcaggcgcaatggacgcagccgcagacagttcagatgccaccaactcttcaagtcgttctcaggaggcagagcaagccctggcaagtccagcagaggcgggttctgccaagagagcgaaagtgaagctggtgagggtgcaaggtgttgagctcgacatggacatcccgttcctgtgggtcgccgaccacctcaagaaccccgaatactacgtccacatctgtgtatacgtcggcactagaaaacaatag

SEQ ID NO: 20 - protein, 374 aa, Zea mays
MAAPHDEAAAWAEEAARRVWAGAVPLQVHLHDADVTALPPPPPFLTLGPRIGYLPLLIPVIRAHFSNALPPGVDTVWFEYKGLPLKWYVPIGVLFDLLCADPERPWNLIVHFRGYPSEILSPCEGEDSVKWSYMNSLKEATFIITGNSKSVMNMSHADQVALWESVMKGNLDGYKNISTRLKLGPFEDDVLVRTASVERQRQQNSDEPESPGSSKPCRVPVRLYVRNVQEDLEYIEHAVPVSDWENVSYINRPFETRKAEGRSYITLEHALQTLLPEFFSSKPPGSADGSQHAGAMDAAADSSDATNSSSRSQEAEQALASPAEAGSAKRAKVKLVRVQGVELDMDIPFLWVADHLKNPEYYVHICVYVGTRKQ

SEQ ID NO: 21 - DNA, 2094 bp, Arabidopsis thaliana
  CDS (1)..(2094): AT5G45900.1 (APG7,ATAPG7,ATATG7,ATG7) ThiF family protein
atggctgagaaagaaactccagcaatcatacttcaattcgctccattgaacagttccgtcgacgaaggtttctggcatagcttctcctctttgaaactcgacaaactcggaatcgatgattctccgatttcaatcaccggtttctatggaccatgtggacatccacaagtctcaaatcatttgactcttttatcagagtcattgcctttagatgaacaatcattgattgctagtactagtcatggaaataggaataagtgtcctgttcctgggattttgtataacaccaataccgttgaaagcttcaataaacttgacaaacaaagtctgctaaaagcagaagcaaataagatttgggaggatattcaatcaggaaaagctcttgaagacccttctgtgttaccaagattcctcgttatctcctttgcagaccttaagaaatggagttttcggtattggtttgcgttccctgcgtttgtacttgatcctcctgtaagtttgattgaattaaagccagcttcagagtattttagttctgaagaggctgaatcagtatccgctgcttgtaatgattggcgcgactcagatttaactactgatgttccattctttttggtttcggtttcttctgattcaaaagctagtatcagacatcttaaagacttggaagcgtgccaaggtgatcatcaaaagttgcttttcggtttctatgacccgtgtcaccttcctagtaatcctggctggccacttcgaaactacttggcactgattcgctcaagatggaacctcgagacagtttggttcttctgctacagagagagtcgtggtttcgctgacttgaacctttctcttgttggtcaagcctcaattacactttcatcaggtgaatcagctgagactgtacctaactcagtgggatgggagcttaacaaagggaaacgtgtaccccgatccattagccttgctaattccatggatccaacaaggttggctgtttctgctgtcgatttaaacttaaagttaatgagatggcgagcattaccgtctcttaacttaaatgtattgtcctctgtcaaatgccttcttcttggagctggtacattgggttgccaagttgctcgtacccttatgggttggggaatccgcaatatcacctttgttgactatggaaaagttgccatgtctaatccagtcaggcaatctctctataattttgaagattgtctaggtcgtggtgagtttaaagctgttgctgctgttaaaagccttaaacagatatttccagcaatggaaactagtggagttgttatggctataccgatgcccggacatccgatttctagccaagaagaagattctgttctcggtgattgtaaacgccttagcgagttgattgaatctcatgatgcggtgttcttgttaaccgatacaagagaaagcaggtggttaccgtctcttctttgtgctaatgccaataagatcgctataaacgcggctttaggttttgacagctacatggtaatgcgccatggtgctggccccacctctctgtctgatgatatgcagaatcttgacataaacaagacaaatacacagagacttggttgttacttctgcaacgacgtggttgcacctcaggattcaatgactgatcgaactctagaccaacaatgcactgttacacgcccgggtctagctcctattgcaggagctctcgccgttgaactcttagtcggagtcctacaacacccacttggtatcaatgcaaaaggcgacaattcgagcttgagtaatacaggaaacaatgatgattcgcctctcgggatattacctcatcagatacgaggctcagtttctcagttctcacagatcacgctgcttggtcaagcttccaacagctgtaccgcttgctctgaaaccgtgatatcggagtatagggagagaggaaacagtttcatacttgaagctattaaccatcctacatacctggaggatcttactggtttaaccgagcttaagaaagctgctaattcattcaacctcgactgggaagacgatgatactgatgatgacgatgtagctgtagatctttaa

SEQ ID NO: 22 - protein, 697 aa, Arabidopsis thaliana
MAEKETPAIILQFAPLNSSVDEGFWHSFSSLKLDKLGIDDSPISITGFYGPCGHPQVSNHLTLLSESLPLDEQSLIASTSHGNRNKCPVPGILYNTNTVESFNKLDKQSLLKAEANKIWEDIQSGKALEDPSVLPRFLVISFADLKKWSFRYWFAFPAFVLDPPVSLIELKPASEYFSSEEAESVSAACNDWRDSDLTTDVPFFLVSVSSDSKASIRHLKDLEACQGDHQKLLFGFYDPCHLPSNPGWPLRNYLALIRSRWNLETVWFFCYRESRGFADLNLSLVGQASITLSSGESAETVPNSVGWELNKGKRVPRSISLANSMDPTRLAVSAVDLNLKLMRWRALPSLNLNVLSSVKCLLLGAGTLGCQVARTLMGWGIRNITFVDYGKVAMSNPVRQSLYNFEDCLGRGEFKAVAAVKSLKQIFPAMETSGVVMAIPMPGHPISSQEEDSVLGDCKRLSELIESHDAVFLLTDTRESRWLPSLLCANANKIAINAALGFDSYMVMRHGAGPTSLSDDMQNLDINKTNTQRLGCYFCNDVVAPQDSMTDRTLDQQCTVTRPGLAPIAGALAVELLVGVLQHPLGINAKGDNSSLSNTGNNDDSPLGILPHQIRGSVSQFSQITLLGQASNSCTACSETVISEYRERGNSFILEAINHPTYLEDLTGLTELKKAANSFNLDWEDDDTDDDDVAVDL

SEQ ID NO: 23 - DNA, 2094 bp, Brassica rapa
  CDS (1)..(2094): Brara.B02857.1
atggacgataagagagaatctccaccgacgatgatacttcaattcgctccattgaacagctccgtcgacgaaggtttctggcacagcttttcctctttgaaactcgataaactcggaatcgacgattctccgatctccatcaccggattctatgcaccgtgttctcatccgcaagtctcaaaccatctgactcttctttcggagtcattgccttcttcttcagatgaacagtcatctaccgagagtactagtcatgggaataggaacaagtgtcctgttccagggactctctacaacaccaacaccgtcgaaagctttactaaactcgacaaacaaagcttgttaaaatcagaagcaaacaagatttgggaagatattcaatcgggaaaggctctcgaggactgtgctttgttatctaggttcctcgttatctcgtttgccgaccttaagaagtggagctttcgttattggtttgcgttccctgcacttgtgcttgatcctcctgcaagcttggttgagttaaagccagcttcggagtattttacttcagaagaagcagaatcagtatctgctgcttgtaatgagtggcgtgactcaagtttaacaactgatgttccattctttttggtttcgatttcttctgatgattcaaaagctaccatcagacatcttaaagactgggaagcttgccaaggtgatcaccaaaagttacttttcggtttctatgacccgtgtcaccttcctagtaaccctggctggccacttcggaattatcttgcacttattcgctcaaaatggcacctcgagactgtttggttcttctgctacagagagagccgcggtttcgctgacatgagcctttctcttgttggtcaagcctccattacactctcatctgattcatctgtacctaactcagtgggatgggagcttaacaaaggaaaacgagccccccgatccattagccttgctaactccatggatccaacaaggttggctgtttctgcttgtgatttaaacttgaagttaatgagatggcgtgcgttaccgtctctagacttaaacgttctgtcctctgtgaagtgccttcttcttggagctggtacattgggttgtcaagttgctcgtactcttatgggttggggaatccgcaacataacctttgtagactatggcaaagtagctatgtctaatcctgtcaggcaatctctgtacacgtttgaagactgtgtaggccgtggcgagttcaaagccgtagctgctgttaaaagccttaaacagatctttccagctgtggaatctagtggagttgttatggctataccgatgcctggacatcccatctctagccaagaagaagagtctgttctcggtgattgcaaacgccttcgtgatttgattgaatctcatgatgcagtgttcttgttgactgatacaagagaaagcaggtggttaccttctcttctttgtgctaatgctaataagattgctataaacgcggctttaggtttcgatagctacatggtaatgcgacatggcgctggccccacctcgttaactgatgatatgcagaatcttgacatgaacaaggcaggaagacagagacttggttgttacttctgcaacgacgttgttgcacctcaagattcaatgactgatcgaacactggaccagcaatgcactgttacacgcccggggttagctcccattgcaggagctctggccgttgaactcttagtcggagttctacaacatccacttggtatatatgcaaaaggagacaactcgagctcgagtaatggaggaaacaccgatgaatcacctctagggatcttacctcatcagattagaggctcagtttctcagttctcacagatcacactgctaggacaagcctccaacagctgcacagcttgttctgaaactgtggtgtcagagtatcgagaaaaaggaaacagtttcgtacttgaagctgttaaccatcctacatacctggaggatcttaccggtttaaccgagcttaagaaagctgctaattcgtttagcctcgattgggaagacgatgaagacgacgatgaagctgtagatatgtaa

SEQ ID NO: 24 - protein, 697 aa, Brassica rapa
MDDKRESPPTMILQFAPLNSSVDEGFWHSFSSLKLDKLGIDDSPISITGFYAPCSHPQVSNHLTLLSESLPSSSDEQSSTESTSHGNRNKCPVPGTLYNTNTVESFTKLDKQSLLKSEANKIWEDIQSGKALEDCALLSRFLVISFADLKKWSFRYWFAFPALVLDPPASLVELKPASEYFTSEEAESVSAACNEWRDSSLTTDVPFFLVSISSDDSKATIRHLKDWEACQGDHQKLLFGFYDPCHLPSNPGWPLRNYLALIRSKWHLETVWFFCYRESRGFADMSLSLVGQASITLSSDSSVPNSVGWELNKGKRAPRSISLANSMDPTRLAVSACDLNLKLMRWRALPSLDLNVLSSVKCLLLGAGTLGCQVARTLMGWGIRNITFVDYGKVAMSNPVRQSLYTFEDCVGRGEFKAVAAVKSLKQIFPAVESSGVVMAIPMPGHPISSQEEESVLGDCKRLRDLIESHDAVFLLTDTRESRWLPSLLCANANKIAINAALGFDSYMVMRHGAGPTSLTDDMQNLDMNKAGRQRLGCYFCNDVVAPQDSMTDRTLDQQCTVTRPGLAPIAGALAVELLVGVLQHPLGIYAKGDNSSSSNGGNTDESPLGILPHQIRGSVSQFSQITLLGQASNSCTACSETVVSEYREKGNSFVLEAVNHPTYLEDLTGLTELKKAANSFSLDWEDDEDDDEAVDM

SEQ ID NO: 25 - DNA, 2148 bp, Citrus clementina
  CDS (1)..(2148): Ciclev10014429m
atggccgctaaacgagagagcgggagcgggggcggatctatactccaattcgcaccgtttcagagctccgtcgacgagggtttctggcacagattgtcttctcttaagctcaacaagctcggcatcgacgagtctcccattcccatcaccgggttctatgctccttgctcacacttgcaagtgtcaaatcatttgtctcttcttacggaatctttaccatctgattccgatgagcagtcatctacggcagaaatcagccgtgggagtcgcaacaaatgcactgtcccaggcaccctctgcaactcaaacacactggaaagtttttatgccattgacaaacaaagcttgctcaaacaagaagcaaagaagatttgggaggacattcactccggtaaagctgtagaggacagtacagtattatcaaggttcctggttatctcgtttgcagacttgaagaaatggagctttcactactggtttgctttccctgcactggtgctggaccctccagcaaccgttgttgatttgaagccagcttcactatggtttagctctcaagaggcagaatccgtgtcagcagcttgtagtgattggcgtaactcaagcttaacagccgatgtgccatattttttgctcactattgctccaaattcacgtgctactattaggcatctgaaggactgggaagcctgcgaaggtgatggtcaaaagttgctatttggtttttatgatccatgtcatcttcaaaaccaccccggttggcctcttcgcaacttcctcgcacttattttaacgagatggaaactcaagtcagttctctttttatgctacagagagaaccatggtttcacagatctgggcttgtcccttgttggtgaagcattgattacagtaccacaaggatggggggatcatcaatgtgttcccaatgcagtgggatgggaatttaacaaagggaggaaagtacctaggtgtattagccttgctaaatctatggatccaactaggttggccatatctgctgcagatttgaatttaaaattaatgagatggcgtcagttgccatctctgaacctagacatcttatcttctcggaagtgtcttctcctaggagcaggtactcttggatgccaggttgctcgcatgcttatggcttggggtgtccggaaaattacattacttgacaatggcagagtggcaatgtctaatccattgaggcagtcgctttatacattggatgactgcctcaatggtggtgattttaaagcaatggcagcagtgaaaagccttgagaggatatttccagccgtggcggcagaaggtgttgtaatggctataccaatgcctgggcatccagtgccttgtcaagaagaagatagtgttcttgatgactgtagacgcctgactgatttgattctttctcatgatgcaattttcttgttaactgatacaagggaaagtcgatggctccccacacttctttgtgccaatactaacaagattacaataactgcagctcttgggtttgacagcttcttagttatgcgccatggacctggtcctttcagcattacccatgatgttaaaactgaagctgtaaatggtttatctgctgatatggataacctttgcctaaataatagagatggagggcaaagattgggctgttacttctgcaatgatgtggttgctccaactgattcaactgccaaccgcactctggaccaacaatgtactgttacacgtccgggacttgctcctattgcttcggctcttgctgttgaactgtttgttggggtcctgcatcatcctaaagggatatgtgctggggctgagtttgctaactccattgatagtggaagcagtgaacaacctcttggcattttacctcaccagattcgaggttccctttctcagttttctcagatgacactcgttggccactcctcaaacagttgcacagcttgttgcagcactgtggtatccgaatatcggaaaagaggaatggagttcatactacaagcaatcaatcatcctacctatctggaggatctgacaggactaacagagttgaagaaatcagccaactcttttcatgtggactgggattatgaatcagacaatgaagatgattgtgttgaaatctga

SEQ ID NO: 26 - protein, 715 aa, Citrus clementina
MAAKRESGSGGGSILQFAPFQSSVDEGFWHRLSSLKLNKLGIDESPIPITGFYAPCSHLQVSNHLSLLTESLPSDSDEQSSTAEISRGSRNKCTVPGTLCNSNTLESFYAIDKQSLLKQEAKKIWEDIHSGKAVEDSTVLSRFLVISFADLKKWSFHYWFAFPALVLDPPATVVDLKPASLWFSSQEAESVSAACSDWRNSSLTADVPYFLLTIAPNSRATIRHLKDWEACEGDGQKLLFGFYDPCHLQNHPGWPLRNFLALILTRWKLKSVLFLCYRENHGFTDLGLSLVGEALITVPQGWGDHQCVPNAVGWEFNKGRKVPRCISLAKSMDPTRLAISAADLNLKLMRWRQLPSLNLDILSSRKCLLLGAGTLGCQVARMLMAWGVRKITLLDNGRVAMSNPLRQSLYTLDDCLNGGDFKAMAAVKSLERIFPAVAAEGVVMAIPMPGHPVPCQEEDSVLDDCRRLTDLILSHDAIFLLTDTRESRWLPTLLCANTNKITITAALGFDSFLVMRHGPGPFSITHDVKTEAVNGLSADMDNLCLNNRDGGQRLGCYFCNDVVAPTDSTANRTLDQQCTVTRPGLAPIASALAVELFVGVLHHPKGICAGAEFANSIDSGSSEQPLGILPHQIRGSLSQFSQMTLVGHSSNSCTACCSTVVSEYRKRGMEFILQAINHPTYLEDLTGLTELKKSANSFHVDWDYESDNEDDCVEI

SEQ ID NO: 27 - DNA, 2148 bp, Eucalyptus grandis
  CDS (1)..(2148): Eucgr.B01514.1
atggctgatgcttcagagaagaagcctatactccagtttgcgccgtttcagagtgttgtggatgagggtttttggcatcggttgtcttctctcaagctcaacaaattaggcattgatgactctcctattcccatttctggtttctatgcaccttgttcacatgctcaggtatctaatcatctttctctcctcaccgagtctttgccaacggaagcaagtgaacaagcatgcacgtcgtcagttagtcttgggaataggaacaagtgttctgtgccaggcatgatttacaacacaaatacttttgagggctaccagtctcttgacaaacagagcctgttaaaggctgaaaccaaaaagatttgggacgatattcattcaggaaaagctctagaggatagtgcagcgctttcaaggtttctcatcatctcatttgcagacctcaaaaagtggaacttttattattggtttgcttttcctgctcttgtccttgaacctgcagcaactttggttggtatcaagccagcttcacagtggcttagcccagaggagacagaatccttgtcagctgcttgtaaggaatggagaaattcaaatttaactgctgacgtgccattcttcttggtaagtattgcttcggattcatctgtgacaattagacatctgaaggactgggaagactgcaaaggcaatggtcacaagttgttatttggtttttatgacccatgccatctgccccacaatcctggttggcctcttcgcaactaccttgcacttatcttcaccaaatggaacatcagaaggattcactttctctgttttcgggagaatcgtggttttgcagatatgggaatgtcccttgttggtgaagccctaatatcagttccagaagtttggaaggatcgtaactctgtgcctaatgctgttgggtgggagtacaacatgggtaaaaaaggatcaaaatgtgtcaaccttgccaaagcgatggatccaacaaggttagccatatctgctgcggatttgaacttgaaattgatgagatggcgtgctttgccctcactggacttaaatgtcttatctgctactaagtgccttctgttgggggctggcacccttggatgccaggttgcgcgcatgcttatggcttggggtgtcaggaaaattaccctagttgatagcgggaaagtggcaatgtcaaatcccctgaggcagtcattatatacactgaatgactgcttgaatggtggtgagtttaaagccatttcagctgtcaaaaatctcaagcagatattcccagctgtggaagcagaaggtgtcgtaatggctatacctatgcctgggcatcctgttcctagccaagaagaagaaagtgtcctgaatgattgtcgacgcctacatgacttgattagttctcatgatgtagttttcctgttgaccgacaccagggaaagtcggtggctacccacgcttttttcagctgcttctagcaagatcaccataactgctgctttaggatttgatagcttcttggttatgaggcatggagcgggtccttttagctcttcaaacaacttgaaaactcaaactctgagtggtccagctgatagtgtggacaccgcatcgctttctcataataatggtccgcagaggctgggctgttacttctgcaatgacgttgttgcaccaattgattcaaccacaaatcgtactcttgaccagcaatgcactgtcacacgtccaggcttagcacctattgcttcagctcttgcggtcgaactgttggttgggattctgcatcatcctcatgggatccatgctgaaggtgagcatgccaatgcaaatataagtgcaaaccatgaacaaccacttggaattttgccacatcagattcgaggttcccttccacagttttctcagatcactctcttgggtcacagctcgggaagttgtaccgcatgttctgaaactgtggtttccgaatatcaggataggggaatggatttcgtacttcaggctattaatcatccgacttatctggaggatctcaccggcctaacggatcttatgaaatccgcaagctcttttgatgtggactgggacaaggatgacgatgacgactacgctgacgacaacgattgcgtggagatatag

SEQ ID NO: 28 - protein, 715 aa, Eucalyptus grandis
MADASEKKPILQFAPFQSVVDEGFWHRLSSLKLNKLGIDDSPIPISGFYAPCSHAQVSNHLSLLTESLPTEASEQACTSSVSLGNRNKCSVPGMIYNTNTFEGYQSLDKQSLLKAETKKIWDDIHSGKALEDSAALSRFLIISFADLKKWNFYYWFAFPALVLEPAATLVGIKPASQWLSPEETESLSAACKEWRNSNLTADVPFFLVSIASDSSVTIRHLKDWEDCKGNGHKLLFGFYDPCHLPHNPGWPLRNYLALIFTKWNIRRIHFLCFRENRGFADMGMSLVGEALISVPEVWKDRNSVPNAVGWEYNMGKKGSKCVNLAKAMDPTRLAISAADLNLKLMRWRALPSLDLNVLSATKCLLLGAGTLGCQVARMLMAWGVRKITLVDSGKVAMSNPLRQSLYTLNDCLNGGEFKAISAVKNLKQIFPAVEAEGVVMAIPMPGHPVPSQEEESVLNDCRRLHDLISSHDVVFLLTDTRESRWLPTLFSAASSKITITAALGFDSFLVMRHGAGPFSSSNNLKTQTLSGPADSVDTASLSHNNGPQRLGCYFCNDVVAPIDSTTNRTLDQQCTVTRPGLAPIASALAVELLVGILHHPHGIHAEGEHANANISANHEQPLGILPHQIRGSLPQFSQITLLGHSSGSCTACSETVVSEYQDRGMDFVLQAINHPTYLEDLTGLTDLMKSASSFDVDWDKDDDDDYADDNDCVEI

SEQ ID NO: 29 - DNA, 2061 bp, Glycine max
  CDS (1)..(2061): Glyma.12G010000.1
atggaggctttgctgcaattcgcaccgatgcagagctccgttgatgagggtttctggcaccgtttgtcttctttgaagctcaacaagcttggcattgaccattctccaattcccttattcggtttttatgcaccgtgctctcactctcaagtatcaaatcatttaactgttttagctgagtctttaccttctgaattgagtgaggcatcattaatccctgaacccagccgcggcaaccggaacaggtgttctgttccagggatactttacaacactaatactgtggaaagcttccatgcacttgataagagtgacctgttgaagaaagaggcagcaaagatatgggatgacattctaactggaaaagctgtggaggactgttcagtactttcaacattccttgttatttcttttgctgacttgaaaaagtggactttcaactactggtttgctttcccggctctgatgcttgatcctcctgccactgtggttaatctaaagccagcttctcagtggttcagcgcagcagaggctgaatcactctctgcagcttgtaacgagtggcgtagctcaaaatcaaaaacagatgtcccgttttttttagtcactatagatccaaactcacgtgctactgttaggcttctgaaggactgggaagcatgtcagagcaatgctcacaagatcctttttggattttatgacccatgtcatctaccaaataatcctggatggcctcttcgcaacttcttagcacttatttctgcgaggtggaatctcaactctgttcaatttttctgctacagagagaaccgtggttttgcagatatgaggttgtcccttgttggtgaagcactgataacagttccacaagggtggaaagacactgtgcctagtgcagttggatgggaacttaataaggggagaaaagctcctaggtgtataagccttgcgcagtccatggatccaaccaggttggccatatctgctgcagatttgaatttaaagctgatgaggtggcgtgctttgccttctttaaacttgaatgccttatcttccatgaagtgtcttctcctcggagctggaacacttggatgccaggttgcgcgcatgcttatggcatggggtgttcgaaaaattactctagttgacaatggaagggtggctatgtctaacccattgaggcagtctctttatactttggatgactgtcttaatggtggggaatttaaagctacggcagcagttgaaagtcttaaacggatatttccagcagtggaagcagaaggcattgttatggctataccaatgcctggacatccagtacaaagccaggaacaggacagtgtgcttgatgattgtaaacgtttgtgtgatttaattgatgctcatgattccgtttttttattgactgatacaagggaaagtcgatggctcccaacacttctctgtgccaatactaacaagattactgttactgctgcactggggtttgatagtttcttggttatgcgccatggagctggtcctttatctgcggatatgccagtaaataatgcaaatggaaaacatagattgggctgttatttctgcaatgatgtcgtggcaccaaccgattcaacatccaaccgcactttggaccagcaatgtactgtgacccgaccagggctagctcctattgcttcagcccttgcagttgaacttttagtagggattttgcatcatcctcaagggatatttgccgaaggtgatattaacaacagtgtcactggagctactgagcaacctcttggtattttgcctcatcagattcgtggttccctttctcaattttctcaaatgacccttataggttactcctcctccagctgcacagcctgtagtcatacagttgtatcagaatatcgaaaccaaggaatggagttcatacttcaagcaattaattatcctaccttcctagaggatgtcactggactgacagagttgatgaaatcggccacctcgttgtcattggattgggacaaggagatagacgaggaggaggactgtgttgaaattcaataa

SEQ ID NO: 30 - protein, 686 aa, Glycine max
MEALLQFAPMQSSVDEGFWHRLSSLKLNKLGIDHSPIPLFGFYAPCSHSQVSNHLTVLAESLPSELSEASLIPEPSRGNRNRCSVPGILYNTNTVESFHALDKSDLLKKEAAKIWDDILTGKAVEDCSVLSTFLVISFADLKKWTFNYWFAFPALMLDPPATVVNLKPASQWFSAAEAESLSAACNEWRSSKSKTDVPFFLVTIDPNSRATVRLLKDWEACQSNAHKILFGFYDPCHLPNNPGWPLRNFLALISARWNLNSVQFFCYRENRGFADMRLSLVGEALITVPQGWKDTVPSAVGWELNKGRKAPRCISLAQSMDPTRLAISAADLNLKLMRWRALPSLNLNALSSMKCLLLGAGTLGCQVARMLMAWGVRKITLVDNGRVAMSNPLRQSLYTLDDCLNGGEFKATAAVESLKRIFPAVEAEGIVMAIPMPGHPVQSQEQDSVLDDCKRLCDLIDAHDSVFLLTDTRESRWLPTLLCANTNKITVTAALGFDSFLVMRHGAGPLSADMPVNNANGKHRLGCYFCNDVVAPTDSTSNRTLDQQCTVTRPGLAPIASALAVELLVGILHHPQGIFAEGDINNSVTGATEQPLGILPHQIRGSLSQFSQMTLIGYSSSSCTACSHTVVSEYRNQGMEFILQAINYPTFLEDVTGLTELMKSATSLSLDWDKEIDEEEDCVEIQ

SEQ ID NO: 31 - DNA, 2478 bp, Gossypium raimondii
  5′UTR (1)..(163)
  CDS (164)..(2299)
  3′UTR (2300)..(2478)
tggcgtggaggactctttcttcaaggtcttctcaacgatattcttacgattccgaaaagaataaattaagaaaaaaaagaaacaaagaagaaagtttgaaaaaaagggaaaaatcttaaatcgcaggcatattgagtggaatagaaaacgggaaaggaaaagcatggcaaaacaaggaagcggatccatacttcaatttgcgccacttcagagttctgttgacgaaggattttggcaccgattatcttctttgaagctcaacaagtttggcatcgacgattctcctattcctatctccggtttctatgcaccgtgctcgcatcctcaagtatcgaatcatttaactcttttagccgaatccctgccttctgactcaaatgatgaatcaacaatgccgcctgttattcggggtaatcgaaataggtgctacgttcctggcattctttacaatactaatacaatggaaggttttcatgctttgtataaacaagccttgctcaaggcagaagcgatgaagatttgggaggatattcatagtggaaaagctgtggaggattgtgcagtattatcaagggtccttcttatctcatttgcagacctcaaaaaatggaactttcattattggtttgctttccctgctcttgcacttgatcctcctgctaccttggtagatttgaggccagcttctcaatggttcactttggaagaggcagagtctgtatcagcagcatgtaatgaatggcggaattcaagtgtgacggctgatgttccattctttttggttagtattggttctgattcacatgctgccgtcaagcatcttaaggatttggaagcttgtcaacgtgatggtcaaaagttgctatttgcattttatgacccatgccatcttccaaacaatcctggctggcctctccgcaacttcttagcacttatttctgccagatggaatctgaaaactgttcgctttttgtgctatagagagaatcgaggttttgctgatttgaatttgtcacttgttggtgaagccttgattacagttcaacaaggatggagagaacagcagtgtgtgcccaatgctgtaggatgggaacttaacaaggggagaaaagtaccaaggtgtattaaccttgctaagtccatggatccaactaggctagccatatctgccgcagacttaaacttgaaactaatgagatggcgtgctttgccatctcttaacttagaaatcttgttttctcttaagtgccttctccttggagcaggtacccttggatgtcaggttgctagaatgcttatggcttggggtgtccgcaaaattacactacttgataatggaagggtggctatgtctaatccgttgagacagtctctatacactttggatgactgccttaatggtggtgattttaaagccacagcagcagtgagaagcttacaacgaatatttcctgcagtggaagctgaaggtattgttatggccattcctatgcctggacatccagtatccagccaagaagagaaaagtgtgcttgaagattgtaaacgactcaatgatttgattgattctcatgatgtaattttcttgttgactgatactagagagagtcgatggctccctactcttctctgtgccaacagtaacaagataaccattactgcagctctaggatttgatagtttcttggttatgcgccatgggcctggtccttttaactccatccttaacttgaaagctgaaacagaaaactctttagctgctggcatggacaaccttgccctaacaaatacagatggacaacatagactgggttgttacttctgtaatgatgtggttgccccaacagattcaacttcaaaccgcacgttggatcagcagtgcactgttacacgtccagggcttgcacctattgcttctgcactggcagtggagcttttagtgggcattttgcatcaccctaatgggatatttgctgaagctgagattgccaactccagtagcggtggaaactttgagcttccgctgggtattttaccccatcagattcgaggctccctcccccactttcaacagatgacccttttgggccactcctctaacagttgcacagcatgttgtagcactgttgtgtcagaatatcgaaagagaggtatgcaattcatactccaggctattaaccatccaacgtatctggaggatgttactggactaacagagttgatgagatcagcgaactcttttcaactggactgggacgatgatattgaggtcgcagatgatgaatgcgtcgaactatgaagaaacaaattgtactttttcaggtcataattatggcaatttattcttttgtaatacatgttaattgtataataacaaacagaaaaaaaaagagtcaaatttatgtccatgctcagtttcccaccttccaatttcaaacattttaacacaagttgatttgggagatactggcaaatttt

SEQ ID NO: 32 - protein, 711 aa, Gossypium raimondii
MAKQGSGSILQFAPLQSSVDEGFWHRLSSLKLNKFGIDDSPIPISGFYAPCSHPQVSNHLTLLAESLPSDSNDESTMPPVIRGNRNRCYVPGILYNTNTMEGFHALYKQALLKAEAMKIWEDIHSGKAVEDCAVLSRVLLISFADLKKWNFHYWFAFPALALDPPATLVDLRPASQWFTLEEAESVSAACNEWRNSSVTADVPFFLVSIGSDSHAAVKHLKDLEACQRDGQKLLFAFYDPCHLPNNPGWPLRNFLALISARWNLKTVRFLCYRENRGFADLNLSLVGEALITVQQGWREQQCVPNAVGWELNKGRKVPRCINLAKSMDPTRLAISAADLNLKLMRWRALPSLNLEILFSLKCLLLGAGTLGCQVARMLMAWGVRKITLLDNGRVAMSNPLRQSLYTLDDCLNGGDFKATAAVRSLQRIFPAVEAEGIVMAIPMPGHPVSSQEEKSVLEDCKRLNDLIDSHDVIFLLTDTRESRWLPTLLCANSNKITITAALGFDSFLVMRHGPGPFNSILNLKAETENSLAAGMDNLALTNTDGQHRLGCYFCNDVVAPTDSTSNRTLDQQCTVTRPGLAPIASALAVELLVGILHHPNGIFAEAEIANSSSGGNFELPLGILPHQIRGSLPHFQQMTLLGHSSNSCTACCSTVVSEYRKRGMQFILQAINHPTYLEDVTGLTELMRSANSFQLDWDDDIEVADDECVEL

SEQ ID NO: 33 - DNA, 3126 bp, Oryza sativa
  CDS (1)..(3126): LOC_Os01g42850
atggcggcgcgggcggaggcggcggcggcggcgccgcggccgctgcaggcggcggcgatcggcgtctgcgcggagacgggcttctgggacgcgctccgccgcctcaagctggacgtcctcggcaccgacgactcccccatccccatcaccggttattatactccacgccaatatgagaaaattgcaagcctctttaggatatgcccagaatcgatactgccaccttctgccaattcctttggcgataggaacaattgtccagtcccagggaccctactaaacactaataacatgagaggattccaaaacttggacagagcacttctactgaaagctgaagcaaagaagatcttgcatgatattaaatctggcaaagtagaagagaatcctgctttgctactaaggttccttgtgatatcatttgctgatctgaagaattggaaagtatattacaatgtcgcattcccctcgttgatttttgactccaagataactttgctcagcttgaaacttgcctcacaagtgcttaagcaagaagaggcaacatccttgtctaatgcatttactgagtggcgtaaatcaagtgaaacaacagttgttccattctttctgatcaatatatccccagattcatctgctactataagacagctgaaggattggaaagcttgccagggcaatggccagaagttgctattcggattttatgatcatggtaatcgaggttttcctggatgggctcttagaaattatattgcattcgtgagcctgcgttggaagattgagaaagttcattttttctgctaccgagaaaaacgaggacgtcctgacatacagcagtcccttgtcggcgaagcatcatttccggcacctcatggctgggatgaacctgattatgtgcctgaagcaattggatgggaaggagaaacggccgggaaggaatcgaaagagatgaaaccgaaagaaattgatctttcttcaattaatccagcaagtcaagatgaagaaaaacagctgatgcacttgaagcttatgggatggcgccacttccctgtgaatcttgacaagttagctggggtccgttgtcttttgttgggtgctggaactcttggatgtgaagtcgcacgcctacttatgacctggggtgttcggaaactaacagtggttgatgatggttgcgtttccatgtctgatctagtcaaacaatccctctacacggataaggattgtggtgtcccaagagtgactgcaatagtcccacatctaaaagaaagatgttctgcggtggaagttgaaggcatccaaatgggaataccaaagcttgagtataatatttctgccagcaaaatatcaagtattactgatgattgcaagcgtctccaaacattagttgattccaatgatgtggtcttcttgttgaatgaaacatgggagggaatgtggcttccaactcttttatgtgcagacaaaaacaagattgccattactgtgttgttaggatatgacaattaccttgtcatgcgacatggtgctggtccaggaacaaaaagtggaggtatggatgaagggattgctcagatagagaatttgtccacacaagacgctctcggtcggcaaagattgggctgctgtttctgcagtgatactacttcccttgttaattcagatcataatggagcactggatcagcagtcggcagtaatattacctgggctgacctcagttgcatctggcaaagcagtggagctctttgctaggatgttacatcatccagatgaaattcatgctccaggagatattgctggtacagacactgagcatcagcttggtctattgccacatcagatgcaaggatcgctctcaaagtgtgtcttgtcaactgtgctgtgcaattcctcaagcaactgtattgcatgttccaatgctgttttatcagaatatagaagaagaggatttgattttgtaacgcaggccatcacctgtccaacatatttgaaagaccttacaggcatatctgatttgaaaaaaccatttgcttctaaaatttcagccagcattcctgtttctaaaacttcggccagcattcctgtaaacctagagaagctgtccagtgcccgatgtctacttttaggtgctggaacacttggatgtgatgttgctcgtattcttatggactgtggtgtgcggaaactaacagtagttgacagtggtcgagttgttgtgtcgaatttggcaagacagtcactctacacatctgatgatcgtgacagcccaaaagcatcagcaatactagggcgtttgaaagagagatgtccttcagtggatgcaaaaggaattaaaatggaaataccaatgccaggccatcctgtatcacctaatgaagcagtcagtgtgcttgaagattgcaaacgcctccaggagttggtatcttctcatgatgctgtcttcttgctgaccgacacaagggaaagtaggtggcttccaacacttctctgcgcaaatgaaaacaagattgctattactgcagcattaggatatgacagttaccttgtcatgcgacatggggctggtccaggcacgaattgtggaagtccagatgtggttgctgctgctgatacactgtctgcagaagatgttcttggtcgtcagagactaggatgttatttctgcaatgacgtcgttgctcctgttgattccgtgtccaaccgaacactggatcaacaatgtacagtaacacggcctggattgtcctccattacatctggctgtgcggcagacctcttcacaagaatgttacatcatccagatgggatccatgctccaggagagattgctggcacaagcagtgagggtccacttggtctattgccgcatcagatacgaggatcactctcgcagtacaacttactaaccctattgggctattcctcgagcaattgtaccgcatgttccaatgcagtgttgtctgaataccataggagagggatggattttgtgatgcaggtgatcaacgaaccaacctatctggaagacctcaccggccttactgacttgatgaaatcagcggcctattctcaggtcgagtggattgatgaagttgacgatgacgacgagatggatatatga

SEQ ID NO: 34 - protein, 1041 aa, Oryza sativa
MAARAEAAAAAPRPLQAAAIGVCAETGFWDALRRLKLDVLGTDDSPIPITGYYTPRQYEKIASLFRICPESILPPSANSFGDRNNCPVPGTLLNTNNMRGFQNLDRALLLKAEAKKILHDIKSGKVEENPALLLRFLVISFADLKNWKVYYNVAFPSLIFDSKITLLSLKLASQVLKQEEATSLSNAFTEWRKSSETTVVPFFLINISPDSSATIRQLKDWKACQGNGQKLLFGFYDHGNRGFPGWALRNYIAFVSLRWKIEKVHFFCYREKRGRPDIQQSLVGEASFPAPHGWDEPDYVPEAIGWEGETAGKESKEMKPKEIDLSSINPASQDEEKQLMHLKLMGWRHFPVNLDKLAGVRCLLLGAGTLGCEVARLLMTWGVRKLTVVDDGCVSMSDLVKQSLYTDKDCGVPRVTAIVPHLKERCSAVEVEGIQMGIPKLEYNISASKISSITDDCKRLQTLVDSNDVVFLLNETWEGMWLPTLLCADKNKIAITVLLGYDNYLVMRHGAGPGTKSGGMDEGIAQIENLSTQDALGRQRLGCCFCSDTTSLVNSDHNGALDQQSAVILPGLTSVASGKAVELFARMLHHPDEIHAPGDIAGTDTEHQLGLLPHQMQGSLSKCVLSTVLCNSSSNCIACSNAVLSEYRRRGFDFVTQAITCPTYLKDLTGISDLKKPFASKISASIPVSKTSASIPVNLEKLSSARCLLLGAGTLGCDVARILMDCGVRKLTVVDSGRVVVSNLARQSLYTSDDRDSPKASAILGRLKERCPSVDAKGIKMEIPMPGHPVSPNEAVSVLEDCKRLQELVSSHDAVFLLTDTRESRWLPTLLCANENKIAITAALGYDSYLVMRHGAGPGTNCGSPDVVAAADTLSAEDVLGRQRLGCYFCNDVVAPVDSVSNRTLDQQCTVTRPGLSSITSGCAADLFTRMLHHPDGIHAPGEIAGTSSEGPLGLLPHQIRGSLSQYNLLTLLGYSSSNCTACSNAVLSEYHRRGMDFVMQVINEPTYLEDLTGLTDLMKSAAYSQVEWIDEVDDDDEMDI

SEQ ID NO: 35 - DNA, 2148 bp, Populus trichocarpa
  CDS (1)..(2148): Potri.004G055600.1 (POPTR_0004s05470, gw1.IV.2775.1,Pt-APG7.1) similar to Component of autophagy conjugation pathway. Required for proper senescence.; similar to autophagy 7 (APG7); similar to autophagy 7 (Arabidopsis thaliana) GI19912147; [ ortholog of
atggctcaagaggaaaagagtaggagtggatgtggatcttcatcttctactatcctccaattcgtaccctttaatagcttagctgacgaaggtttctggcatagattgtcttctttgaagctcaacaaatatggcatcgatgattctcctatacccatcaccgggttttatgcgccttgctcacattctcaagtatccaatcacctgagacttctcgctgaatctttgccgactgatgaaaatgaccagtcttcaatgccagcaattagtcgcggtaacaggaacagatgccctgtacccgggactctttacaatacaaatacattggaggccttccatgccctggataaaaagagcttgctaaaggaggaagcaaacaagatttgggaagacattcataatgggagagcagtggaggacagtgcggtgctttcaaggttccttcttatatccttcgcagacctaaaaaaatggagctttcattactggtttgctttccctgccttggtgcttgatcctcctgcgacattggttgaatcaaagcgtgcttcagaatggttcacctcagaagaggtaaaatccgtatctgtggcttgtaatgactggcgtaactcaagcttaaccgcagatgttccattcttttttatcagtattgcttccaattcccatgctactatcaggcatttaaaggattgggaagcctgccaggctgataatcaaaaggtgctatttggtttttatgacccatgtcatgaaaaagatcctggttggcctcttcgcaacttcctagcactgatttcttcaagatggaatctgaagagtgtccactttttatgcttcagagagagtcgtggttttatggatatggagtcttcccttgtcattgaagccttaataacagctccacaaggattgaatgatcgtcaacttgtacctaatgcagttggatgggaaaaaaacaaaaataaatacgtatacagatgtattaaccttgctacatccatggacccaactaggttggccgtatctgctgcagatttgaatttaaaactaatgagatggcgtgccttgccatcccttaacttagatgagttatcttctgtgaagtgtcttctcataggagcaggcacgcttggatgccaggttgctcgcatgcttatggcttggggtgtccgaaaaattacactacttgacaatggaagagtggctatgtctaatccattgaggcagtccctgtacacattggatgactgcctcgatggaggtgattttaaagccttggcagccgctaaaagcttgaagcgtatttttccagctgtggaagcaaagggtgaagtgatggctataccaatgcctggtcatccagtgactaaccaagaagagaagagtgtggttgatgattgtagccgtctgtatgatctggttgattcccatgatgcagttttcttgttgactgatacaagagagagccgatggctcccaacccttctatgtgcaagtgctaacaagattactataactgcagctcttgggtttgatagcttcttggtaatgcggcatggccctggtccttttagctctgttcatgctaacacttcatctgttgatatggaaaaccttgctcaaactgacaaaggagggaagagactgggctgttacttctgcaatgatgtggttgcacctacagattcaactgccaatcgaactttagaccaacagtgcactgttacacgtccagggcttgctcctattgcttcatctcttgctgtagagctttttgtcagcatcttgcatcatcctgatgggatgtttgctgaaggtgatatcacaaactccactagtagcagtggtggcagtgagccacctcttggtatattgccccaccagattcggggttctctctttcatttttcgcaaatgacccttgtaggccactcctctaatagttgcactgcttgttgcagcactgttgtatcagaatatcggaaaaaaggaatcgaattcctgcttcaagcaattaatcatcctacttatttggaggatctcaccggactcacagaattgaaggagtcggctaactcatttaaattggactgggatgatgagacagatgacttcgatgatgatgatgattgtgttgaaatataa

SEQ ID NO: 36 - protein, 715 aa, Populus trichocarpa
MAQEEKSRSGCGSSSSTILQFVPFNSLADEGFWHRLSSLKLNKYGIDDSPIPITGFYAPCSHSQVSNHLRLLAESLPTDENDQSSMPAISRGNRNRCPVPGTLYNTNTLEAFHALDKKSLLKEEANKIWEDIHNGRAVEDSAVLSRFLLISFADLKKWSFHYWFAFPALVLDPPATLVESKRASEWFTSEEVKSVSVACNDWRNSSLTADVPFFFISIASNSHATIRHLKDWEACQADNQKVLFGFYDPCHEKDPGWPLRNFLALISSRWNLKSVHFLCFRESRGFMDMESSLVIEALITAPQGLNDRQLVPNAVGWEKNKNKYVYRCINLATSMDPTRLAVSAADLNLKLMRWRALPSLNLDELSSVKCLLIGAGTLGCQVARMLMAWGVRKITLLDNGRVAMSNPLRQSLYTLDDCLDGGDFKALAAAKSLKRIFPAVEAKGEVMAIPMPGHPVTNQEEKSVVDDCSRLYDLVDSHDAVFLLTDTRESRWLPTLLCASANKITITAALGFDSFLVMRHGPGPFSSVHANTSSVDMENLAQTDKGGKRLGCYFCNDVVAPTDSTANRTLDQQCTVTRPGLAPIASSLAVELFVSILHHPDGMFAEGDITNSTSSSGGSEPPLGILPHQIRGSLFHFSQMTLVGHSSNSCTACCSTVVSEYRKKGIEFLLQAINHPTYLEDLTGLTELKESANSFKLDWDDETDDFDDDDDCVEI

SEQ ID NO: 37 - DNA, 3063 bp, Triticum aestivum
  CDS (1)..(3063): TaATG7_AGW81786.1
atggcggcgaaggcggaggtgcggccgcggccgctggagttagaccccatcacgagccgcgtcgagctggcgttctgggaggacctccgccgactcaagctcgacgtcctcggcaccgacgactcccccatccccatcaccggttactatattccatgcacacatccaaaaatgtcgggcctcttaaggctgggcggagaatcattagtgccaccatctgccaactcctctggctctaggaactcttgtccagtcccagggaccctcataaacacgaataacatgcgagggttacaaaacctggatgtggagtatctactgagagaagaagcaaagaagatcttgcacgacattatgtatggtaaaatagaagaggatccttctctgctactgaggttccttgtgatatcattcgcggatctgaagaattggaagatttattacagtgttgcattcccctcattggttttcaagtctgaaatgactctgctcagcctgcatagtgcctcactggtgcttagtcaagaggaggcaaaatcattgtctaagtcactgaaagagtggcgcagttcaaatgaaacagcagctcttccatttttcttcgtcgatatatcctcggattcttctattgctataaggcaactcaaggattggaaagattgccaggacaatggccaaaagttcctatttggattttatgatcatggttgtcatcaagatcctggctgggctcttagaaactacattgcattccttagtctgcggttgaaaattgagaagattcagttcttatgctaccgagaaaaacgatcggagcttgatctagagaaatcccttgttggcgaagcatcatttccaccacctcatggctgggatgactctgattatgtgcctgaagctattggatgggaaggagaaaagcctggggatggaaggaaagaaaagaaactgaaagaaatcaatcttgaatcaatgagcccagaaaggcgggatgaagaacaacaactgatgcacttgaagcttatgggatggcggcacttccctgttgatttggagaagttatctggcattcgatgtcttctgttgggtgctggaactcttggatgtgaagtttctcgcttactcatgacctggggtgtacggaaactaacagtggttgacggtggtcatgtttctatgcctgatgtactcaaacaatcactctacgtagatgacgattgtggtgtcccaagagccaccgcaatagttccacatctaaaagagagatgtcctgcagtggacgttgaagccatccagatggaaataccagcgcctgggaatccggtttctccaagtgtgcttgatgattgtgagcgtcttcaaacattagtagcttctagtgatgtggtcttcttgttgactgacacatgggagagcagatggtttccaactcttttgtgtgcaaacgaaaacaagatggccatcacggcagcattaggacatgacagttaccttgtcatgcgacatggtgctggtccaggaacaagaagaggaaatatggatgacgtgattgctcagatacagaatttatccaccgaagatgctctcggtcgtcaaagattggggtgctgtttctgcaacgatacgacttcccttttcaactcagtctctgatgaaacagtagcactacctgggttgacctccattgtatccggcaaagcggtggagctctttgcgaggatgttacatcatccagaggggatacatgctccaggagatatggctggtatggatactgaacatcagcttggtttattgccgcaccagatgcgaggatcactcccgcggtgtgatttatcaactgtgataagcaattcctcaaacaattgtactgcatgttctaatattgtactgtctgaatatagaagagaagggttgaattttgtcatgcaagccattaaccaaccaacatatttaaaggaccttacaggcatttctaatttgataaagtcggacacttgtttgaaattgccagccagcttccctgtaaattctgggaagttgtctagtgctcgatgtctacttttaggtgctggaactcttgggtgcgatgttgctcgtattcttatggattatggtgttcggaatctcacaattgttgacagtggttgtgttgttgtgtcaaatttggcaagacaatcactttacacaactaaagaccgcaagaccccaaaagcgacagcgatactggagcatctaaaagagagatgttcgtcagtggaggttgaaggaattcaaatggaaataccgatgccagggcaccctgtatcatctaaagaagctgctggtgtgcttaaagcttgcgagcgtctccaggagttagtagctgcccatgatgctgtcttcttgctgactgacacaagggaaagtaggtggctgccaacgcttctctgtgcgaatgaaaacaagattgctattaccgcagccctaggatatgatagctaccttgccatgcgacatggggctggtccaggaataaattctgaaggttcagatatggttgctgccatgaacaagctgtctgcagaagatgttcttgggcgtcaaagactggggtgctatttctgcaatgacgttatcgctcctgttgattcggtctccaaccgaacgctggaccaacagtgtacagtaacacgacctggactggcctccattgcatctggccatgctgcagacctcttcacaagactgttaaatcatccagatgggatacatgctccaggagatatcgctggcacaagcagtgagcgtccgtctggtctattgccacaccagatgcgaggatcactatcgcagtacagcttattaactctcatgggctattcctcgagcagctgtatcgcatgttccaatgcggtattgcgtgaatacaggagtagaggattggattttgtgatgcaggtgatcaacgaaccaacctacctagaagacctgacgggcctgacggagttgatgaaatcggcggattattctcgagtcgagtgggtcgatgaagttgacgacgaggactttgccgacatgtga

SEQ ID NO: 38 - protein, 1020 aa, Triticum aestivum
MAAKAEVRPRPLELDPITSRVELAFWEDLRRLKLDVLGTDDSPIPITGYYIPCTHPKMSGLLRLGGESLVPPSANSSGSRNSCPVPGTLINTNNMRGLQNLDVEYLLREEAKKILHDIMYGKIEEDPSLLLRFLVISFADLKNWKIYYSVAFPSLVFKSEMTLLSLHSASLVLSQEEAKSLSKSLKEWRSSNETAALPFFFVDISSDSSIAIRQLKDWKDCQDNGQKFLFGFYDHGCHQDPGWALRNYIAFLSLRLKIEKIQFLCYREKRSELDLEKSLVGEASFPPPHGWDDSDYVPEAIGWEGEKPGDGRKEKKLKEINLESMSPERRDEEQQLMHLKLMGWRHFPVDLEKLSGIRCLLLGAGTLGCEVSRLLMTWGVRKLTVVDGGHVSMPDVLKQSLYVDDDCGVPRATAIVPHLKERCPAVDVEAIQMEIPAPGNPVSPSVLDDCERLQTLVASSDVVFLLTDTWESRWFPTLLCANENKMAITAALGHDSYLVMRHGAGPGTRRGNMDDVIAQIQNLSTEDALGRQRLGCCFCNDTTSLFNSVSDETVALPGLTSIVSGKAVELFARMLHHPEGIHAPGDMAGMDTEHQLGLLPHQMRGSLPRCDLSTVISNSSNNCTACSNIVLSEYRREGLNFVMQAINQPTYLKDLTGISNLIKSDTCLKLPASFPVNSGKLSSARCLLLGAGTLGCDVARILMDYGVRNLTIVDSGCVVVSNLARQSLYTTKDRKTPKATAILEHLKERCSSVEVEGIQMEIPMPGHPVSSKEAAGVLKACERLQELVAAHDAVFLLTDTRESRWLPTLLCANENKIAITAALGYDSYLAMRHGAGPGINSEGSDMVAAMNKLSAEDVLGRQRLGCYFCNDVIAPVDSVSNRTLDQQCTVTRPGLASIASGHAADLFTRLLNHPDGIHAPGDIAGTSSERPSGLLPHQMRGSLSQYSLLTLMGYSSSSCIACSNAVLREYRSRGLDFVMQVINEPTYLEDLTGLTELMKSADYSRVEWVDEVDDEDFADM

SEQ ID NO: 39 - DNA, 3066 bp, Zea mays
  CDS (1)..(3066): GRMZM2G005304
atggcgggtgccggtgcgatgggcttcccacgggagctgatggttcagcgtatacagagcttagtggaggaggggttctgggacgcgctccgccgccacaagctcgacgtcctcggcaccgacgactcccccatcccaatcaccggttattatactccgcgccagcgtcgcccgatggcaagcttcttcaacctacggtccggatcattggtgccaccatctcacagttctgttggcgacaggaacaattgcccagttccagggaccctcataaacactaataacatgaggggatttgaaaacctagatagagaacaacttctgaaagcagaagcaaagaagatcttgcatgacattgtgtctggtaaagtagaagaggatccttctgttctacttagattccttgtgacatcatttgctgatttgaagaattggaaggtctattacaatgttgcattcccctcattggttttcaactcaagaatcaccttgctcagcctgcaacctgcctcaaaagtgctcaccaaagaagaggcagcatccatgtatacatcattgcaaaagtggcgtacttctagtgaaacaacagttattccattctttctggttagtatatcctcagattcctctgcctctataagacaacttaaggaatggaaagcttgccaggggaactatcaaaagctgctatttggattttatgaccatggatgccgctcagattgccctggctgggttcttagaaactatgttgcctttctgagcatccgatggaagattgagaaagcccagattttctgctaccgtgaatataaagggaaccctgatctagaacagtccctcattggtgaagcatcatttccatcaccttatggttgggataaccctgaatttctacctgatgctattggatgggaaggagtaaagccagggaagggaacaaaagaaatgaaaccaaaagaaatagatcttcaatcaatgaatccagcaagtcaggatgaagaaaaacaattgatgcacttaaagcttatggggtggcgccactttcctgtgaatatagacaggttatcacatgttcgagttcttctcttgggtgctggaactcttggatgcgaagttgctcgccttcttatgacctggggtgtacggaaactaacggttgttgatagtggtagtgttgttatgtctgaccttgccaagcaatcactctacatagataaggattgtggagttccaagagtatctgcaatagctccacatctgaaagaaagatgtcctgcagtggaggttgaaagcatccaactggaaataccgatgcctgggcatcctgtttcttccagcaaaatggcaagtgtgcttgatgactgcaagcatctgcaaaccttagtggctgccaatgatgcaatcttcttgttaactgacacatgggagagcaggtggcttccaactcttctctgttcaagtgaaaacaagattgctattagtgcattattaggacatgacagttaccttgtcatgcgacatggtgctggaccaggaacaagtgggggtacagatgaagtgactactcagaaattgtccacagaaaatgctcttggtcatcaaagattgggctgttgtttctgcaatgataccgcttcccttattaatccaatttcttatgaaacactacctggacttacctcagttgcatctggcaaagcagtggagctctttgctaggatgttacatcatcctgacgagatacgggccccaggcgatactgctggtacagaaacagaacatcaacttggtctattgccacatcagctacgaggatcactccccaagtgtgatttatttgtggagctcaacaattcctcagtcaactgtactgcatgttctgttgctgtattgtcggagtatagaagaagaggattggattttgtcatgcaggctatcaactatccagcatatctgaaagattttacaggcatttctgattcgaaaagaccagatacttgtcctaaaataccgcccaacatctctgtaaattcagacaagatttctgatgtccgatgtctactattgggtgctggaactcttggatgtgatgtcgctcgcattcttatggactgtggtgtacggaagataacagtagttgatagtggtcatgtagttgtgtcaaatttagcgagacaatcactctatacttctgatgaccgtggtgctccaaaagcaactgcaatacttaggcatctagtggagagatgtccttcagtggatgcacaaggcattaaaatggaaatacctatgcctgggcatcctgtgtctcctggtgaagcagcgggtgtgctccaagattgcgagcgccttaaggaattggtgttgtcccacgatgctatcttcttgttgaccgacacaagagaaagcaggtggcttccaactctcctatgcactaacgaaaacaagattgctatcactgcagcattagggtatgatagttaccttgtcatgcgacatggggctggccctggcataagctgtgaagcttccagtgtggtcactgccatagacaagatgtccacggaggatgcccttgggcgtcaaaggctggggtgttatttctgcaatgacgtcattgctcctgtcgactcagtttccaatcggacactggatcaacaatgtacagtgacacgaccggggttggcctctattgcatcaggacgcgctgcagacctcttcagaagaatgttacaccatcctgatgggatacatgctccaggagagattgctggtacaggcagtggtcatgagcacggccttttacctcaccagatgcgtggatcactatcgcagtacaacatattaaccctgttgggctattcctcgagtaattgcacggcatgctcccatgcggtattgtatcaatatcggaggagagggatggatttcgtgatgcaagtaatcaatgaaccaacctatttggaagacctgaccggcctcacggagttgatgaaatccgcgtcttgctcacaggtcgagtgggtagatgaaattgatgaagacgatttcgtcgaggtctga

SEQ ID NO: 40 - protein, 1021 aa, Zea mays
MAGAGAMGFPRELMVQRIQSLVEEGFWDALRRHKLDVLGTDDSPIPITGYYTPRQRRPMASFFNLRSGSLVPPSHSSVGDRNNCPVPGTLINTNNMRGFENLDREQLLKAEAKKILHDIVSGKVEEDPSVLLRFLVTSFADLKNWKVYYNVAFPSLVFNSRITLLSLQPASKVLTKEEAASMYTSLQKWRTSSETTVIPFFLVSISSDSSASIRQLKEWKACQGNYQKLLFGFYDHGCRSDCPGWVLRNYVAFLSIRWKIEKAQIFCYREYKGNPDLEQSLIGEASFPSPYGWDNPEFLPDAIGWEGVKPGKGTKEMKPKEIDLQSMNPASQDEEKQLMHLKLMGWRHFPVNIDRLSHVRVLLLGAGTLGCEVARLLMTWGVRKLTVVDSGSVVMSDLAKQSLYIDKDCGVPRVSAIAPHLKERCPAVEVESIQLEIPMPGHPVSSSKMASVLDDCKHLQTLVAANDAIFLLTDTWESRWLPTLLCSSENKIAISALLGHDSYLVMRHGAGPGTSGGTDEVTTQKLSTENALGHQRLGCCFCNDTASLINPISYETLPGLTSVASGKAVELFARMLHHPDEIRAPGDTAGTETEHQLGLLPHQLRGSLPKCDLFVELNNSSVNCTACSVAVLSEYRRRGLDFVMQAINYPAYLKDFTGISDSKRPDTCPKIPPNISVNSDKISDVRCLLLGAGTLGCDVARILMDCGVRKITVVDSGHVVVSNLARQSLYTSDDRGAPKATAILRHLVERCPSVDAQGIKMEIPMPGHPVSPGEAAGVLQDCERLKELVLSHDAIFLLTDTRESRWLPTLLCTNENKIAITAALGYDSYLVMRHGAGPGISCEASSVVTAIDKMSTEDALGRQRLGCYFCNDVIAPVDSVSNRTLDQQCTVTRPGLASIASGRAADLFRRMLHHPDGIHAPGEIAGTGSGHEHGLLPHQMRGSLSQYNILTLLGYSSSNCTACSHAVLYQYRRRGMDFVMQVINEPTYLEDLTGLTELMKSASCSQVEWVDEIDEDDFVEV

SEQ ID NO: 41 - DNA, 51 bp, Artificial Sequence
  attB1-ATG5UTR-Fw Primer
ggggacaagtttgtacaaaaaagcaggctatacagaaacagcgtcgttttg

SEQ ID NO: 42 - DNA, 53 bp, Artificial Sequence
  attB2-ATG5-Rev Primer
ggggaccactttgtacaagaaagctgggttcacctttgaggagctttcacaag

SEQ ID NO: 43 - DNA, 25 bp, Artificial Sequence
  AtATG5 qPCR Fw2 Primer
atacactttagaggatatccttgca

SEQ ID NO: 44 - DNA, 23 bp, Artificial Sequence
  AtATG5  qPCR Re2 Primer
accgttcatgacagaggtccata

SEQ ID NO: 45 - DNA, 23 bp, Artificial Sequence
  AtATG7  qPCR Fw2 Primer
tctaatccagtcaggcaatctct

SEQ ID NO: 46 - DNA, 24 bp, Artificial Sequence
  AtATG7 qPCR Re2 Primer
gattcaatcaactcgctaaggcgt

SEQ ID NO: 47 - DNA, 19 bp, Artificial Sequence
  NBR1 qPCR Fw Primer
gaggacccagaccggaagg

SEQ ID NO: 48 - DNA, 20 bp, Artificial Sequence
  NBR1 qPCR Re Primer
gacaaacacgacgaggatgc

SEQ ID NO: 49 - DNA, 20 bp, Artificial Sequence
  PP2A qPCR Fw Primer
taacgtggccaaaatgatgc

SEQ ID NO: 50 - DNA, 20 bp, Artificial Sequence
  PP2A qPCR Re Primer
gttctccacaaccgcttggt

SEQ ID NO: 51 - DNA, 21 bp, Artificial Sequence
  HEL qPCR Fw Primer
ccattctactttttggcggct

SEQ ID NO: 52 - DNA, 26 bp, Artificial Sequence
  HEL qPCR Re Primer
tcaatggtaactgatccactctgatg

SEQ ID NO: 53 - DNA, 52 bp, Artificial Sequence
  FWatg7 Primer
ggggacaagtttgtacaaaaaagcaggcttgatggctgagaaagaaactcca

SEQ ID NO: 54 - DNA, 52 bp, Artificial Sequence
  RVatg7 Primer
ggggaccactttgtacaagaaagctgggtattaaagatctacagctacatcg

SEQ ID NO: 55 - DNA, 17 bp, Artificial Sequence
  UBQ 5 FW Primer
gacgcttcatctcgtcc

SEQ ID NO: 56 - DNA, 16 bp, Artificial Sequence
  UBQ 5 RV Primer
ccacaggttgcgttag

SEQ ID NO: 57 - DNA, 20 bp, Artificial Sequence
  PR2 FW Primer
aggagcttagcctcaccacc

SEQ ID NO: 58 - DNA, 22 bp, Artificial Sequence
  PR2 RV Primer
gaggatgagctcgatgtcagag

SEQ ID NO: 59 - DNA, 20 bp, Artificial Sequence
  Cutinase FW Primer
atcactgccggtggttactc

SEQ ID NO: 60 - DNA, 20 bp, Artificial Sequence
  Cutinase RV Primer
cgacacccttgatttggtct

SEQ ID NO: 61 - DNA, 253 bp, Unknown organism
  terminator (1)..(253): From Nopaline synthase
gatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatc

SEQ ID NO: 62 - DNA, 2070 bp, Arabidopsis thaliana
  Gene (1)..(2070): protein phosphatase 2A subunit A3 transcript
cggtctcatttctcgttcttctgattgatagatcgctcggaacttggaaagcagcgtaatcggtagggagtgatttgagttttggtgaggatgtctatggttgatgagcctttatacccgattgctgtgcttatcgacgagctaaaaaacgatgatattcagcgtagattgaactctattaaacggctttctatcattgctcgtgctcttggagaggagaggacaagaaaagagttgattccatttcttagtgagaacaatgacgatgacgatgaggtgcttttggctatggcggaagagttggggggttttattctgtatgtaggaggggttgagtatgcatatgttctgcttccacctttggagactctatccactgttgaggaaacttgcgtgagggagaaagctgtggattcactttgtagaattggtgctcagatgagggagagtgacttggttgagcatttcactcctctggctaagcgactttcagctggtgaatggttcacagccagagtatcagcatgtgggattttccatattgcatacccaagtgccccagatgtgctaaagacggagctaagatcaatatatggtcagctttgtcaagatgacatgccaatggtgcgcagagctgcagcaactaatttggggaagtttgctgctacaattgaatcagctcatttgaagacagacattatgtccatgtttgaggatcttacgcaagatgatcaagattcggttagattattggctgttgagggttgtgctgctcttgggaaattgttggagccccaggactgtgttgcacacattcttcctgtgattgtcaatttctcgcaggataagtcctggcgtgtgcgttatatggttgcaaatcagctctatgaactttgtgaagctgtaggaccggagccaactaggacggatctggtgcctgcatatgctcgtctactttgtgataatgaggcagaagttcggatagcagctgctggaaaagttaccaagttttgtcgcattttaaaccctgaactcgctatccagcacattcttccctgtgtaaaggaattatcatcagactcttctcagcacgtcagatctgcattggcatcagttataatgggaatggctccagtcttgggtaaggatgcaacaattgagcatcttcttccaatctttctttctctattgaaagacgaatttcctgatgtacgcttaaacattatcagcaaacttgaccaagtgaaccaggttattgggattgatctactatcacaatcgttactgccagccattgtagaacttgctgaagacaggcactggagagtacgtctggctataatcgagtatattcccttgttggccagtcaattaggtgtaggcttctttgatgagaagcttggtgctctttgcatgcaatggttacaagacaaggttcactcaatccgtgaagctgctgcaaacaatctgaagcgtcttgctgaagagtttggtcctgaatgggcaatgcagcatatagttcctcaggttctagagatgattaacaacccacactatctatatcggatgacgattcttcgtgcagtatcgcttctcgctccagtaatgggatccgagatcacatgttccaaactcttacctgcggtaataactgcatctaaagacagagttccaaacatcaaatttaacgtggccaaaatgatgcaatctctcattccgatagtcgaccaagcggttgtggagaacatgatacggccatgcttggtggagctaagtgaagacccagatgttgatgttcggtatttcgcaaatcaagctctccaatctattgacaatgtgatgatgtctagctaaaaaaaggtaaagaagacagcaacgaattgtgtttggttctcatgagattttgtaaacgatactttgtcgtgtgttgtcttttactttacacgtacgtgaccattgtttctctctgctactaatgttaatgttggcttcatgttttctgtgatttgttcgttgggcgtatttgctttttggtgcttaattttgtttagtccaaataatttacttatc

SEQ ID NO: 63 - DNA, 4254 bp, Arabidopsis thaliana
  Gene (1)..(4254): RNA helicase family protein
atggctccgacgaagaaacctcagaagaacaaacaaagcaaaaacgaaatagcttcttctttaatacctaattcaggccataagaaaccatctaaagccccaaagcttttaatttcgccggaaaacgaagaccgtttacgacggctcttgctcaactttcgccggacgccttctccggtcacggcgacgctatctgttactcagaagagaaagaaactcaataatctctacgagaatctctcttgcgaagggtttttagataatcagattgaactcgttctctcctctcttagggatggagcaacgcttgagactgctcttgattggctttgcttgaacttgccgagccatgaattgccagtgaacttttctaacggggcttctcgttttccttccacaggacgttctgtggcggtgatatcgaaatcaaagaaagattggaatgtgtctgctgaatcatcagtccaagaggttaaggaggtgcctgaatctgaagttttagttcgagtgaagagcaagcgagatgaagaagaagaagatagtcttagttcttgccaaccatctcaagctgattggatccatcagtatatgaaaagactggaagaggaggaattggaatcttccgacgatgagagagataaggtatctggtccaaggtctttcgaagtgattgccaaagaatattgtgtagaaagatataatgctataaaagcaaagaggaagggtgataaaagtggacagagccaagcaggcctagccatttgcaagctcaaggaagagatgaatgctcttgggccgtcagaagcaatattggaatctgagtttcagcgtgatcgtcaagattgtgaaggcgctagagaaaaagaagtagcctcctctgtgcttgatgacgttcatgagtcagtaaatgctgatgctttcttttttcagctttttgatgacttgaccttggatacaaatcctgttgggagctgtaaatctgaggaacaagaatcagttgtctcgaatgatagtttagatgacttggaccttggcgatttgttcgacgaagatgttccgccttatgtaccttctccccatgaactcctggaattacagaagaaggaaattatgagagaactatgcaatgagaaacatcttacgaaactaaacggtatctggaagaagggtgaagcgcaaaagatcccgaaggcattgctccatcagttgtgtcaaaggtcagggtggatagcaccaaagttcaataaagtgactggggaaggaagaaacttctcatatactacaagtgttatgcgtaaatccagtggatttggtaaaagcagacaagccgggggtctggtaactattcaacttcctcatcaagttgaggattttgaatccatccaggatgcacaaaacagagttgcggcatttgctctccataagctattctctgatttgcctgttcactttgcaattactgagccttatgcctctctagttttgatctggaaacaagaggaatcattaggtataacaagcagagaagagcaaagaagagaaaaatttgttgaaagcttgcttgaggcagacaatttcagcttgactactacttcgagaggcatacacagtgcacttccaatggtagactcctgtgtcaaagagaatgatgacttggatgttgtcaaatctaaccacagagccagaagaaactcttccatggcagctgaatgttcttctctcaaacaaaaacaagaaaacaagaagaagatgcagaagtacaaggacatgttgaaaactagagctgctcttcctatatcagaagtgaaaaaggacatactgcagaagctgaaagaaaaagatgtcttggtggtatgcggagaaactggttccggaaagactacacaggttcctcaatttatactggatgatatgatcgattcagggcatggtggatactgtaatattatatgcacacaacctcgggctatcactgttgctcaaagagttgctgatgagcgctgtgaacctcctccaggcttcgacaactctgtggttgcttatcaagttcgtcatcaaaatgcaaggagtgataagacaaggctgctgttttgtactactggaatcctactgagaaaacttgtgggggatacgaccttgaaagatgttacacatattatagtcgatgaagtgcatgagcggtctcttatgggtgattttcttctcattattttgaagagcctgattgagaagcaatcctgggataacgcattacctaaactaaaagttatccttatgtccgctactgttgatgcacatcagttctcaagatattttggtcaatgtcctataattactgctcaaggacgaactcacccggttaccacttactttctggaggacatttatgagaggactaagtaccttctggcttcagattcacctgctgctctaagttctgatacatccattacagacaagcttggttctgtaaacgtccctagggggaaaaagaaccttatgttggctggctggggagacagttatttggtttcagaggacagtcttaacacgtcttacgattccattaaatatattgcaagtgctgttgttgactatgatcttcttgaagagttgatatgtcacattgatgacacttgtgaggaaggagccattcttgtgtttttgcctggaatgtcagaaatcaacatgctactcaataggttagctgcttcctatcgttttcgtggagcttctggagattggcttcttcctctacattcttctattgcatctacagaacagaaaaaggtgtttctgcgcccccctaaaggcatacggaaggttatcatagctacaaatattgccgagaccagtataacaattgaggatgtggtttatgtgattgatagtggaaaacacaaggaaaatcgctataacccacataagaaattgtcaagcatggtggaagattgggtatccaaagcaaatgcaagacaaagaatgggaagagctggacgtgtcaaacctggacattgcttttcattatacactcgccataggttcgagaagctcatgcgtccctatcaggttcctgagatgctgcgggtgcctttagtagagctgtgtttacatatcaaattacttggcttgggtcaaattaagccgtttctgtccaaggctttggaacctccgagtgaaagtgctataaactctgcaattttattgttgcacaaggtcggcgctcttgaaggggacgaagagttgacaccactaggacatcatttagcaaaacttcctgttgatttgttaattggaaagatgctgttatatggtggcatttttggttgcttatcacctattctctcgattgctgctttcttgagctgctgcaaatcgccgtttgtatatgccaaagatgagcaaaatgtggaccgggtgaaacttgctcttttgtctgataagctggagagttcgagtaatttaaacaacaatgatagacaatctgatcacctccttatggtggttgcatatgaaaaatgggtgaggatactgcatgagcaaggatttaaagctgcagagagtttctgtgagtcaaagtttttgaatagctcagtgatgcggatgatgagagagaggagagtagagtttggcatgttgcttgccgacatcgggctcatcaatcttcctaaaggcaaggggagaaggaaggagaattttgatgtttggttttctgacaaaactcaacctttcaatatgtattcacaagaacctgaagttgttaaggctatattatgcgcgggactatgtcctaatattgcagagggattagttaaccgtttaacaaagccagcggaagaaacacagcgttatgcagtatggcatgatggcaaaagagaagtccatatccatcgaaactctattaacaaaaactgcaaagcgtttcaataccctttcattgtgttccttgaaaagctcgaaacaaagaaagtggtatatctgcaagatacaactgttgtttctccgttctccattctactttttggcggctcaatcaatgttcatcatcagagtggatcagttaccattgatgggtggttaaagctaacagcaccagcacaaactgccgttttgttcaaagagctccggttaacccttcattccattctcaaagatctcatccgaaaacccgagaaatcaggtattgtccacaacgaagtcgtcaaatctatggtccatcttcttatagaagaaggcaaaccgcaacacacatga

SEQ ID NO: 64 - DNA, 2956 bp, Arabidopsis thaliana
  5′UTR (1)..(79)
  exon (80)..(184)
  Intron (185)..(438)
  exon (439)..(563)
  Intron (564)..(655)
  exon (656)..(722)
  Intron (723)..(972)
  exon (973)..(1062)
  Intron (1063)..(1466)
  exon (1467)..(1554)
  Intron (1555)..(1775)
  exon (1776)..(1919)
  Intron (1920)..(2204)
  exon (2205)..(2336)
  Intron (2337)..(2478)
  exon (2479)..(2741)
  3′UTR (2942)..(2956)
atacagaaacagcgtcgttttgaaagctgagttttttttgtgaagcgagcgagcgtgagaaggtgtgacggggaagagaatggcgaaggaagcggtcaagtatgtatgggaaggagcaattcctctgcagattcatctccacaaatccgacgtcgcttctcaccctgctcctcctcctgctcttgtaaatcacctttcttctccaagctttttttctcatgtctggttttcaaatcggtggtgcaaatctgtggtctttggtgttggatcatgatcgatttcgggttttaggggtttcgtttccatttcgtattgaatcatacgatttttgaccatcaaattgattgaaacgaggaagaatttagacgaaatttgtgtaactgggtcgagataagatcatacttctgaatttgctatttgtttggcacgcattatcaggtgttagcaccaagaataggatatttgcctctgttgattcctcttataaagccttatttcaaggattcacttcctcctggtgaagattcaatttggtttgattacaaaggatttcctctaaaatggtacgccattgtttcatctcttagtataatgggagtaagtgtttgttttttgtgatatgttatgtgagagtgtgaatcttctttggttgcaggtatataccaacaggtgttcttttcgatctcctttgtgcagaacccgaaagaccatggaatctcacggtaatttttcagattgaactttgtattaggatcttaagtaagcctgagggaaaaatattcttgtaatctaagcttaagtggacaatgttaatgatccactatgtcattctcttattcttgtaagctgacactcgtaagtatgttattaacaagatctttcaccagtgtccctcttgactagctaattgcacaaagcttacctccttatatctttgcatttcttgagcttttctgaaaattttgcttgcagatacactttagaggatatccttgcaacatactgataccatgtgaaggagaagattctgtaaaatggaactttgttaattctttgaaagaggttagtgaaaatttcttcaaagtttatgactttcttatcattttaagtgaaagtctatgttcaaattattttcgaatagactataaatactgttctattggatttcacatcgtagaacagtgaaatatttgttacctttgggagtgtttctcttttgttataagagtggttttcttcaacatattgatgatgtattgtctatataggtgtagctgcgtatatccgcaaatagtattgttagtaatttgttagttttgctgcaattggtttgccattatctaacgtaatacataggcttctcttgtgttatatttttacaatgctccatgccccaagtaaagaaaaaggataaatgataagacgttttgacatatgtgagatactgttatccgcttatcctgcaggcacaatatatcatcaatggaaattgcaagaatgttatgaacatgtctcagagtgatcaagaggatctatggacctctgtcatgaacggtacgagtggacgatgttaggcatatcaattactagcagtcacaaatcccttatcctttttgttacaccacttcttctggtcgtaactctagttgccactcgatgtaggtgtagtttttgcgttcagcattgccaatgtcactatttgcatcaaccatggtataaccgttggatatccatgactgatttagtaacgagtttgaaaactgatggtttggcaggtgatcttgatgcctatacaagattatcacccaagcttaaaatgggaacagtcgaagatgagttttcaaggaaaacaagtttgtcatctccacaatctcaacaagttgtgcctgagacggaggtggctggacaagttaagacaggtttgccaactatatactcagctgagacagttacaccttgtcaaattttcccatatttggaaaagtagtatcacaagatgggactggtatatttttatgcagaggacctagtaaaaacactgcttgtaagtagtagttttcccttttctgataaatcctataaacagcaacaacttaacaggaaaagaataatgacttttcacaaattgttgcatctattgcagtgatggcaaacatgatacaaagtactagcgtcctgatttaatgtaaaatcctttttttcagcaagaattcctgttcggttgtatgttcgaagtctaaataaagatttcgagaatcttgaagatgtaccggagatcgatacctgggatgacatctcgtaccttaatcgccctgttgagttcctcaaagaagaaggtaagaatttcatcttaatgatccacattgttgttattaccttgttcattgaaaagaacccaaaggtgttatgtagagtgaatccaactgttgttgagtctctctttgtgtgagataactgatttcttccatcatctaacagggaaatgctttacgttacgtgacgccattaaaagtctcctccctgagtttatgggagacagagcgcaaacgagtggtgaagaaagaagcatagatgatacagaagaagcagatgggtcgagggagatgggtgaaatcaaattggtaaggatacaagggatagaaatgaagctagagataccgttttcgtgggtggtaaataacttgatgaacccagaattctatctccatatctctgtccttgtgaaagctcctcaaaggtgaagtgtaaggttctctgcagttacaatccatctgtgaattgaatcaaattgctttctcgttccatcttacaaatccgaaagaatcaatgatttgttgtatacagctacttcttctatttctgatagaagcaagaacacagaatacagaaaagaaagtagaaatattttgattgactcgttagctcttttacatttgttacttgacggtttcctctc

SEQ ID NO: 65 - DNA, 3339 bp, Arabidopsis thaliana
  5′UTR (1)..(82)
  exon (83)..(221)
  Intron (222)..(501)
  exon (502)..(713)
  Intron (714)..(797)
  exon (798)..(1044)
  Intron (1045)..(1134)
  exon (1135)..(1229)
  Intron (1230)..(1308)
  exon (1309)..(1601)
  Intron (1602)..(1684)
  exon (1685)..(1829)
  Intron (1830)..(1915)
  exon (1916)..(2263)
  Intron (2264)..(2351)
  exon (2352)..(2513)
  Intron (2514)..(2609)
  exon (2610)..(2725)
  Intron (2726)..(2802)
  exon (2803)..(2965)
  Intron (2966)..(3041)
  exon (3042)..(3215)
  3′UTR (3216)..(3339)
cgtcatcgtcttcttctttctcacattcattgactctcctccataataaacattgcaatctctcagaaagataagatcagccatggctgagaaagaaactccagcaatcatacttcaattcgctccattgaacagttccgtcgacgaaggtttctggcatagcttctcctctttgaaactcgacaaactcggaatcgatgattctccgatttcaatcaccggtaaacacacaatcatcgaattaaaaaatcatccaatgcatcgatttttatatattattcgatgagtctatgcattcgagctcttatgatccgtttaggttagagaagcaaaaaaaaaagttgaaaacttgaaaatcctctgttttctgggcaagttgttatgatccgtttaggttagagaagcaaaaaaaaagttgagaactttaaaagcctctgttttgtttagtagagattattcagatttagaatttatgtgcttttttggaactgaaattttcaggtttctatggaccatgtggacatccacaagtctcaaatcatttgactcttttatcagagtcattgcctttagatgaacaatcattgattgctagtactagtcatggaaataggaataagtgtcctgttcctgggattttgtataacaccaataccgttgaaagcttcaataaacttgacaaacaaagtctgctaaaagcagaagcaaataaggtaaatacaaaacttagctccgtgacttggtttttactggttcttgtctgcagaagttttaacaattttgttggtgaattttagatttgggaggatattcaatcaggaaaagctcttgaagacccttctgtgttaccaagattcctcgttatctcctttgcagaccttaagaaatggagttttcggtattggtttgcgttccctgcgtttgtacttgatcctcctgtaagtttgattgaattaaagccagcttcagagtattttagttctgaagaggctgaatcagtatccgctgcttgtaatgattggcgcgactcagatttaactactggtactcaacattcttgttctattttttactattaaaattcaaggagtggtagctgaaaaccttttgttacttctccgtgttttgctgtagatgttccattctttttggtttcggtttcttctgattcaaaagctagtatcagacatcttaaagacttggaagcgtgccaaggtgatcatcaaaaggtatgtgttaactaactcttccatatgcgttagatacagaaaaaacaaaattcacctttatgtgtctctcttcctgcagttgcttttcggtttctatgacccgtgtcaccttcctagtaatcctggctggccacttcgaaactacttggcactgattcgctcaagatggaacctcgagacagtttggttcttctgctacagagagagtcgtggtttcgctgacttgaacctttctcttgttggtcaagcctcaattacactttcatcaggtgaatcagctgagactgtacctaactcagtgggatgggagcttaacaaagggaaacgtgtaccccgatccattagccttgctaattccatggatccaacaaggtacatttttgctctgtgtcaatgatctactcaactaaatgggtaaaatctgattagacaaacggaatcacgttctttcgcaggttggctgtttctgctgtcgatttaaacttaaagttaatgagatggcgagcattaccgtctcttaacttaaatgtattgtcctctgtcaaatgccttcttcttggagctggtacattgggttgccaagttgctcgtacccttatggtacgtattgaagaaaatgcgagatttgacattattttacaaactcactgattaactaagaactcgtttatcttttattttcttagggttggggaatccgcaatatcacctttgttgactatggaaaagttgccatgtctaatccagtcaggcaatctctctataattttgaagattgtctaggtcgtggtgagtttaaagctgttgctgctgttaaaagccttaaacagatatttccagcaatggaaactagtggagttgttatggctataccgatgcccggacatccgatttctagccaagaagaagattctgttctcggtgattgtaaacgccttagcgagttgattgaatctcatgatgcggtgttcttgttaaccgatacaagagaaagcaggtggttaccgtctcttctttgtgctaatgccaataaggtaactaactctgtttctttatttcttcttactggttatataagtataacaatctgcttgatatgtcggtgtttgatgtgaatttcagatcgctataaacgcggctttaggttttgacagctacatggtaatgcgccatggtgctggccccacctctctgtctgatgatatgcagaatcttgacataaacaagacaaatacacagagacttggttgttacttctgcaacgacgtggttgcacctcaggatgtaattccctggtttcttcattttgaattgttttgtttcgtgttacctccagttcagtcctgattcgtcgtacccactcttttcttgtatttgcagtcaatgactgatcgaactctagaccaacaatgcactgttacacgcccgggtctagctcctattgcaggagctctcgccgttgaactcttagtcggagtcctacaacacccacttgggtaagcctttcgaaactcgaatacattgaaaatacaatcctcaggcttatcgtcttacatatgtttattacatacagtatcaatgcaaaaggcgacaattcgagcttgagtaatacaggaaacaatgatgattcgcctctcgggatattacctcatcagatacgaggctcagtttctcagttctcacagatcacgctgcttggtcaagcttccaacagctgtaccgcttgctctgaaaccgtaagtttccacatgacatcattcttagaaaactgcgctcgagaatctgatgatgtttataaacttgtattttcaggtgatatcggagtatagggagagaggaaacagtttcatacttgaagctattaaccatcctacatacctggaggatcttactggtttaaccgagcttaagaaagctgctaattcattcaacctcgactgggaagacgatgatactgatgatgacgatgtagctgtagatctttaaagacagatttatgtatcaaatgtccatagactatattcactaggtggcttcttttatgaatcttttgataagcatttttttaaactaaattaaagtacaaagaaatgaagaaatcaaattctta

SEQ ID NO: 66 - DNA, 210 bp, cauliflower mosaic virus
  promoter (1)..(210): gi_234167_gb_S51061.1 35S {promoter [cauliflower mosaic virus, Genomic, 210 nt]
tccactgacgtaagggatgacgcacaatcccactatccttcgcaagacccttcctctatataaggaagttcatttcatttggagaggacacgctgaaatcaccagtctctctctacaaatctatctctctctattttctccataataatgtgtgagtagttcccagataagggaattagggttcttatagggtttcgctcacgtgttgag